(19) United States
(12) Patent Application Publication
(10) Pub. No.: US 2026/0117242 A1
(43) Pub. Date: Apr. 30, 2026

(54) CELL LINES, VARIETIES, AND METHODS FOR IN VITRO COTTON FIBER PRODUCTION

(71) Applicant: Galy Co., Boston, MA (US)

(72) Inventors: Kari Perez, Amherst, MA (US); David Dodds, Boston, MA (US); Paula Maria Elbl, Boston, MA (US)

(21) Appl. No.: 18/272,488

(22) PCT Filed: Jan. 14, 2022
(86) PCT No.: PCT/US2022/012494

§ 371 Date: Jul. 14, 2023

Related U.S. Application Data
(60) Provisional application No. 63/137,952, filed on Jan. 15, 2021.

(51) Int. Cl. C12N 15/82; A01H 4/00

(52) U.S. Cl. CPC C12N 15/8262; A01H 4/005

(57) ABSTRACT

The present disclosure provides in vitro methods for producing cotton fiber, including methods using cotton cells that include and express one or more selected genes.

### REFERENCE TO RELATED APPLICATIONS

This application claims the benefit of and priority to U.S. Provisional Application No. 63/137,952, filed Jan. 15, 2021, the contents of which are hereby incorporated by reference in their entirety.

### FIELD

The present invention relates to in vitro methods of cotton fiber production using cotton cells that express selected genes.

### REFERENCE TO SEQUENCE LISTING

The material in the ASCII text file, named “GALY-68827US1_Sequence_Listing_ST25.txt”, created Nov. 3, 2023, file size 180,224 bytes, is hereby incorporated by reference.

### BACKGROUND

Cotton is the most widespread non-food crop in the world. However, cotton production is expensive both in terms of money and resources required for its successful cultivation. For example, cotton is a water-intensive crop, with an estimated 9,000-17,000 liters of water required for each kilogram of cotton fiber produced. This equates to enough drinking water to sustain 5,000 people for a day used in order to produce enough cotton to make two t-shirts. Similarly, cotton cultivation requires land, which must be otherwise diverted from other crop production, such as food production. It is estimated that for every acre of cotton grown, only about 500 kilograms of cotton fiber is produced. Cotton cultivation is also a net-emitter of greenhouse gasses, with approximately between 0.75 and 2.25 kilograms of carbon dioxide gas emitted per kilogram of cotton fiber produced. Moreover, because cotton is a plant, its cultivation can lead to failed crops, mistimed crops, and even excess production. Every year, billions of dollars are spent on logistics to overcome unexpected cotton harvest results.

In vitro production of plant cell compositions can overcome a number of limiting factors associated with in planta production of plant-derived products, thereby providing a reliable, energy-efficient, and eco-friendly alternative to traditional agriculture. For example, plant cell compositions produced in vitro can be continuously available, while crops grown in planta are often subject to a cyclic availability.

However, there are no currently known methods for the in vitro production of cotton fiber, especially at an industrial scale. The speed and scale of in vitro production of plant cell compositions currently remain limited by a number of engineering constraints, such as the difficulties of preparing a sufficient amount of cell inoculum of sufficient cellular homogeneity or the lack of streamlined protocols for an in vitro plant cell production cycle.

Moreover, there has been no identified genes or traits in cotton associated with amenability to in vitro cotton production.

### SUMMARY

The present invention provides methods and compositions for the in vitro production of cotton fiber using cotton cells that express selected genes of interest. These genes can make the cells particularly amenable to in vitro methods of cotton production. Cotton cells expressing the selected genes may thus show improved in vitro cotton fiber cell development compared to wildtype control cells. For example, the selected genes may improve in vitro fiber production by increasing the speed of cotton fiber cell development and/or increase the quantity cotton fiber cells. The selected genes can also lead to unexpectedly quick cell growth, fast cell multiplication/duplication, early cotton fiber/pre-fiber growth, and/or efficient bioreactor inoculation.

In certain aspects, the selected genes may be introduced into cotton cells as transgenes, and the modified cotton cells used in the presently disclosed in vitro methods of cotton fiber production. Thus, the selected genes of interest can be introduced into cells and the cells used to produce cotton fiber without the need to grow cotton plants. The methods and compositions of the invention can be scaled up, thereby allowing industrial scale production of cotton fiber.

By using the presently disclosed methods of in vitro cotton production, cotton fiber can be produced using approximately 77% less water and 80% less land than traditional in planta methods. Concurrently, the methods can produce cotton fiber and result in approximately 84% less carbon dioxide emissions when compared with traditional methods. Despite the lower resource costs, the methods of the invention produce cotton fiber much faster that in planta methods. Whereas cotton traditionally requires 5-6 months from planting to harvest, the in vitro methods of the present disclosure can lead to a cotton fiber harvest in approximately 45 days or less. Additionally, because the disclosed methods are in vitro as opposed to in planta, they can be more rigidly controlled. Therefore, the propensity for failed, mistimed, or excess crops can be reduced, if not completely, eliminated.

The present invention includes a method for producing cotton fiber that includes modifying cotton cells such that they comprise at least one differently expressed gene; culturing the modified cotton cells in vitro; and harvesting cotton fiber from the cultured cells. In certain aspects, the differently expressed gene affects growth of the modified cotton cells and/or cotton fiber development.

In certain aspects, the cotton fiber is harvested directly from the cultured cells. The cotton fiber may be harvested from the cells without growing a cotton plant.

In certain methods, the differently expressed gene is a gene selected for improvement and/or modulation of cotton fiber development or a developmental regulatory gene. The differently expressed gene may be a transgene. The transgene may be operably connected to a promoter. The promotor may be selected from a D113 promoter, a GbPDF1 promoter, a GhMYB109 promoter, a Stunt7 (S7) promoter, a floral binding protein 7 (FBP7) promoter, a TA29 promoter, and a CaMV 35S promoter, a cotton fiber-specific promoter, such as a GhSCFP promoter, a GaRDL1 promoter, an actin promoter, such as a cotton GhACT1 promoter, a BAN gene promoter, a petunia MADS box gene floral binding protein 7 (FBP7) promoter. In certain aspects, modifying the cotton cells may include introducing a transgene and promoter via transformation.

The differently expressed gene may be expressed at a higher level compared with a cotton cell prior to the modifying. In certain aspects, the differently expressed gene is a developmental regulatory gene. The differently expressed gene may decrease doubling time of the cells during the culturing. The developmental regulatory gene may be selected from Wuschel2 (Wus2); an Arabidopsis Wuschel gene (AtWUS); GhWus; and BABY BOOM (Bbm). a

The differently expressed gene may be a gene selected for improvement and/or modulation of cotton fiber development. A gene selected for improvement and/or modulation of cotton fiber development may improve/modulate cotton fiber development in cotton cells and/or cotton plants. The gene may be endogenous to one or more species or varietal of cotton plant, may be a gene from, or derived from, another species, or may be a synthetic gene. The gene may be, or derived from, a cotton fiber development gene. The gene may increase cotton fiber yield, increase cotton fiber length, and/or increase cotton fiber strength. The gene may be selected from homeodomain-leucine zipper transcription factor GhHOX3; putative homeodomain leucine zipper transcription factor GhHD-1; SQUASOMA promoter-binding protein-like transcription factor 5 (SPL5); GL1-like MYB protein encoding gene GaMYB2; a IAA biosynthetic gene iaaM; auxin efflux protein gene GhPIN3a; a cotton sucrose synthase gene (GhSus); cotton PROTODERMAL FACTOR1 (GbPDF1); cotton fiber specific RDR3 MYB gene GhMYB109; cotton LIM protein gene GhWlim5; basic helix-loop-helix protein encoding gene GhFP1; and a sucrose-synthesis related gene.

In certain aspects, the methods of the invention include methods in which prior to modifying the cotton cells such that they comprise at least one differently expressed gene, the cotton cells are obtained or are the progeny of cotton cells obtained from at least one cotton plant. The cotton cells may be obtained or are the progeny of cotton cells obtained from cotton plant or explant apical meristems, cotyledons, young leaves, hypocotyls, ovules, stems, mature leaves, flower, flower stalks, floral whorls, roots, bulbs, germinated seeds, somatic and zygotic embryo, and/or cambial meristematic cells (CMC).

In certain aspects, the modified cotton cells are not transgenic. The differently expressed gene may be endogenous to the modified cotton cells.

In certain methods of the disclosure, modifying comprises contacting the cotton cell with at least one mutagen, wherein the mutagen comprises an endonuclease, a chemical mutagen, and/or radiation.

In certain aspects, the methods of the present invention can be used to produce at least 1 kilogram of cotton fiber for every 4,000 liters of water used in the method. In some instances, the methods of the invention can produce at least 1 kilogram of cotton fiber for between every 2,000 and 4,000 of water used in the method.

### BRIEF DESCRIPTION OF THE DRAWINGS

FIG. 1 shows an exemplary method of the invention

FIG. 2 shows a flowchart of the concept of a commercial scale process for the cotton fiber in vitro production.

FIG. 3 shows a computer system that is programmed or otherwise configured to implement methods provided herein.

### DETAILED DESCRIPTION

The present invention provides methods and compositions for the in vitro production of cotton fiber using cotton cells that express selected genes of interest. Due to the ubiquity of cotton production around the world, there has long been interest in modifying cotton plants, through genetic alterations or selective breeding, in order to produce cotton plants with commercially-relevant characteristics. This may lead to cotton plants that include traits that result in better growth under certain agronomic conditions, pest and disease resistance, and increased yield. Cotton plants have also been modified to improve cotton fiber-related traits. For example, cotton plants have been developed with increased fiber length, strength, amenability to dye, and decreased fuzz fiber, immature fiber content, micronaire, fiber uniformity, and fiber maturity ratio. The present invention includes, for the first time, methods and compositions in which cotton cells that express selected genes, which lead to the aforementioned improvements, when the cells are used in methods of in vitro cotton fiber production. Moreover, due to the unique nature of the disclosed methods, the selected genes may provide improvements uniquely tailored to in vitro cotton fiber production.

The methods of the disclosure can be cell-based, and not require the growth of entire cotton plants for modifying cotton cells to express selected genes of interest and/or cultivation of cotton fiber. These methods allow quick and efficient cultivation of cotton fiber in a controlled environment. Cotton cells expressing the selected genes may show improved in vitro cotton fiber cell development compared to wildtype control cells. For example, the selected genes may improve in vitro fiber production by increasing the speed of cotton fiber cell development and/or increase the quantity cotton fiber cells. The selected genes can also lead to unexpectedly quick cell growth, fast cell multiplication/duplication, early cotton fiber/pre-fiber growth, and/or efficient bioreactor inoculation.

In certain aspects, the selected genes may be introduced into cotton cells as transgenes, and the modified cotton cells used in the presently disclosed in vitro methods of cotton fiber production. Thus, the selected genes of interest can be introduced into cells and the cells used to produce cotton fiber without the need to grow cotton plants. The methods and compositions of the invention can be scaled up, thereby allowing industrial scale production of cotton fiber.

While cells derived from most, if not all, cotton varietals can be used for these in vitro methods of cotton production, some varietals possess traits that make them particularly useful for the presently disclosed methods of in vitro cotton fiber production. When used in the methods of the invention, some of these varietals show, for example, unexpectedly quick cell growth, fast cell multiplication/duplication, early cotton fiber/pre-fiber growth, and/or efficient bioreactor inoculation. In certain aspects, genes and/or gene expression patterns leading to improved in vitro cotton fiber production can be determined. Once determined, these genes and/or gene expression patterns can be introduced into other cotton plants/cells, for example, as a transgene.

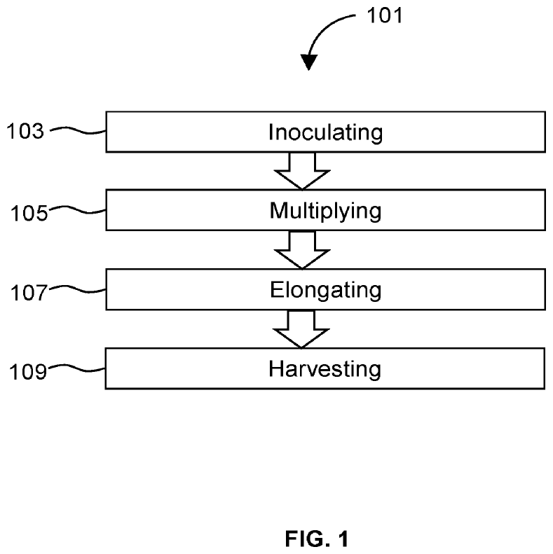

FIG. 1 provides an exemplary method 101 of the invention for the in vitro production of cotton fiber. As shown in FIG. 1, the method 101 begins with modifying or selecting cotton cells 103. The cotton cells are modified 103 to express, or selected 103 for expression, of one or more genes of interest. The cotton cells, or cells derived therefrom, can then be used to inoculate 105 a bioreactor. After inoculating 105, the method 101 requires multiplying 107 the cells in the bioreactor. The multiplied cells are then elongated 109 to produce cotton fibers. When the cotton fibers are sufficiently matured, the resulting cotton fiber is harvested 111.

The cotton cells may be modified to express, or selected for expression of, at least one selected gene.

The selected gene may include a gene selected for improvement and/or modulation of cotton fiber development in vitro. These genes may improve/modulate cotton fiber development in cotton cells and/or cotton plants. However, genes that do not modulate/improve cotton fiber development in plants may nevertheless modulate/improve cotton fiber development using the in vitro methods disclosed herein. The gene selected for improvement and/or modulation of cotton fiber development may be endogenous to one or more species or varietal of cotton plant, may be a gene from, or derived from, another species, or may be a synthetic gene. The gene may be, or derived from, a cotton fiber development gene. Expression or overexpression of genes such as GhHOX3, GhHD-1, SPL5, GaMYB2, iaaM, GhPIN3a, GhWlim5, and GhFP1 has been shown to have a positive impact on cotton fiber growth (Cai, C., et al., 2018; Liu, Z. et al., 2020; Shan, C. et al., 2014; Walford, S. et al., 2012; Wang, S. et al., 2004; Zhang, M., et al., 2011; Mei, G. et al., 2019; Iqbal, A. et al., 2020).

Overexpression of GhHOX3, a homeodomain-leucine zipper transcription factor has been shown to increase cotton fiber length, while silencing of GhHOX3 greatly reduced fiber length (Shan et al., 2014). Constitutive overexpression of GhHD-1, another putative homeodomain leucine zipper transcription factor, increased the number of initiating fibers initiating on the seed relative to the wild type control (Walford et al., 2012). When overexpressed in Arabidopsis, the cotton GaMYB2, which encodes a GL1-like MYB protein and is expressed early in developing fiber cells, was found to induce the production of seed trichomes in Arabidopsis, a plant that does not have seed trichomes (Wang et al., 2004). Transgenic expression of the IAA biosynthetic genes iaaM from Agrobacterium tumefaciens in cotton was found to increase the number of lint fibers, the lint percentage of the transgenic cotton, resulting in a >15% increase in lint yield compared to the wild type control (Zhang, et al., 2011). Cotton gene GhFP1 has been shown to positively regulate fiber elongation (Liu et al., 2020). The fiber length of the transgenic cotton overexpressing a cotton basic helix-loop-helix (bHLH) protein (GhFP1) was found to be significantly longer than wild-type (Liu et al., 2020). GhPIN3a, is an auxin efflux protein which is likely involved in auxin transport and regulation at fiber initiation, was found to localize in the outer integument in the developing ovule at 0 DPA (days post anthesis), demonstrating an important role for fiber initiation (Zeng, et al., 2019). Transcription factor, SQUAMOSA promoter-binding protein-like SPL5 was found to be highly expressed during fiber initiation (Cai, C. et al., 2018). Overexpression of GhPIN3a and SPL5 may increase the rate and quantity of fiber initiation. Expression of cotton gene GhWlim5 under the control of a fiber specific promoter resulted in plants with stronger cotton fiber (Iqbal, A. et al., 2020).

The selected gene may include a developmental regulator gene. In certain aspects, the cotton cells may be selected or modified to express at least one developmental regulator gene and/or at least one gene selected for improvement and/or modulation of cotton fiber development. Developmental regulator genes such as Wus2 and Bbm have been shown to have a positive impact in corn, increasing plant cell growth and transformation efficiency dramatically (Hoerster, G., 2020; Gordon-Kamm, B., 2019; Lowe, K., 2015; Lowe, K., 2018). In cotton, AtWUS was found to promote the formation of the embryogenic callus in cotton, promoting somatic embryogenesis and inducing organogenesis in cotton tissues cultured in vitro, yielding 3-4 times more embryogenic calli than the wild type control (Zheng, et al., 2014 and Bouchabké-Coussa et al., 2013). Cotton GhWUS gene was shown to have a positive impact on Arabidopsis transformation and regeneration in growth in vitro (Xiao, Y. et al., 2018). When transformed into cotton cells, such as ovule epidermal cells, these developmental growth regulators shorten the time and/or increase the number of cells that produce fiber when transformed either alone or in combination with one or more genes selected for improvement and/or modulation of cotton fiber development in vitro, such as GhHOX3, GhHD-1, SPL5, GaMYB2, iaaM, GhPIN3a, GhWlim5, and GhFP1. Tissue specific expression of developmental regulator genes Wus2 and Bbm has been shown to be essential for normal plant development in prior studies in corn (Hoerster, G., 2020; Gordon-Kamm, B., 2019; Lowe, K., 2015; Lowe, K., 2018). Therefore, tissue specific protomers were identified in cotton to ensure cotton fibers develop normally in vitro.

GhHOX3 means a gene encoding G. hirsutum Homeodomain-leucine zipper transcription (GhHOX3). GhHOX3 plays a role in fiber elongation. Increased expression of GhHOX3 can dramatically increase cotton fiber length, while silencing GhHOX3 reduces cotton fiber length by more than 80%. A complete coding sequence (CDS) of GhHOX3 and the amino acid sequence of GhHOX include those set forth in Table 1.

GhHD-1 means a gene encoding Putative homeodomain leucine zipper (HD-ZIP) transcription factor, GhHD-1. GhHD-1 is expressed early in trichomes and early fibers and plays a role in cotton fiber initiation. Constitutive overexpression of GhHD-1 has been shown to increase the number of fibers initiating on the seed, but affect leaf trichomes when transformed in cotton. Cotton plant lines with overexpression of GhHD-1 were also shown to provide statistically significant increases in the number of fiber initials relative to a wild type, of between 18.6-28.4% and 25-42%. Table 1 includes coding sequences for GhHD-1 homeologs and their amino acid sequences.

GhACT means a G. hirsutum actin gene. Actins in cotton plants are encoded by a multigene family that includes GhACT1, GhACT2, GhACT3, GhACT, GhACT5, GhACT6, GhACT7, GhACT8, GhACT9, GhACT10, GhACT11, GhACT12, GhACT13, GhACT14, and GhACT15. These actin genes have a high sequence homology of around 70%-97% identity and coding regions with 93%-99% identity. Of these actin genes, GhACT1, GhACT2, GhACT4, GhACT5, and GhACT11, were shown to be preferentially expressed in cotton fibers compared to other actin genes. GhACT1 plays a role in fiber initiation, rather than fiber elongation. It was shown that that silencing GhACT1 decreases cotton fiber length and elongation rates in plantae. However, silencing the gene caused no impact on flower development or vegetative growth. Further, due to the tissue and temporal preferential expression of GhACT genes in plantae, their promoter sequences can be used as promoters for the differentially expressed genes used in the methods and compositions of the invention. Coding sequences for GhACT1 and an amino acid sequences of GhACT1 include those set forth in Table 1.

GhWlim5 means a gene encoding G. hirsutum GhWlim5 protein. GhWlim5 domain 1 interacts with GhACTIN-1 proteins. Overexpression of GhWlim5 in transgenic cotton plants was shown to provide increased cotton fiber strength. Coding sequences of GhWlim5 include those as set forth in Table 1.

An SBP gene family gene means a gene encoding a SQUASOMA promoter binding-like protein, any of which may be used in the disclosed in vitro methods of cotton fiber production. An exemplary SBP gene family gene is SPL5. SPL5 means a gene encoding a G. hirsutum SQUAMOSA promoter binding-like transcription factor 5 (SPL5). Cotton may include paralogs and/or homeologs of SPL5, including GhSPL5a_A, GhSPL5a_D, GhSPL5b_A, GhSPL5b_D, GhSPL5e_A, GhSPL5c_D, GhSPL5d_A, and GhSPL5d_D. The SPL5 gene family is involved in the modulation of cotton boll density, number, and size and lint percentage in cotton plants. Genes of this family are involved in the control of early cotton flower development. Coding sequences for SPL5 gene family members include those set forth in Table 1.

MYB2 means a gene encoding a myb family transcription factor 2/fiber factor 1. GhMYB2 means such a gene of G. hirsutum. GaMYB2 means such a gene of G. arboretum. GbMYB2 means such a gene of G. barbadense. GaMYB21GhMYB2 GbMYB2 are Arabidopsis GLABRA1 (GL1) orthologues in cotton. GhMYB2 has been reported to have an amino acid motif for interaction with a bHLH protein. GaMYB2 expressed in A. thaliana under the control of GL1 promoter fully restored trichome formation in a gl1 mutant. GhMYB109 is another R2R3 MYB gene identified in upland cotton (G. hirsutum). Seeds of Arabidopsis are completely glabrous. It has been shown that observation of samples under binoculars did not find any trichomes on seed coat of wild-type plants. Seeds of transgenic 35S::GaMYB2 plants showed seed-trichome formation in both wild-type and gl1 backgrounds. In the wild-type background, ˜20% of plants showed seed-trichome phenotype, and among these plants ˜20% of the seeds produced epidermal trichomes. The trichomes were often unbranched or occasionally branched, and the number of trichomes on each seed was small. Other aspects of seed development appeared normal. In plants transformed with 35S::GL1, 35S::WER, and 35S::GaMYB109, seed trichomes were not observed. Exemplary coding sequences for MYB2 gene family members includes those set forth in Table 1.

WUSCHEL (WUS) genes encode a plant-specific homeodomain-containing transcriptional regulator, which plays important roles during embryogenesis, as well as in the formation of shoot and flower meristems. AtWus means a gene encoding a WUSCHEL (WUS) gene from A. thaliana, which is a homeobox transcription factor. Overexpression of AtWus in cotton has been shown to increase the differentiation potential of cotton calli by triggering the auxin transport and signaling pathways. Overexpression has been shown to yield 3-4 times more embryogenic calli than a control. Maize Wuschel2 gene (WUS2) has been shown to dramatically improve transformation efficiency in multiple crops. Coding sequences of AtWUS and WUS2 include those as set forth in Table 1.

GhWUS means a gene encoding a G. hirsutum WUSCHEL gene. GhWUS was shown to be an important regulator of somatic embryogenesis and shoot regeneration. GhWUS1a_At and GhWUS1b_At, two cotton homologues from G. hirsutum, induced embryo and shoot regeneration when transgenically expressed in Arabidopsis. Downregulation of GhWUS in cotton inhibited embryogenic calli formation Exemplary GhWUS coding sequences and amino acid sequences are set forth in Table 1.

The term iaaM means the biosynthetic gene iaaM from Agrobacterium tumefaciens. Expression of iaaM under the promoter of petunia MADS box gene Floral Binding protein 7 (FBP7) has been shown to increase levels of plant hormone indole-3-acetic acid (IAA) in the epidermis of cotton ovules at the fiber initiation stage. This, in turn, was shown to increase the number and percentage of lint fibers and increase fiber fitness. A nucleotide sequence of a plasmid that includes iaaM can be found in Table 1.

GhPIN means a gene encoding a G. hirsutum auxin efflux carrier. Ovule suppression of GhPIN genes in transgenic cotton plans inhibited fiber elongation and initiation. GhPIN-mediated auxin transport was shown to have an important role in fiber-specific auxin accumulation for fiber initiation. GhPIN3a among other GhPIN genes showed particular localization of the transcript in the outer integument. Coding sequences of GhPIN3a include those as set forth in Table 1.

GhFP1 means a gene encoding G. hirsutum basic helix-loop-helix protein FP1 (GhFP1). GhFP1 has been shown to promote cotton fiber elongation. Fiber length in transgenic cotton overexpressing GhFP1 was increased compared to that of wildtype. Suppression of GhFP1 expression hindered fiber elongation. Expression of the brassinosteroid (BR)-related genes was markedly upregulated in fibers of cotton plants overexpressing GhFP1 and downregulated in GhFP1-silenced fibers. BR content in the transgenic fibers was shown to be altered, relative to that in wildtype. GhFP1 protein was shown to directly bind to the promoters of GhDWF4 and GhCPD to activate expression of these BR-related genes. GhFP1 was shown as a positive regulator participates in controlling fiber elongation by activating BR biosynthesis and signaling. Coding sequences of GhFP1 include those as set forth in Table 1.

GhSuS means a sucrose synthase (Sus) gene from G. hirsutum. Sus genes have been identified as involved in controlling cotton fiber development. (Zeng et al. 2016). Several GhSus homologs and homeologs have been identified in G. hirsutum and other cotton species. Certain GhSus alleles have been associated with fiber-related traits such as fiber length, fiber micronaire, fiber strength, yield, and lint percentage. Id. Accordingly, Sus genes from other cotton species may be used as a differently expressed gene in certain aspects of the invention. Similarly, over expression of a potato Sus gene in cotton plants gave rise to improved leaf development, early seed development, and fiber elongation. Id. Accordingly, a differently expressed gene used in the present invention may be derived from a species other than cotton. An exemplary nucleic acid sequence for a GhSus gene, GhSus1, is provided in Table 1.

Bbm means a gene encoding a BABY BOOM (Bbm) protein, for example, maize (Zea maize) Bbm (Zm-Bbm). When Bbm was expressed, transgenic calli were recovered from over 40% of the starting explants, with most producing healthy, fertile plants. Maize Bbm and Wus2 genes stimulate transformation in sorghum (Sorghum bicolor) immature embryos, sugarcane (Saccharum officinarum) callus, and indica rice (Oryza sativa ssp indica) callus. Exemplary cDNA sequences of Zm-Bbm are set forth in Table 1.

In certain aspects, the selected gene includes any of the genes as set forth in Table 1, including mutants, allele, homeologs, paralogs, and homologs of any of the disclosed genes. In a certain aspect, the selected gene includes a nucleic acid sequence having at least 90%, 95%, 96%, 97%, 98%, or at least 99% sequence identity to anyone of SEQ ID NOS: 1, 3, 5-7, 9-10, 12-30, 33-36, and 38-45, or one or more fragments thereof. The selected gene may be a derivative of any one of SEQ ID NOS: 1, 3, 5-7, 9-10, 12-30, 33-36, and 38-45. Derivatives may include, one or more mutations, such as deletions, point mutations, restriction site alterations, nucleotide substitutions, additions and/or codon modifications. Derivatives may also include, for example, promoters, functional elements or modified functional elements.

Percent sequence identity refers to the percentage of identical nucleotides between two segments of a window of optimally aligned DNA. Tools and methods for alignment are well known in the art, for example, the local homology algorithm of Smith and Waterman, the homology alignment algorithm of Needleman and Wunsch, the search for similarity method of Pearson and Lipman. These algorithms may implemented as, or included in, computer programs, for example, GAP, BESTFIT, and FASTA. An identity fraction for aligned sequence segments refers to the number of identical components that are shared by aligned test and reference sequences divided by the number of components in the reference sequence segment. Percent sequence identity is shown herein as the identity fraction multiplied by 100. The comparison of one or more DNA sequences may of an entire or full-length sequence or a portion thereof, or to a longer DNA sequence.

TABLE 1

| | Corresponding |
|---|---|
| Description | SEQ ID NO |
| GhHOX3 complete CDS (GenBank: KJ595848.1) | SEQ ID NO: 1 |
| GhHOX3 amino acid sequence (GenBank: AIA58892.1) | SEQ ID NO: 2 |
| GhHD-1D complete CDS (GenBank: JN585952.1) | SEQ ID NO: 3 |
| GhHD-1D amino acid sequence (GenBank: AFO11042.1) | SEQ ID NO: 4 |
| GhHD-1D complete CDS, regions 1178 . . . 1398, 1500 . . . 1617, | SEQ ID NO: 5 |
| 1756 . . . 1944, 2033 . . . 2470, 2561 . . . 2662, 2775 . . . 2975, 3075 . . . 3249, | |
| 3364 . . . 3635, 3731 . . . 3826, 3915 . . . 4280 (GenBank: JN585952.1) | |
| GhHD-1A complete CDS (GenBank: JN585951.1) | SEQ ID NO: 6 |
| GhHD-1A complete CDS, regions 2006 . . . 2226, 2322 . . . 2439 | SEQ ID NO: 7 |
| 2578 . . . 2766, 2855 . . . 3292, 3384 . . . 3485, 3598 . . . 3798, 3898 . . . 4072, | |
| 4187 . . . 4458, 4554 . . . 4649, 4741 . . . 5106 (GenBank: JN585951.1) | |
| GhHD-1A amino acid sequence (GenBank: AFO11041.1) | SEQ ID NO: 8 |
| GhACT1 complete CDS (GenBank: AY305723.1) | SEQ ID NO: 9 |
| GhACT1 complete CDS region 15 . . . 1148 (GenBank: AY305723.1) | SEQ ID NO: 10 |
| GhACT1 amino acid sequence (GenBank: AAP73448.1) | SEQ ID NO: 11 |
| SPL5 CDS, region 41 . . . 589 (GenBank: KJ622313.1) | SEQ ID NO: 12 |
| GhSPL5a_A (Gene ID: Gh_A01G0447) CDS | SEQ ID NO: 13 |
| GhSPL5a_D (Gene ID: Gh_D01G0457) CDS | SEQ ID NO: 14 |
| GhSPL5b_A (Gene ID: Gh_A02G1694) CDS | SEQ ID NO: 15 |
| GhSPL5b_D (Gene ID: Gh_D03G0025) CDS | SEQ ID NO: 16 |
| GhSPL5c_A (Gene ID: Gh_A10G2217) CDS | SEQ ID NO: 17 |
| GhSPL5c_D (Gene ID: Gh_D10G0251) CDS | SEQ ID NO: 18 |
| GhSPL5d_A (Gene ID: Gh_A13G1251) CDS | SEQ ID NO: 19 |
| GhSPL5d_D (Gene ID: Gh_D13G1551) CDS | SEQ ID NO: 20 |
| GaMYB2 complete CDS (GenBank: AY626160.1) | SEQ ID NO: 21 |
| GaMYB2 complete CDS, bases 74 . . . 670 (GenBank: AY626160.1) | SEQ ID NO: 22 |
| GhMYB2 complete CDS (GenBank: AY999070.1) | SEQ ID NO: 23 |
| GhMYB2 complete CDS, regions 43 . . . 178, 257 . . . 717 | SEQ ID NO: 24 |
| (GenBank: AY999070.1) | |
| GbMYB2 complete CDS (GenBank: AY999069.1) | SEQ ID NO: 25 |
| GbMYB2 complete CDS, regions 43 . . . 178, 257 . . . 717 | SEQ ID NO: 26 |
| (GenBank: AY999069.1) | |
| GhMYB109 complete CDS (GenBank: KT751523.1) | SEQ ID NO: 27 |
| ATWUS complete CDS (GenBank: AJ012310.1) | SEQ ID NO: 28 |
| ATWUS complete CDS, regions 122 . . . 997 (GenBank: AJ012310.1) | SEQ ID NO: 29 |
| GhWUS gene Gh_A12G0552.1 complete CDS | SEQ ID NO: 30 |
| GhWUS gene Gh_A10G0884.1 complete CDS | SEQ ID NO: 31 |
| GhWUS1a_At amino acid sequence. | SEQ ID NO: 32 |
| GhWUS1b_At amino acid sequence. | SEQ ID NO: 33 |
| Zm-WUS2 nucleotide sequence (GenBank: EA275154.1) | SEQ ID NO: 34 |
| GhWlim5 complete CDS (GenBank: JX290321.1) | SEQ ID NO: 35 |
| GhWlim5 complete CDS, region 1 . . . 570 (GenBank: JX290321.1) | SEQ ID NO: 36 |
| Agrobacterium tumefaciens octopine-type Ti plasmid [iaaM plasmid] | SEQ ID NO: 37 |
| CDS (GenBank: AF242881.1) | |
| Amino acid sequence product of Agrobacterium tumefaciens octopine-type | SEQ ID NO: 38 |
| Ti plasmid (GenBank: AAF77123.1) | |
| GhPIN3a complete CDS (NCBI Reference Sequence: NM_001327195.1) | SEQ ID NO: 39 |
| GhFP1 CDS (Gene ID: Gh_A11G2808) | SEQ ID NO: 40 |
| GhFP1 CDS (Gene ID: Gh_A11G360500) | SEQ ID NO: 41 |
| GhFP1 CDS (Gene ID: Gh_A11G360500.1) | SEQ ID NO: 42 |
| GhFP1 CDS (GenBank: KR782251.1) | SEQ ID NO: 43 |
| GhFP1 CDS, region 143 . . . 1210 (GenBank: KR782251.1) | SEQ ID NO: 44 |
| Zm-Bbm cDNA sequence including 5′ untranslated region | SEQ ID NO: 45 |
| (GenBank: CS155772.1) | |
| Zm-Bbm cDNA sequence, region 128 . . . 2260 (GenBank: CS155772.1) | SEQ ID NO: 46 |
| G. hirsutum TM-1 A-subgenome sucrose synthase (GhSus1) | SEQ ID NO: 47 |
| gene complete CDS (GenBank: HQ143089) | |

In certain aspects, the selected gene may be an exogenous gene, which is a nucleic acid introduced into a cell. The exogenous gene may encode an expression product, i.e., an RNA and/or protein expressed in a cell. The transgene may include a heterologous gene from, or derived from, a different species (including a non-plant species), cotton varietal, and/or be partially or completely synthetic. The transgene may include a homologous gene from, or derived from, same species or varietal (homologous), relative to the cell being transformed. The transgene can include a homologous gene that occupies a different location in the genome of the cotton cell or is under different control, relative to the endogenous copy of the gene. An exogenous gene may be present in more than one copy in the cell. An exogenous gene may be maintained in a cell as an insertion into the genome (nuclear or plastid) or as an episomal molecule.

Expression of the selected gene in a cotton cell may lead to a corresponding expression product of interest. An expression product of interest may include, for example, at least one peptide, protein, and/or nucleic acid. The nucleic acid may be an RNA molecule. The RNA molecule may be capable of interaction with one or more genes, nucleic acids and/or proteins in the cotton cell. The RNA may inhibit one or more gene, nucleic acid, and/or protein in the cotton cell. Inhibitory RNAs may include, for example, miRNA, siRNA, mRNA, tRNA, sense RNA, antisense RNA, hairpin RNA, and/or ribozymes. In certain aspects, the expression product of interest is a protein. The protein may alter fiber development may lead to improved in vitro cotton fiber cell development compared to wildtype cells. For example, the protein may improve in vitro fiber production by increasing the speed of cotton fiber cell development and/or increase the quantity cotton fiber cells. The protein can also lead to unexpectedly quick cell growth, fast cell multiplication/duplication, early cotton fiber/pre-fiber growth, and/or efficient bioreactor inoculation. In certain aspects, the protein is a cotton developmental growth regulator and/or affect cotton fiber development.

In certain aspects, the cotton cells may be modified by the inclusion of a fusion or chimeric gene. Such genes may include a selected gene of interest operably linked to a promoter or promoter region. These genes may further include one or more functional elements and/or regulatory elements, which may alter the expression or activity of the chimeric and/or selected gene of interest or a linked promoter. A chimeric gene or fusion gene may also include a transcription termination sequence and/or a polyadenylation sequence. The chimeric or fusion gene may operably link a selected gene of interest with an unrelated nucleic acid sequence, such as a promoter, or another gene. The chimeric or fusion gene may be a gene that is not normally found in a cotton plant. Table 3 provides non-limiting examples of fusion/chimeric genes used in the methods and cells of the invention.

Similarly, in certain aspects, the cotton cells may be modified by the inclusion of a fusion gene.

In certain aspects, the cotton cells are modified by the inclusion of a transgene/chimeric gene that, by its inclusion, modulates the level of an expression product endogenous to the cotton cell. For example, the transgene/chimeric gene may encode a protein endogenous to the cotton, and expression of the gene increases the level of protein that would otherwise be produced in the cotton cell. Similarly, the transgene/chimeric gene may modulate the temporal and/or spatial expression and/or levels of an expression product.

In certain aspects, the transgene/chimeric gene is operably linked to a promoter or promoter region. The promoter region may by homologous to the selected gene of interest. In certain aspects, the promoter and selected gene are not normally associated in nature. The promoter may modulate the expression of the gene. Modulated expression may lead to an increase or decrease in the expression of the gene. Modulated expression may also or alternatively modulate the spatial and/or temporal expression pattern of the selected gene. The chimeric gene/transgene can be synthesized to include a promoter that assures the selected gene of interest is expressed at a certain level, under specific growth conditions, in certain tissues and tissue types, and/or under specified spatial and/or temporal constraints.

Exemplary promoters include cotton-specific promoters or promoter regions of endogenous cotton genes that have temporally and/or spatially regulated expression, including late embryogenesis-abundant gene D113 from cotton accumulates at high levels in mature seeds, leaves, embryos, and callus (Luo et al., 2008), GbPDF1, cotton PROTODERMAL FACTOR1 gene (GbPDF1) which is predominantly expressed in the epidermis of ovules and developing fibers during fiber initiation and early elongation (Deng et al., 2012), and GhMYB109, a cotton fiber specific R2R3 MYB gene (Pu et al., 2008). Similarly, transgenic reporter gene analysis has shown that a 2-kb GhMYB109 promoter was sufficient to confirm its fiber-specific expression via GUS staining. A GhSCFP promoter was shown to specifically activate transcription in seed coat and fiber associated genes.

In some cases, non-cotton specific constitutive protomers such as 35S promoter (such as CaMV 35S), an FBP7 (petunia floral binding protein 7) promoter, a tobacco TA29 promoter, would function best, in the in vitro methods of the disclosure. Exemplary 35S, FBP7, and TA29 promoter nucleic acid sequences are provided in Table 2. In an exemplary alternative, a cotton-specific constitutive promoter such as the GhUbi14pro promoter, which is a constitutive endogenous promoter from G. hirsutum from the cotton ubiquitin gene.

In some cases, rather than a constitutive promoter, an introduced gene/genes is under the control of an inducible such that the expression of the gene may be controlled, e.g., stopped and started. Inducible promoters may be endogenous or exogenous. In certain aspects, inducible promoters may include heat shock promoters. The heat-shock response is a mechanism for cellular protection in response to adverse environmental growth conditions, which in natural circumstances results in the production of various heat-shock proteins. Sensory biomolecules sense temperature changes and transduce intercellular signals to coordinate gene expression outputs. Sensory biomolecules, also known as thermosensors, include nucleic acids (DNA or RNA) and proteins. Once a stress signal is perceived, it is transduced to invoke specific molecular mechanisms controlling transcription of genes coding for heat-shock proteins. Exemplary inducible heat shock promoters useful in the present invention include, for example, AtHSP18.2pro (heat inducible promoter from Arabidopsis), GmHSP17.3Bpro (Heat inducible promoter from Soybean), and AtHSFB1pro (heat inducible promoter from Arabidopsis).

One or more of the promoters used in the methods and compositions of the invention may be a PDF1 promoter, such as a GbPDF1 promoter from G. barbadense or a GhPDF1 promoter from G. hirsutum. The cotton PROTODERMAL FACTOR1 gene (PDF) is predominantly expressed in the epidermis of ovules and developing fibers during fiber initiation and early elongation (Deng et al., 2012). In cotton plants, GbPDF1 was found to be preferentially expressed during fiber initiation and elongation, with a highest accumulation in fiber cells five days post anthesis. GbPDF1 promoter::GUS constructs in transgenic cotton predominantly expressed in the epidermis of ovules and developing fibers. (Deng et al., 2012). A 236 basepair promoter fragment of GbPDF1 was shown to promote GbPDF1 transcription in cotton. The temporally preferential expression of GbPDF1 during fiber initiation and elongation makes GbPDF1 promoters useful for controlling expression of a differently expressed gene in the in vitro methods of cotton production disclosed herein. Table 2 provides two exemplary cotton PDF1 promoters, which include the 236 base pair promoter fragment, and the full CDS of GbPDF1 and GhPDF1.

Due to the tissue and temporal preferential expression of GhACT genes in plantae, their promoter sequences are useful as promoters for the differentially expressed genes used in the in vitro methods of cotton production disclosed herein. Exemplary promoter nucleic acid sequences of GhACT1 are provided in Table 2.

GhMYB109 is a cotton fiber specific R2R3 MYB gene. Reporter gene analysis showed that a 2-kb GhMYB109 promoter provided fiber-specific expression using a GUS staining construct. (Pu et al., 2008). Due to this fiber-specific expression, GhMYB109 promoters are useful as promoters for controlling expression of a differently expressed gene in the in vitro methods of cotton production disclosed herein. An exemplary GbMYB109 promoter is provided in Table 2.

G. hirsutum being fiber-specific promoter (GhSCFP) activates transcription in seed coat and fiber associated genes. (Yaqoob et al. 2020). Due to this preferential regulation of seed coat and fiber associated genes, GhSCFP promoters are useful as promoters for controlling expression of a differently expressed gene in the in vitro methods of cotton production disclosed herein. Exemplary GhSCFP promoters are provided in Table 2.

In certain aspects, the introduced genes

Exemplary promoters useful in the present invention include those as set forth in Table 2. In certain aspects, the promoter includes a nucleic acid sequence having at least 90%, 95%, 96%, 97%, 98%, or at least 99% sequence identity to anyone of SEQ ID NOS: 46-57, or one or more fragments thereof. Exemplary promoters may include derivative of anyone of SEQ ID NOS: 46-57, or one or more fragments thereof. Derivatives may include, one or more mutations, such as deletions, point mutations, restriction site alterations, nucleotide substitutions, additions and/or codon modifications. Derivatives may also include, for example, regulatory elements, and/or functional elements or modified functional elements.

TABLE 2

| | Corresponding |
|---|---|
| Description | SEQ ID NO |
| Exemplary D113 Promoter nucleic acid sequence | SEQ ID NO: 48 |
| Exemplary GbPDF1 Promoter (GbPDF1-1) nucleic acid sequence | SEQ ID NO: 49 |
| Exemplary GbPDF1 Promoter (GbPDF1-2) nucleic acid sequence | SEQ ID NO: 50 |
| Exemplary GhMYB109 promoter nucleic acid sequence | SEQ ID NO: 51 |
| Exemplary S7 (Stunt 7) promoter nucleic acid sequence derived | SEQ ID NO: 52 |
| from subterranean clover stunt virus DNA segment 7, complete | |
| CDS (GenBank: AY159024.1) | |
| Exemplary Seed coat-specific FBP7 (floral binding protein 7) | SEQ ID NO: 53 |
| promoter nucleic acid sequence | |
| Exemplary TA29 promoter nucleic acid sequence | SEQ ID NO: 54 |
| Exemplary CaMV 35S promoter nucleic acid sequence | SEQ ID NO: 55 |
| Exemplary GhSCFP promoter nucleic acid sequence | SEQ ID NO: 56 |
| G. hirsutum cultivar Handan 5833 fiber-specific | SEQ ID NO: 57 |
| protein (SCFP) gene, including promoter region | |
| (region 1 . . . 633) and 5′ UTR (GenBank: GQ411495.1) | |
| AtHSP 18.2pro - inducible heat shock promoter from Arabidopsis. | SEQ ID NO: 58 |
| GmHSP17.3Bpro - inducible heat shock promoter from soy bean. | SEQ ID NO: 59 |
| AtHSFB1pro - inducible heat shock promoter from Arabidopsis. | SEQ ID NO: 60 |
| GhUbi14pro - constitutive endogenous promoter from cotton | SEQ ID NO: 61 |
| ubiquitin gene. | |

In certain aspects, the promoter includes a nucleic acid sequence having at least 90%, 95%, 96%, 97%, 98%, or at least 99% sequence identity to anyone of the sequences listed in Table 2, and/or one or more fragments thereof. Exemplary promoters may include derivative of anyone of SEQ ID NOS: 1, 3, 5-7, 9-10, 12-30, 33-36, and 38-45, or one or more fragments thereof. Derivatives may include, one or more mutations, such as deletions, point mutations, restriction site alterations, nucleotide substitutions, additions and/or codon modifications. Derivatives may also include, for example, regulatory elements, and/or functional elements or modified functional elements.

TABLE 3

| | SEQ | |
|---|---|---|
| Name | ID NO | Description |
| GVG_dex_inducible_pro | SEQ ID | Exemplary synthetic |
| | NO: 62 | transcription factor and |
| | | promoter fusion. Includes a |
| | | Dexamethazone inducible |
| | | promoter. |
| LhGR_dex_inducible_pro | SEQ ID | Exemplary synthetic |
| | NO: 63 | transcription factor and |
| | | promoter fusion. Includes a |
| | | Dexamethazone inducible |
| | | promoter. |
| RUBY | SEQ ID | Genes sufficient to enact |
| | NO: 64 | betailin synthesis, fused with |
| | | cleavable peptide linker P2a. |
| mas_pro::WUS::mas_term | SEQ ID | WUS from Arabidopsis under |
| | NO: 65 | the constitutive mas |
| | | promoter/terminator from |
| | | agrobacterium, to enforce stem |
| | | cell identity and promote |
| | | sensitivity to other treatments |
| AtHSP18.2pro::GaMYB2:35S | SEQ ID | Trichome initiation gene MYB2 |
| | NO: 66 | from G. arborium fused to the |
| | | heat inducible promoter from |
| | | Arabidopsis |
| AtHSP18.2pro::GhMYB25- | SEQ ID | Fiber initiation gene |
| like | NO: 67 | MYB25-like from G. hirsutum |
| | | fused to the heat inducible |
| | | promoter from Arabidopsis |
| GhSCFP_pro::GhFP1 | SEQ ID | Fiber specific expression from |
| | NO: 68 | SCFP gene driving the fiber |
| | | promoter gene FP1 from G. |
| | | hirsutum |
| 35S:nptII_2a_DsRED2:nos | SEQ ID | nptII gene translationally fused |
| | NO: 69 | to DSRED2 reporter with P2A |
| | | self-cleaving linker |

In certain aspects, a fusion or chimeric gene includes a nucleic acid sequence having at least 90%, 95%, 96%, 97%, 98%, or at least 99% sequence identity to anyone of the genes listed in Table 2, one or more fragments thereof, or an endogenous promoter sequence for such a gene. Derivatives may include, one or more mutations, such as deletions, point mutations, restriction site alterations, nucleotide substitutions, additions and/or codon modifications. Derivatives may also include, for example, regulatory elements, and/or functional elements or modified functional elements.

In certain aspects, modifying the cotton cell may involve the use of a vector. Vectors can be used to produce, transfer or manipulate transgenes, chimeric genes, and/or genetic constructs. A vector is a nucleic acid molecule, preferably a DNA molecule derived, for example, from a plasmid, bacteriophage or plant virus, into which a nucleic acid sequence may be inserted into a cotton cell plant, explant or cell. A vector may include one or more unique restriction sites, capable of autonomous replication in a selected cotton cell, tissue, explant, or plant, or integrated into the genome of a cotton cell, plant, or explant such that the cloned sequence is reproducible. The vector may be an autonomously replicating vector, which exists as an extrachromosomal entity, its replication independent of chromosomal replication, for example, a linear or closed circular plasmid, an extrachromosomal element, a mini-chromosome, or an artificial chromosome. The vector may be one which, when introduced into a cell, is integrated into the genome of the recipient cell and replicated together with the chromosome(s) into which it has been integrated.

A vector system may include a single vector or plasmid, or two or more vectors or plasmids, which contain the DNA to be introduced into the genome of a cell. A vector may also comprise a selection marker, for example, an antibiotic resistance gene that can be used for selection of suitable transformants.

In certain aspects, a vector is used to introduce the gene of interest into cotton cells. The cells may be obtained or derived from the tissue from any meristematic part of a cotton plant or explant, including apical meristems, cotyledons, young leaves, hypocotyls, ovules, ovule epidermal cells, stems, mature leaves, flower, flower stalks, floral whorls, roots, bulbs, germinated seeds, somatic and zygotic embryo, and/or cambial meristematic cells (CMC).

Alternatively, the vector is used to introduce the gene of interest into a cotton plant, cotton seed, cotton explant, and/or cotton plant tissue. After introduction, cotton plants, explants, and/or cotton plant tissue can be grown. Once grown, cotton cells can be selected. In certain aspects, after introduction, cotton plants are grown for one or more generations before cells are selected. The cotton plants can be backcrossed or crossed with other cotton plants to introduce desirable genetic backgrounds before the cells are selected.

In certain aspects, cotton cells and/or plants can be modified with an induced mutation. An induced mutation is an artificially induced genetic variation, for example, using chemical, radiation or biologically-based mutagenesis. The resulting mutations may include nonsense mutations, frameshift mutations, additions deletions, insertional mutations or splice-site variants. The mutations may modulate the activity of the selected gene. In certain aspects, the cotton cells used in the methods for producing cotton fiber in vitro may include the use of cotton cells that have a differently expressed gene (DEG). A differently expressed gene may have increased or decreased expression when compared to an endogenous and/or wild type gene. In certain aspects, the an endogenous and/or wild type gene may have no naturally occurring expression. A DEG may also have a different spatial and/or temporal expression pattern when compared to an endogenous and/or wild type gene. The DEG can be synthesized to include a promoter that assures the DEG is expressed at a certain level, under specific growth conditions, in certain tissues and tissue types, and/or under specified spatial and/or temporal constraints.

Cotton cells or plants of the present disclosure can be subject to a mutagenic process to give rise the DEG. This process can occur in vitro and without ever growing a whole cotton plant with the DEG.

Mutagenesis can be achieved by radiation and/or chemical means, including EMS or sodium azide treatment of seed, or gamma irradiation. Chemical mutagenesis favors nucleotide substitutions rather than deletions. Heavy ion beam (HIB) irradiation is a known technique for mutagenesis. Ion beam irradiation has two physical factors, the dose (gy) and LET (linear energy transfer, keV/um) for biological effects that determine the level of DNA damage and the size of any DNA deletion(s), and these can be adjusted according to change the extent of mutagenesis.

Biological agents can also be used to create site-specific mutations in cotton cells. These agents may include enzymes that cause double stranded breaks in DNA, which stimulate endogenous repair mechanisms. These enzymes include endonucleases, zinc finger nucleases, transposases and site-specific recombinases.

Isolation of cotton cells or plants with a selected gene may be achieved by screening mutagenized cotton plants or cells. For example, a mutagenized population of cotton plants may be screened directly for a particular genotype or indirectly by screening for a desired phenotype. Screening directly for the genotype may include assaying for the presence of mutations, e.g., using PCR- or sequencing-based assays.

In certain aspects, cotton cells are selected from cotton plants produced using the process known as TILLING (Targeting Induced Local Lesions IN Genomes). In a first step, introduced mutations are induced in a population of plants by treating seeds or pollen with a chemical or radiation mutagen, and then advancing plants to a generation where mutations will be stably inherited, typically an M2 generation where homozygotes may be identified. DNA is extracted, and seeds are stored from all members of the population to create a resource that can be accessed repeatedly over time. For a TILLING assay, PCR primers are designed to specifically amplify a single gene target of interest. PCR products from pooled DNA of multiple individuals are amplified using the primers. These PCR products are denatured and reannealed to allow the formation of mismatched base pairs. Mismatches, or heteroduplexes, represent both naturally occurring single nucleotide polymorphisms (SNPs) (i.e., several plants from the population are likely to carry the same polymorphism) and induced SNPs (i.e., only rare individual plants are likely to display the mutation). After heteroduplex formation, the use of an endonuclease that cleaves mismatched DNA is the key to discovering novel SNPs within a TILLING population.

In some embodiments, the cotton cells, plants, and/or explants (or engineered cotton) described herein can be derived from a Gossypium species. The Gossypium species can be selected from the group consisting of G. arboreum, G. anomalum, G. armourianum, G. klotzchianum, and G. raimondii. The cotton (or engineered cotton) can be derived from a Gossypium species selected from the group consisting of G. hirsutum, G. arboreum, G. barbadense, G. anomalum, G. armourianum, G. klotzchianum, and G. raimondii. The cotton (or engineered cotton) can be Gossypium hirsutum, Gossypium barbadense, Gossypium arboretum, Gossypium herbaceum, or another species of cotton.

Returning to FIG. 1, a bioreactor is inoculated 105 with a small number of the modified and/or selected cotton cells. The bioreactor may be inoculated with a small number of cotton ovule cells, which may include ovule epidermal cells. Generally, the bioreactor will be inoculated with a small number of cotton cells from a proliferating cell aggregate. As shown in Example 4, milligram quantities of cotton cells from a proliferating cell aggregate are sufficient to eventually inoculate a bioreactor.

Inoculating 105 may include preparing a growth medium in a vessel, such as a flask or plate, and introducing a small number of cotton cells from a proliferating cell aggregate into the medium. The vessel may then be left for inoculum growth. Alternatively, inoculum growth may occur inside the bioreactor.

The Inventors found that, surprisingly, inoculum growth under dark conditions provided superior growth. The vessel may be shaken or agitated during inoculum growth, for example, at a rate of 80-180 rpm. Preferably, inoculum growth occurs at a temperature of about 30° C. to about 35° C. Preferably, the medium is a solution that comprises plant hormones, plant growth regulators, and/or sucrose and/or glucose. Inoculum growth generally takes about 16 days, but may be more or less as desired or due to conditions or individual cotton cell lines.

The inoculum may be a cell suspension in a liquid or semi-solid medium. The suspension may be optionally homogenized to provide a fine cell suspension culture. The present Inventors discovered that a homogenous cell suspension can provide more reproducible and reliable results when inoculating a bioreactor.

Homogenizing may include any methods known in the art, including one or more of subculturing the suspension, filtering, pipetting/decanting, and/or addition of a low concentration of pectinase.

The resulting inoculum is then introduced into a bioreactor. Alternatively, the resulting inoculum can be preserved, e.g., by freezing, for later use in inoculating a bioreactor. The inoculum or homogenous cell suspension, which includes cells that include one or more selected genes of interest, may be cryopreserved indefinitely, for example, in liquid nitrogen. This generally requires suspending cells from the inoculum/homogenous cell suspension in a cryoprotectant solution, for example a solution of glycerol and sucrose. The cryoprotectant solution can be supplement, for example, using proline. Cryopreserved cells can be recovered, for example, using a recovery media, before their use in inoculating a bioreactor.

The proliferating cell aggregate may be a callus. Preferably, the proliferating cell aggregate is a friable callus, which is not sticky or soft, but is also not so hard or dense that it cannot be physically broken or crumbled. A friable callus thus differs “a hard callus”, which is compact and brittle, and thus not amenable to being broken or crumbled. The Inventors discovered that a friable callus allows for simple mechanical manipulation to easily disassociate individual cells from the friable callus for use in inoculating 105 a bioreactor and/or preparing an inoculum.

After inoculating 105, the method 101 requires multiplying 107 the cells in the bioreactor. This phase generally lasts for between 5 and 12 days, with duplication for the cotton cells taking approximately 1 to 3 days depending on cotton lineage. The cells may be duplicated, for example, by culturing the cells in a cell culture medium.

The multiplied cells are then elongated 109 to produce cotton fibers. This may include using an elongation medium to induce elongation in the multiplied cells. In certain aspects, the elongation medium facilitates release of a phenolic compound from a vacuole of an elongated cotton cell. The elongated cells may include cotton pre-fibers, which will mature into cotton fibers.

In certain aspects, a semi-solid elongation medium is used to elongate the cotton cells. The Inventors made the surprising discovery that superior results are achieved when using a semi-solid medium as opposed to a liquid medium.

Optionally, after elongation, the elongated cotton cells are separated from any non-elongated cotton cells. The non-elongated cotton cells will not mature into cotton fibers. However, they may be recycled and used in subsequent iterations of the method. Separating the elongated cotton cells from the non-elongated cells may include one or more of filtering, sieving, decanting, and centrifuging the cells.

Once separated, the elongated cotton cells, which at this point may have cotton pre-fibers, are matured. Maturing the cells may include the use of a maturation medium. During maturation, sugars are combined in the cells to produce cellulose, which is the main component of cotton fiber (natural glucose polymerization) that occurs inside the cell forming a secondary wall. The cotton pre-fibers increase in number, density, and/or length.

After maturation, cotton fiber harvested 111 from the cotton cells, by for example, separating the fibers from the cells in a solution/buffer. The harvested cotton fiber is then dried to a moisture content of less than 5% by, for example, passing air through the cotton fiber.

Thus, the method 101 can produce cotton fibers from cotton cells without growing cotton plants. These methods allow quick and efficient cultivation of cotton fiber in a controlled environment.

The method 101 may also include preparing a friable callus. A friable callus can be made, for example, by obtaining cells from a cotton explant and contacting the cells with a callus induction medium. Surprisingly, the Inventors discovered that tissue from any meristematic part of a cotton plant can be used to produce a friable callus. Thus, the cells from the cotton explant can from cotton apical meristems, cotyledons, young leaves, hypocotyls, ovules, ovule epidermal cells, stems, mature leaves, flower, flower stalks, floral whorls, roots, bulbs, germinated seeds, somatic and zygotic embryo, and/or cambial meristematic cells (CMC).

Preparing a friable callus may include contacting the cells of a cotton explant with a callus induction medium. The callus induction medium may facilitate the division of at least a subset of cells of a plant explant. Using the callus induction medium results in dedifferentiated cell masses. The cells in these masses can be subsequently cultured, which may include the use of a callus growth medium.

Certain aspects of the invention require the use of plant hormone(s) and/or growth regulator(s) (including auxins, gibberilins, etc.). The hormones/regulators can be used, for example, in the mediums described herein for culturing cotton cells. Plant hormones and/or growth regulators (including auxins, gibberilins, etc.) can be derived from naturally occurring sources, synthetically produced, or semi-synthetically produced, i.e. starting from naturally derived starting materials then synthetically modifying said materials. These modifications can be conducted using conventional methods as envisioned by a skilled worker. The following references include plant hormones and/or growth regulators (including auxins, gibberilins, etc.) for plant cell composition as described hereinbelow or described anywhere else herein: Gaspar et al. In Vitro Cell. Dev. Biol-Plant, 32, 272-289, October-December 1996 and Zhang et al. Journal of Integrative Agriculture, 2017, 16(8): 1720-1729; the contents of each of which (particularly, all the plant hormones and/or plant growth regulators) are incorporated by reference herein. In particular, one of skill in the art will understand that certain gibberilins are capable of facilitating plant cell elongation.

In some aspects, plant hormones and/or growth regulators used in the present invention are exemplified by those in Table A.

TABLE A
Exemplary plant hormones or plant growth regulators
and exemplary applications in plant cell engineering.

| | | | | Fiber | | |
|---|---|---|---|---|---|---|
| | | Callus | | initiation/ | Cell wall | Other |
| Name | Abbreviation | induction | Multiplication | elongation | thickening | applications |
| indole acetic acid | IAA | Y | Y | Y | Y | |
| indole butyric acid | IBA | Y | Y | Y | Y | |
| 2,4- | 2,4 D | Y | Y | Y | Y | |
| dichlorophenoxyacetic | | | | | | |
| acid | | | | | | |
| naphthaleneacetic acid | NAA | Y | Y | Y | Y | |
| para-chlorophenoxyacetic | pCPA | Y | Y | Y | Y | |
| acid | | | | | | |
| β-naphthoxyacetic acid | NOA | Y | Y | Y | Y | |
| 2-benzothiazole acetic | BTOA | Y | Y | Y | Y | |
| acid | | | | | | |
| picloram | PIC | Y | Y | Y | Y | |
| 2,4,5,- | 2,4,5-T | Y | Y | Y | Y | |
| trichlorophenoxyacetic | | | | | | |
| acid | | | | | | |
| phenylacetic acid | PAA | Y | Y | Y | Y | |
| kinetin | KIN | Y | Y | Inhibitor | ND | |
| 6-benzylaminopurine | 6BA | Y | Y | Inhibitor | ND | |
| N6-(2-isopentenyl) | 2iP | Y | Y | Inhibitor | ND | |
| adenine | | | | | | |
| zeatin | ZEA | Y | Y | Inhibitor | ND | |
| gibberellin A1 | GA1 | ND | ND | Y | ND | Control fiber |
| gibberellic acid | GA3 | ND | ND | Y | ND | |
| gibberellin A4 | GA4 | ND | ND | Y | ND | strength, |
| gibberellin A7 | GA7 | ND | ND | Y | ND | micronaire |
| | | | | | | and |
| | | | | | | maturation |
| ethylene | — | ND | ND | Y | ND | |
| brassinolide | BR | ND | ND | Y | Y | |
| jasmonic acid | JA | ND | ND | Y | ND | |

“Y” indicates that the corresponding plant hormone or plant growth regulator in the row can be used for the application indicated in the column heading.
“Inhibitor” indicates that the corresponding plant hormone or plant growth regulator in the row can be used for inhibiting the activity indicated in the column heading.
“ND” indicates that effect(s) of the corresponding plant hormone or plant growth regulator for the application indicated in the column heading is not yet determined (at least to some extent).

In certain aspects, the invention uses an induction medium or callus induction medium. The callus induction medium described herein can be configured to facilitate division of at least a subset of cells of a plant explant. For example, the callus induction medium can facilitate or promote induction of a cotton plant callus. The callus induction medium can comprise a diluted basal medium (i.e., from 1:1.5 to 1:5, from 1:1.5 to 1:4, from 1:1.5 to 1:3, etc.). The callus induction medium can comprise one or more salts, macronutrients, micronutrients, organic molecules, and/or hormones (such as those that can facilitate or promote induction). The callus induction medium can be a liquid at about 25° C. Alternatively, the callus induction medium can be not a liquid at a specified temperature. In some embodiments, the callus induction medium is not a liquid at about 25° C. In some embodiments, the callus induction medium can be a semi-solid medium (such as gelled) at 25° C.

Non-limiting examples of a semi-solid medium include soft agar, soft agarose, soft methylcellulose, xantham gum, gellan gum, carrageenan, isabgol, guar gum, other soft polymeric gels, or any other gelling agent known in the art. The callus induction medium can comprise agar. In some embodiments, the callus induction medium can be agar-free. In some embodiments, the callus induction medium is free of any gelling agent. In some embodiments, the callus induction medium that is agar- or gelling agent-free can be a liquid. In some embodiments, the callus induction medium that is agar- or gelling agent-free can be a solid. In some embodiments, the callus induction medium that is agar-free can be a gel. In some embodiments, the callus induction medium that is agar-free can comprise an agar-substitute. In some embodiments, the callus induction medium can have a pH. The pH of the callus induction medium can be appropriate for induction of a plant callus. In some embodiments, the pH of the callus induction medium can be optimized for induction of a plant callus. In some embodiments, the pH of the callus induction medium can be from 5.3 to 6.3. In some embodiments, the pH of the callus induction medium can be, or be about, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, or 6.9, or a range between any two foregoing values.

The present disclosure includes callus mediums or callus growth mediums, and their use in the in vitro methods for producing cotton. The callus growth medium described herein can facilitate or promote growth of a plant callus and/or produce a proliferating cell aggregate. The callus growth medium can be a gel medium, and in some embodiments, can comprise agar and/or another gelling agent and a mixture of macronutrients and micronutrients for the plant type of the plant callus. In some cases, the callus medium can be enriched with nitrogen, phosphorus, or potassium. In some cases, a callus growth medium can be a liquid medium. In some embodiments, the callus growth medium can comprise at least one plant hormone or growth regulator (including auxins, gibberilins, etc.), or at least two plant hormones or growth regulators, or at least three plant hormones or growth regulators, or at least four plant hormones or growth regulators, or at least five plant hormones or growth regulators, or at least six plant hormones or growth regulators, or at least seven plant hormones or growth regulators, or at least eight plant hormones or growth regulators. The at least one plant hormone or plant growth regulator (or at least two, at least three, at least four, at least five, or at least six plant hormones or plant growth regulators) (including auxins, gibberilins, etc.) can be any one or combination selected from the group consisting of indole acetic acid (IAA), Indoyl-3-acrylic acid, 4-Cl-Indoyl-3-acetic acid, Indoyl-3-acetylaspartate, indole-3-acetaldehyde, indole-3-acetonitrile, indole-3-lactic acid, indole-3-propionic acid, indole-3-pyruvic acid, indole butyric acid (IBA), 2,4-dichlorophenoxyacetic acid (2,4 D), tryptophan, phenylacetic acid (PAA), Glucobrassicin, naphthaleneacetic acid (NAA), picloram (PIC), Dicamba, ethylene, para-chlorophenoxyacetic acid (pCPA), β-naphthoxyacetic acid (NOA), benzo(b)selenienyl-3 acetic acid, 2-benzothiazole acetic acid (BTOA), N6-(2-isopentenyl) adenine (2iP), zeatin (ZEA), dihydro-Zeatin, Zeatin riboside, kinetin (KIN), 6-(benzyladenine)-9-(2-tetrahydropyranyl)-9H-purine, 2,4,5,-trichlorophenoxyacetic acid (2,4,5-T), 6-benzylaminopurine (6BA), 1,3-diphenylurea, N-(2-chloro-4-pyridyl)-N′-phenylurea, (2,6-dichloro-4-pyridyl)-N′-phenylurea, N-phenyl-N′-1,2,3-thiadiazol-5-ylurea, gibberellin A₅, gibberellin A1 (GA1), gibberellic acid (GA3), gibberellin A4 (GA4), gibberellin A7 (GA7), brassinolide (BR), jasmonic acid (JA), gibberellin A₈, gibberellin A₃₂, gibberellin A₉, 15-β-OH-gibberellin A₃, 15-β-OH-gibberellin A₅, 12-β-OH-gibberellin A₅, 12-α-gibberellin A₅, salicylic acid, (−) jasmonic acid, (+)-7-iso-jasmonic acid, putrescine, spermidine, spermine, oligosaccharins, and stigmasterol. The at least one plant hormone or plant growth regulator (or at least two, at least three, at least four, at least five, or at least six plant hormones or plant growth regulators) (including auxins, gibberilins, etc.) can be any one or combination selected from the group consisting of indoyl-3-acetic acid, indoyl-3-acrylic acid, indoyl-3-butyric acid, 4-Cl-Indoyl-3-acetic acid, Indoyl-3-acetylaspartate, indole-3-acetaldehyde, indole-3-acetonitrile, indole-3-lactic acid, indole-3-propionic acid, indole-3-pyruvic acid, tryptophan, phenylacetic acid, Glucobrassicin, 2,4-Dichlorophenyoxyacetic acid, 1-naphthaleneacetic acid, Dicamba, Pichloram, ethylene, benzo(b)selenienyl-3 acetic acid, trans-Zeatin, N⁶-(2-isopentyl) adenine, dihydro-Zeatin, Zeatin riboside, Kinetin, benzylamide, 6-(benzyladenine)-9-(2-tetrahydropyranyl)-9H-purine, 1,3-diphenylurea, N-(2-chloro-4-pyridyl)-N′-phenylurea, (2,6-dichloro-4-pyridyl)-N′-phenylurea, N-phenyl-N′-1,2,3-thiadiazol-5-ylurea, Gibberellin A₁, Gibberellin A₃, Gibberellin A₄, Gibberellin A₅, Gibberellin A₇, Gibberellin A₈, Gibberellin A₃₂, Gibberellin A₉, 15-β-OH Gibberellin A₃, 15-β-OH Gibberellin A₅, 12-β-OH Gibberellin A₅, 12-α-Gibberellin A₅, salicylic acid, jasmonic acid, (−) jasmonic acid, (+)-7-iso-jasmonicacid, putrescine, spermidine, spermine, oligosaccharins, brassinolide, and stigmasterol. The at least one plant hormone or plant growth regulator (or at least two, at least three, at least four, at least five, or at least six plant hormones or plant growth regulators) (including auxins, gibberilins, etc.) can be any one or combination selected from the group consisting of indole acetic acid (IAA), indole butyric acid (IBA), 2,4-dichlorophenoxyacetic acid (2,4 D), naphthaleneacetic acid (NAA), para-chlorophenoxyacetic acid (pCPA), β-naphthoxyacetic acid (NOA), 2-benzothiazole acetic acid (BTOA), picloram (PIC), 2,4,5,-trichlorophenoxyacetic acid (2,4,5-T), phenylacetic acid (PAA), kinetin (KIN), 6-benzylaminopurine (6BA), N6-(2-isopentenyl) adenine (2iP), zeatin (ZEA), gibberellin A1 (GA1), gibberellic acid (GA3), gibberellin A4 (GA4), gibberellin A7 (GA7), ethylene, brassinolide (BR), and jasmonic acid (JA).

In certain aspects, the callus growth medium can be a liquid at about 25° C. In some embodiments, the callus growth medium can be not a liquid at about 25° C. In some embodiments, the callus growth medium can be a semi-solid medium (such as gelled) at 25° C. Non-limiting examples of a semi-solid medium include soft agar, soft agarose, soft methylcellulose, xantham gum, gellan gum, carrageenan, isabgol, guar gum, other soft polymeric gels, or any other gelling agent known in the art. In some embodiments, the callus growth medium can comprise agar. In some embodiments, the callus growth medium can be agar-free. In some embodiments, the callus growth medium is free of any gelling agent. In some embodiments, the callus growth medium that is agar- or gelling agent-free can be a liquid. In some embodiments, the callus growth medium that is agar- or gelling agent-free can be a solid. In some embodiments, the callus growth medium that is agar-free can be a gel. In some embodiments, the callus growth medium that is agar-free can comprise an agar-substitute.

In some embodiments, the callus growth medium can have a pH. The pH of the callus growth medium can be appropriate for growing a plant callus and/or producing a proliferating cell aggregate. In some embodiments, the pH of the callus growth medium can be optimized for growing a plant callus and/or producing a proliferating cell aggregate. In some embodiments, the pH of the callus growth medium can be from 5.3 to 6.3. In some embodiments, the pH of the callus growth medium can be, or be about, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, or 6.9, or a range between any two foregoing values.

The present invention includes cell culture mediums (e.g., a multiplication/duplication mediums), and their use in the in vitro methods for producing cotton described herein. In some embodiments, the cell culture medium described herein can facilitate or promote proliferation of a cell population, or a proliferating cell aggregate. The cell culture medium can comprise one or more salts, macronutrients, micronutrients, organic molecules, and/or hormones (such as those that can facilitate or promote proliferation). In some cases, the cell culture medium can be configured to proliferate a cell population, such as a proliferating cell aggregate. The cell culture medium can comprise an enzyme that can degrade a plant cell wall of a plant cell of a cell population, or a proliferating cell aggregate. In some embodiments, the enzyme can be a pectocellulolytic enzyme. In some embodiments, the enzyme can comprise cellulase, hemicellulose, cellulysin, or a combination thereof. In some embodiments, the cell culture medium can have a pH. The pH of the cell culture medium can be appropriate for culturing a cell population, or a proliferating cell aggregate.

In some embodiments, the pH of the cell culture medium can be optimized for culturing a cell population, such as a proliferating cell aggregate. In some embodiments, the pH of the cell culture medium can be optimized for cell division. In some embodiments, the pH of the cell culture medium can be from 5.3 to 6.3. In some embodiments, the pH of the cell culture medium can be, or be about, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, or 6.9, or a range between any two foregoing values. In some embodiments, the cell culture medium can have a different pH than a callus growth medium. In some embodiments, the cell culture medium can have a same pH as a callus growth medium. In some embodiments, the pH of the cell culture medium can differ from a pH of a callus growth medium by less than 0.1, less than 0.2, or less than 0.3 units. For example, the pH of a cell culture medium can differ from a pH of a callus growth medium by less than 0.2 units.

Preferably, a cell culture medium of the present disclosure includes one or more of MS, B5, glucose, sucrose, Kinetin, 2,4-dichlorophenoxyacetic acid (2,4-D), NAA, and coconut water. Preferably, the cell culture medium comprises 2,4-D. In certain aspects, the cell culture medium includes MS, B5, glucose/sucrose, and 2,4-D.

The present invention also includes recovery mediums, and their use in the in vitro methods for producing cotton fiber. A recovery medium can be used, for example, for recovery of cotton cell inoculum after cryopreservation. Some embodiments described herein are related to a recovery medium. In some embodiments, the recovery medium described herein can be a medium that can facilitate or promote recovery of cotton cells. The recovery medium can comprise one or more salts, macronutrients, micronutrients, organic molecules, and/or hormones that can facilitate or promote elongation.

The present invention includes elongation mediums, and their use in the in vitro methods for producing cotton fiber. The elongation mediums described herein can facilitate or promote elongation of cells capable of being elongated, for example, elongation of cotton cells. The elongation mediums described herein can comprise one or more salts, macronutrients, micronutrients, organic molecules, and/or hormones (such as those that can facilitate or promote elongation). In some embodiments, the elongation mediums can be configured to facilitate a release of a phenolic compound from a vacuole from a cotton cell. In some embodiments, the phenolic compound (such as O-diphenol) is configured to initiate fiber differentiation by inhibiting indoleacetic acid (IAA) oxidase and/or increase an intracellular auxin level. In some embodiments, the elongation medium can comprise at least one plant hormone or growth regulator (including auxins, gibberilins, etc.), or at least two plant hormones or growth regulators, or at least three plant hormones or growth regulators, or at least four plant hormones or growth regulators, or at least five plant hormones or growth regulators, or at least six plant hormones or growth regulators, or at least seven plant hormones or growth regulators, or at least eight plant hormones or growth regulators. The at least one plant hormone or plant growth regulator (or at least two, at least three, at least four, at least five, or at least six plant hormones or plant growth regulators) (including auxins, gibberilins, etc.) can be any one or combination selected from the group consisting of indole acetic acid (IAA), Indoyl-3-acrylic acid, 4-Cl-Indoyl-3-acetic acid, Indoyl-3-acetylaspartate, indole-3-acetaldehyde, indole-3-acetonitrile, indole-3-lactic acid, indole-3-propionic acid, indole-3-pyruvic acid, indole butyric acid (IBA), 2,4-dichlorophenoxyacetic acid (2,4 D), tryptophan, phenylacetic acid (PAA), Glucobrassicin, naphthaleneacetic acid (NAA), picloram (PIC), Dicamba, ethylene, parachlorophenoxyacetic acid (pCPA), β-naphthoxyacetic acid (NOA), benzo(b)selenienyl-3 acetic acid, 2-benzothiazole acetic acid (BTOA), N6-(2-isopentenyl) adenine (2iP), zeatin (ZEA), dihydro-Zeatin, Zeatin riboside, kinetin (KIN), 6-(benzyladenine)-9-(2-tetrahydropyranyl)-9H-purine, 2,4,5,-trichlorophenoxyacetic acid (2,4,5-T), 6-benzylaminopurine (6BA), 1,3-diphenylurea, N-(2-chloro-4-pyridyl)-N′-phenylurea, (2,6-dichloro-4-pyridyl)-N′-phenylurea, N-phenyl-N′-1,2,3-thiadiazol-5-ylurea, gibberellin A₅, gibberellin A1 (GA1), gibberellic acid (GA3), gibberellin A4 (GA4), gibberellin A7 (GA7), brassinolide (BR), jasmonic acid (JA), gibberellin A₈, gibberellin A₃₂, gibberellin A₉, 15-β-OH-gibberellin A₃, 15-β-OH-gibberellin A₅, 12-β-OH-gibberellin A₅, 12-α-gibberellin A₅, salicylic acid, (−) jasmonic acid, (+)-7-isojasmonic acid, putrescine, spermidine, spermine, oligosaccharins, and stigmasterol. The at least one plant hormone or plant growth regulator (or at least two, at least three, at least four, at least five, or at least six plant hormones or plant growth regulators) (including auxins, gibberilins, etc.) can be any one or combination selected from the group consisting of indoyl-3-acetic acid, indoyl-3-acrylic acid, indoyl-3-butyric acid, 4-Cl-Indoyl-3-acetic acid, Indoyl-3-acetylaspartate, indole-3-acetaldehyde, indole-3-acetonitrile, indole-3-lactic acid, indole-3-propionic acid, indole-3-pyruvic acid, tryptophan, phenylacetic acid, Glucobrassicin, 2,4-Dichlorophenyoxyacetic acid, 1-naphthaleneacetic acid, Dicamba, Pichloram, ethylene, benzo(b)selenienyl-3 acetic acid, trans-Zeatin, N⁶-(2-isopentyl) adenine, dihydro-Zeatin, Zeatin riboside, Kinetin, benzylamide, 6-(benzyladenine)-9-(2-tetrahydropyranyl)-9H-purine, 1,3-diphenylurea, N-(2-chloro-4-pyridyl)-N′-phenylurea, (2,6-dichloro-4-pyridyl)-N′-phenylurea, N-phenyl-N′-1,2,3-thiadiazol-5-ylurea, Gibberellin A₁, Gibberellin A₃, Gibberellin A₄, Gibberellin A₅, Gibberellin A₇, Gibberellin A₈, Gibberellin A₃₂, Gibberellin A₉, 15-β-OH-Gibberellin A₃, 15-β-OH-Gibberellin A₅, 12-β-OH-Gibberellin A₅, 12-α-Gibberellin A₅, salicylic acid, jasmonic acid, (−) jasmonic acid, (+)-7-iso-jasmonicacid, putrescine, spermidine, spermine, oligosaccharins, brassinolide, and stigmasterol. The at least one plant hormone or plant growth regulator (or at least two, at least three, at least four, at least five, or at least six plant hormones or plant growth regulators) (including auxins, gibberilins, etc.) can be any one or combination selected from the group consisting of indole acetic acid (IAA), indole butyric acid (IBA), 2,4-dichlorophenoxyacetic acid (2,4 D), naphthaleneacetic acid (NAA), para-chlorophenoxyacetic acid (pCPA), β-naphthoxyacetic acid (NOA), 2-benzothiazole acetic acid (BTOA), picloram (PIC), 2,4,5,-trichlorophenoxyacetic acid (2,4,5-T), phenylacetic acid (PAA), kinetin (KIN), 6-benzylaminopurine (6BA), N6-(2-isopentenyl) adenine (2iP), zeatin (ZEA), gibberellin A1 (GA1), gibberellic acid (GA3), gibberellin A4 (GA4), gibberellin A7 (GA7), ethylene, brassinolide (BR), and jasmonic acid (JA).

In certain aspects, the callus growth medium can be a liquid at about 25° C. In some embodiments, the callus growth medium can be not a liquid at about 25° C. In some embodiments, the callus growth medium can be a semi-solid medium (such as gelled) at 25° C. The present Inventors discovered that a semi-solid medium provides better results than a liquid medium. Non-limiting examples of a semi-solid medium include soft agar, soft agarose, soft methylcellulose, xantham gum, gellan gum, carrageenan, isabgol, guar gum, other soft polymeric gels, or any other gelling agent known in the art. In some embodiments, the callus growth medium can comprise agar. In some embodiments, the callus growth medium can be agar-free. In some embodiments, the callus growth medium is free of any gelling agent. In some embodiments, the callus growth medium that is agar- or gelling agent-free can be a liquid. In some embodiments, the callus growth medium that is agar- or gelling agent-free can be a solid. In some embodiments, the callus growth medium that is agar-free can be a gel. In some embodiments, the callus growth medium that is agar-free can comprise an agar-substitute.

In some embodiments, the elongation medium can have a pH. The pH of the elongation medium can be appropriate for producing/inducing an elongated cell, such as an elongated cotton cell or a plurality of elongated cotton cells. In some embodiments, the pH of the elongation medium can be optimized for cell elongation (such as cotton cell elongation). In some embodiments, the pH of the elongation medium can be from 5.3 to 6.3. In some embodiments, the pH of the elongation medium can be, or be about, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, or 6.9, or a range between any two foregoing values.

The present invention includes elongation mediums, and their use in the in vitro methods for producing cotton fiber. In some embodiments, the maturation mediums described herein can facilitate or promote maturation of cells, such as maturation of cotton cells. A maturation medium can comprise one or more salts, macronutrients, micronutrients, organic molecules, and/or hormones (such as those that can facilitate or promote maturation). In some embodiments, the maturation medium can comprise a maturation reagent. In some embodiments, the maturation reagent of the maturation medium can be a wall-regeneration reagent.

The present invention includes the use of proliferating cell aggregates, and their creation, for use in the in vitro methods of cotton fiber production. In some embodiments, the plant cell composition as described hereinbelow or described anywhere else herein can be derived from the proliferating cell aggregate. The proliferating cell aggregate can be an aggregate of plant cells that are proliferating. Proliferating cells in an aggregate can be attached or connected to each other, for example, via cell-to-cell interactions. The proliferating cell aggregate can be a friable callus is friable, which is not sticky or soft, but is also not so hard or dense that it cannot be physically broken or crumbled. A friable callus thus differs “a hard callus”, which is compact and brittle, and thus not amenable to being broken or crumbled. Preferably the callus is a friable callus. The present Inventors discovered that a friable callus can have individual cells dissociated from the callus using simple mechanical manipulation.

Proliferating cells can be of one type (a homogenous aggregate) or of two or more types (a heterogeneous aggregate). The proliferating cell aggregate can be a mixed aggregate (e.g., where cell types are mixed together), a clustering aggregate (e.g., where cells of different types are tending toward different parts of the aggregate), or a separating aggregate (where cells of different types are pulling apart from each other). Cells of the proliferating cell aggregate can divide at a rate greater than a cell division rate of remaining cells in said plant callus. In some embodiments, cells of the proliferating cell aggregate can divide at a rate that can be at least 2, 3, 4, 5, 6, 7, 8, 9, 10, 20, 30, 40, or 50 times greater than a cell division rate of plant callus cells.

The present invention includes the use of cells from a cotton plant cell callus and methods for preparing such a callus. The plant callus can be a growing mass of plant parenchyma cells. However, the Inventors discovered that, surprisingly, cells from any meristematic part of a cotton plant are sufficient for callus induction. Thus, the plant callus can be created using cells obtained or derived from cotton apical meristems, cotyledons, young leaves, hypocotyls, ovules, ovule epidermal cells, stems, mature leaves, flower, flower stalks, floral whorls, roots, bulbs, germinated seeds, somatic and zygotic embryo, and/or cambial meristematic cells (CMC). In some cases, the mass of plant parenchyma cells can be unorganized. The plant callus can be collected from cells covering the wound of a plant or plant part. Preferably, the plant callus is created by inducing a plant tissue sample (e.g., an explant) with a callus induction medium. In some cases, induction of an explant can occur after surface sterilization and plating onto a medium in vitro (e.g., in a closed culture vessel such as a Petri dish). Induction can comprise supplementing the medium with plant growth regulators, such as auxins, cytokinins, or gibberellins to initiate callus formation. Induction can be performed at a temperature of, or of about, 20° C., 25° C., 28° C., 30° C., 35° C., or 40° C., or a range between any two foregoing values.

Compositions comprising cotton plant cells are included in the present invention. The plant cell compositions described herein can be a final product of a method for preparation of cell bank stocks provided herein. The plant cell compositions can be compositions of engineered cells, or a compositions of wildtype cells. The plant cell compositions can be cell bank stocks. The plant cell compositions can comprise a plurality of plant cells obtained by growing a callus in a growth medium to produce a proliferating cell aggregate followed by culturing the proliferating cell aggregate.

The plant cell compositions described herein can be in a growth phase. The growth phase can comprise cell division, cell enlargement, and/or cell differentiation. The growth phase comprising cell division can be an exponential growth phase (e.g., dowaiting). In some embodiments, the exponential growth phase can occur as cells are mitotic. In some embodiments, during exponential growth, each generation of cells can be twice as numerous as the previous generation. In some embodiments, not all cells may survive in a given generation. In some embodiments, each generation of cells can be less than twice as numerous as the previous generation. In some embodiments, the exponential growth phase can be determined (e.g., quantified or identified) by a cell viability assay. In some embodiments, another aspect of the plant cell composition can be determined by a cell viability assay. In some embodiments, the cell viability assay can be an assay that can determine the ability of a cell to maintain or recover viability. In some embodiments, the cells of the plant cell composition can be assayed for their ability to divide or for active cell division. In some embodiments, the cell viability assay can be an ATP test, calcein AM, clonogenic assay, ethidium homodimer assay, Evans blue, fluorescein diacetate hydrolysis/propidium iodide staining (FDA/PI staining), flow cytometry, formazan-based assays (e.g., MTT or XTT), green fluorescent protein based assays, lactate dehydrogenase (LDH) based assays, methyl violet, neutral red uptake, propidium iodide, resazurin, trypan blue, or a TUNEL assay. In some embodiments, the cell viability assay can determine a cytoplasmic level of diphenol compounds in the plant cell composition.

Also provided herein are bioreactors configured to produce any one or more compositions associated with the in vitro production of fiber as disclosed herein

In some embodiments, a bioreactor can be configured to produce a cell bank stock. In some embodiments, a bioreactor can be configured to carry out a method for preparing a cell bank stock. In some such cases, a bioreactor can be configured to utilize components of a kit for preparation of a cell bank stock, such as a callus growth medium and/or a multiplication medium.

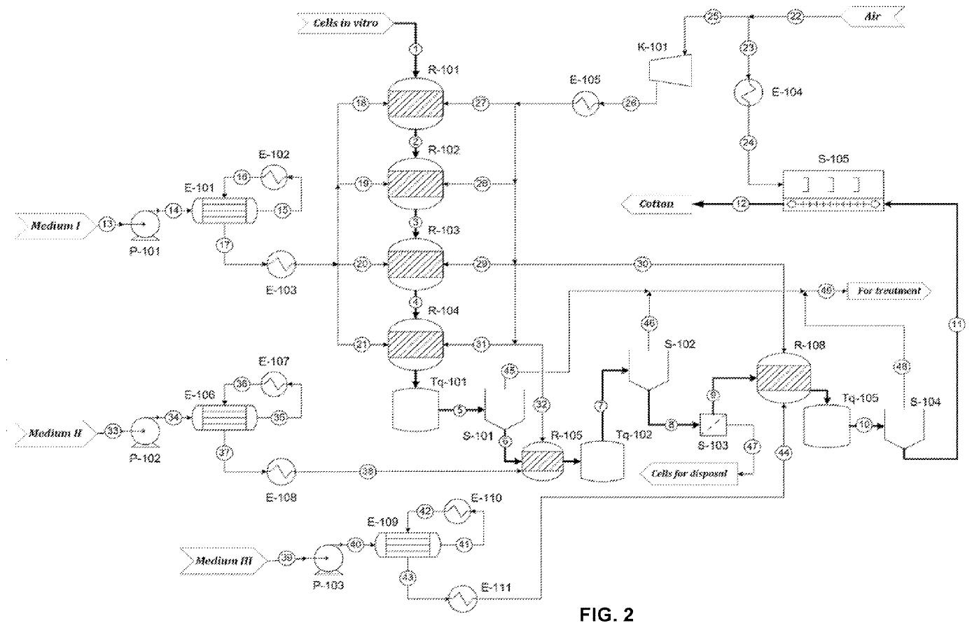

FIG. 2 provides a flow chart illustrating an example of different processes that can be performed by a bioreactor, and how these processes can be interconnected.

In some embodiments, a bioreactor can be configured to produce a cotton fiber. In some embodiments, a bioreactor can be configured to carry out a method for large scale cotton fiber production. In some embodiments, a bioreactor can be configured to carry out a method for rapid cotton fiber production. In some embodiments, a bioreactor can be configured to utilize components of a kit for large scale fiber production. In some embodiments, a bioreactor can be configured to utilize components of a kit for rapid fiber production.

In some embodiments, a bioreactor can be configured to produce engineered cotton. In some embodiments, a bioreactor can be configured to utilize components of a kit for production of engineered cotton, which can comprise elements of kits provided herein.

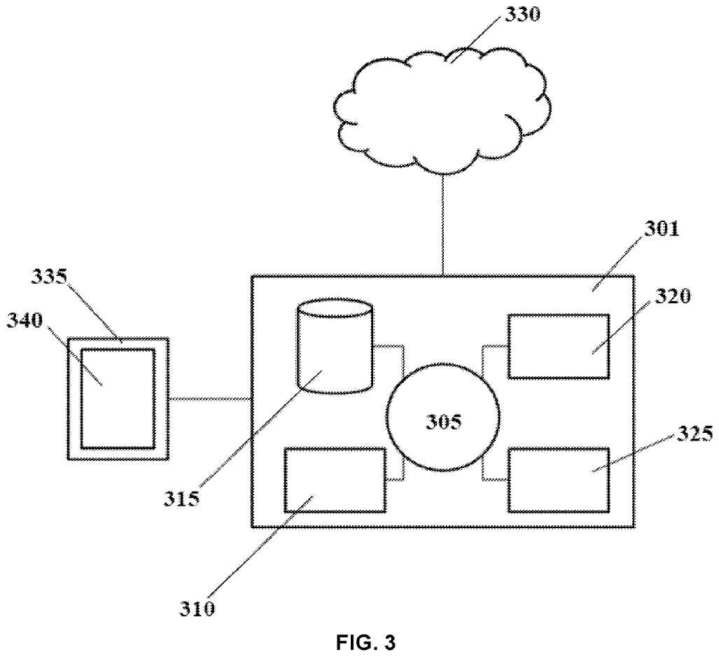

The present invention includes computer systems that are programmed to implement methods of the disclosure. FIG. 3 shows a computer system 301 that is programmed or otherwise configured to provide and/or implement instructions for or means of implementation of induction, callus growth, cell culture, elongation, or maturation. The computer system 301 can regulate various aspects of induction, callus growth, cell culture, elongation, or maturation of the present disclosure. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 201 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine-readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, instructions for or means of implementation of induction, callus growth, cell culture, elongation, or maturation. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, provide and/or execute instructions for or means of implementation of induction, callus growth, cell culture, elongation, or maturation.

The present invention is further described by the following non-limiting Examples.

### EXAMPLES

### Example 1: Preparation of a Plant Cell Composition

From a select plant (e.g., cotton), cells are isolated by placing sterilized explants from apical meristems, cotyledons, young leaves, hypocotyls, ovules, ovule epidermal cells, stems, mature leaves, flower, flower stalks, floral whorls, roots, bulbs, germinated seeds, somatic and zygotic embryo, and cambial meristematic cells (CMC) on a callus induction medium (e.g., a semi-solid basal salts medium) for induction. The dedifferentiated masses formed are conditioned by passing three up to five subculturing at intervals of 21-26 days on a callus growth medium (e.g., a semi-solid basal salts medium) for growth.

After cell culture stabilization, cells from a soft or friable callus are transferred into a liquid medium to form a suspension cell. Suspensions are sub-cultured at intervals of 15-20 days for homogenization to provide fine cell suspension culture, by filtering, pipetting/decantation, or by addition of a low concentration of pectinase. The homogeneous nature of cells in these cultures give rise to reproducible and reliable results.

### Example 2: Cryopreservation of Suspension-Cultured Cells

Cryopreservation techniques remove the need for frequent culturing and, thus, reduce the chance of microbial contamination. The protocol provided below allows the cryopreservation of over 100 cell lines simultaneously in a single day.

Suspension-cultured cells from Gossypium spp. and other species in exponentially growing phase are transferred to 15 ml tubes and centrifuged at 100×g for 1 min. Cell suspensions are handled using micropipettes with large orifice tips. The supernatant is removed, and cells are then suspended in cryoprotectant solution (LS: 2M glycerol, 0.4M sucrose) supplemented with up to 100 mM L-proline at the cell density of 10% (v/v), and incubated at room temperature for 0-120 minutes with and without shaking at 60 rpm. Aliquots (0.5 ml) of cell suspensions are dispensed into cryovials (Fisher Scientific). Cryovials containing cell suspension in LS are cooled to −35° C. at a rate of −0.5, −1, or −2° C. min⁻¹ using a programmable freezer. After reaching −35° C., cells are kept at −35° C. for 0, 30, or 60 minutes, and then plunged into liquid nitrogen.

In vitro dedifferentiated plant cell suspension cultures are more convenient for large-scale production, as they offer the advantage of a simplified model system for the study of plants. Cell suspension cultures contain a relatively homogeneous cell population, allowing rapid and uniform access to nutrition, precursors, growth hormones, and signal compounds for the cells.

### Example 3: Cell Recovery

The vials containing cryopreserved cells are transferred from the liquid nitrogen storage vessel into a Dewar flask containing liquid nitrogen. Each vial is transferred (one by one) to a clean 35-40° C. water bath and gently flipped several times until thawed (the last piece of ice disappears). Immediately, each vial is placed on ice again. Each vial is centrifuged at 100 g, at 4° C. for 1-2 min. The outside of each vial is wiped with 70% (vol/vol) ethanol and the supernatant from each vial is removed using a sterile Pasteur pipette. A sterile 3.5-ml transfer pipette is used to transfer two-thirds' volume of the cells by spreading or placing them as a few clusters onto the filter paper. The dish is closed and sealed with Parafilm.

The dish(es) is covered with one or two sheets of filter paper to reduce the light intensity then placed in the culture room in regular conditions (24-26° C.). After 2 days of recovery, a spatula (width of 4 mm) is used to collect some cell mass (about 100-200 mg FW) from the plate and place into a microtube for viability testing. The remaining cells are transferred with the upper filter paper to a fresh recovery dish containing recovery medium. The dishes are closed and sealed, covered with filter paper, and then returned to the culture room.

Depending on their growth rates, cells are allowed to grow for an additional number of days in the same culture room, in regular conditions (24-26° C.). When most of the filter paper is covered with a thick layer of cells, the cell mass is transferred to a fresh dish containing recovery medium without filter paper for a further 1-2 weeks under standard conditions (at this recovery stage, agarose may be replaced by agar or another gelling agent). After a recovery period of 3-9 weeks, cells are transferred to a liquid medium to initiate suspension culture.

### Example 4: Bioreactor Inoculation

For inoculum, the medium is prepared with deionized (DI) water to make a total volume of 200 mL (1 L flask) and sterilized through autoclaving at 121° C. for 15 minutes. After cooling to room temperature, plant growth regulators and amino acids are added using a 0.2 μm pore size membrane filter. Twenty grams of cells are inoculated and maintained in a shaker in dark at a temperature from about 30° C. to about 35° C. at 80 rpm and left for inoculum growth. After 16 days (7 days of LAG phase and 9 days of exponential phase), the suspension is sufficiently dense for feeding the bioreactors (Titer=100 g L⁻¹, comparable a thick applesauce with no visible free medium).

An illustrative schematic of the bioreactor can be found in FIG. 2. The bioreactor is fed with in vitro cells, with sterilized medium, and air compression. The bioreactors are connected to the controller prior to inoculation, to stabilize pH 5.8 (±0.2) and to control and calibrate the flow of 02. As illustrated in the flowchart in FIG. 2, the first vessel of the inoculum train occurs at a temperature from about 30° C. to about 35° C. with a 100 g L⁻¹ of cells at an exponential phase. In parallel, the sterilization of the culture medium occurs at approximately 125 to approximately 140° C. and returns (stream 16) to the heat exchanger (stream 13) to cooling the medium at a temperature from about 30° C. to about 35° C. (E-103). With this, the sterile medium is ready to feed the reactors of the multiplication area (reactors R-101 to R-104).

The air for cell oxygenation is also adjusted to the process temperature in the heat exchanger (E-105) and thus is split into four different streams (streams 27, 28, 29 and 31) that feed the inoculum train (reactors R-101 to R-104).

The multiplication occurs in a duration from 5 to 12 days for cells, and the duplication time is approximately 1 day to 3 days (depending on linage(s)). These times conclude when the cell amount increases, for example, 64 times. In the end, the content is loaded to the next reactor (R-102) and so on. The last reactor (R-104) has an adjacent lung tank, where after the reaction the contents are discharged in the batch feeding tank (Tq-101) with continuous output (stream 5). Thus, during the multiplication time of the R-104 reactor, the Tq-101 is continuously unloading the cells for the next stage, the separation, at a continuous flow rate.

Table B, below, provides experimental results showing the success of inoculating a bioreactor using cotton cells in accordance with the methods disclosed herein. Table B provides details regarding the cotton varieties from which the cells were obtained, the cell growth medium which was inoculated in the bioreactor, and other relevant conditions.

TABLE B

| | | | | | | | Preliminary |
|---|---|---|---|---|---|---|---|
| Variety | | | | | Testing | Growth | Growth |
| Name | Quantity | | Volume | | medium | Conditions | Result |
| PD 2164 | 40 | mg | 8 ml in | | 1xMS, 1xB5, | 30° C., dark, | poor |
| | | | a 25 ml | | 30 g/L glucose, | 180RPM | |
| | | | bottle | | 0.548 mg/L | | |
| PD 2164 | 37 | mg | 30 ml in | | Kinetin, | 30° C., dark, | poor |
| | | | a 125 ml | | 0.1 mg/L 2,4-D | 180RPM | |
| | | | flask | | | | |
| Acala MAXXA | 53 | mg | 8 | ml | 1xMS, 1xB5, | 30° C., dark, | poor |
| | | | | | 30 g/L glucose, | 180RPM | |
| | | | | | 0.548 mg/L | | |
| Acala MAXXA | 31 | mg | 30 | ml | Kinetin, | 30° C., dark, | poor |
| | | | | | 0.1 mg/L 2,4-D | 180RPM | |
| FJA | 48 | mg | 8 | ml | 1xMS, 1xB5, | 30° C., dark, | poor |
| | | | | | 30 g/L glucose, | 180RPM | |
| | | | | | 0.548 mg/L | | |
| FJA | 41 | mg | 30 | ml | Kinetin, | 30° C., dark, | poor |
| | | | | | 0.1 mg/L 2,4-D | 180RPM | |
| Pima S-7 | 34 | mg | 8 | ml | 1xMS, 1xB5, | 30° C., dark, | poor |
| | | | | | 30 g/L glucose, | 180RPM | |
| | | | | | 0.548 mg/L | | |
| Pima S-7 | 48 | mg | 30 | ml | Kinetin, | 30° C., dark, | poor |
| | | | | | 0.1 mg/L 2,4-D | 180RPM | |
| Pima S-7 | 91 | mg | 30 | ml | 1xMS, 1xB5, | 30° C., dark, | poor |
| | | | | | 30 g/L glucose, | 180RPM | |
| | | | | | 0.548 mg/L | | |
| | | | | | Kinetin | | |
| Pima S-7 | 110 | mg | 30 | ml | 1xMS, 1xB5, | 30° C., dark, | good |
| | | | | | 30 g/L glucose, | 180RPM | |
| | | | | | 0.1 mg/L 2,4-D | | |
| Pima S-7 | 82 | mg | 30 | ml | 1xMS, 1xB5, | 30° C., light, | poor |
| | | | | | 30 g/L glucose, | 180RPM | |
| | | | | | 0.548mg/L | | |
| | | | | | Kinetin, | | |
| | | | | | 0.1 mg/L 2,4-D | | |

| | | | | |
|---|---|---|---|---|
| Pima S-7 | 8 ml in | 1xMS, 1xB5, | 30° C., dark, | good |
| | a 50 ml | 30 g/L Sucrose | 180RPM | |
| | flask | 0.1mg/L | | |
| | | Kinetin, 1 mg/L | | |
| | | 2,4-D, 100 ml/L | | |
| | | coconut | | |
| | | water(B105) | | |
| Pima S-7 | 8 ml in | 1xMS, 1xB5, | 30° C., dark, | good |
| | a 50 ml | 20 g/L Sucrose, | 180RPM | |
| | flask | 0.1mg/L | | |
| | | Kinetin, 1 mg/L | | |
| | | 2,4-D, 100 ml/L | | |
| | | coconut | | |
| | | water(B50) | | |
| Pima S-7 | 8 ml in | 1xMS, 1xMS, | 30° C., dark, | good |
| | a 50 ml | 30 g/L Sucrose, | 180RPM | |
| | flask | 0.18 mg/L | | |
| | | NAA, 0.2 mg/L | | |

As shown in Table B, inoculating the bioreactor is far more efficient when done under dark conditions as opposed to light. Accordingly, the present disclosure provides methods of inoculating a bioreactor using a cotton cell culture, wherein the inoculation occurs under dark conditions.

As shown in Table B, the composition of the growth medium used when inoculating a bioreactor has an impact on cell growth. Thus, the present disclosure provides methods of inoculating a bioreactor with cotton cells, wherein the growth medium comprises plant hormones or growth regulators. As shown in Table B, when the growth medium included 2,4-dichlorophenoxyacetic acid (2,4-D), growth improved. Thus, the present disclosure provides methods of inoculating a bioreactor with cotton cells, wherein the growth medium comprises 2,4-dichlorophenoxyacetic acid (2,4-D).

As shown in Table B, the methods of the present disclosure allowed successful cell growth when inoculating a bioreactor with cells from all cotton varieties tested. Thus, the present disclosure provides methods of inoculating a bioreactor with any of the cotton varieties disclosed herein. In certain embodiments, the cotton cells used to inoculate a bioreactor in accordance with the methods disclosed herein are derived and/or obtained, in whole or in part, from at least one cotton plant of a variety selected from PAYMASTER HS26, PD 2164, SA 2413, SEALAND #1 (G.B.×G.H.), SOUTHLAND M1, STATION MILLER, TASHKENT 1, TIDEWATER 29 (G.B.×G.H.), TOOLE, WESTERN STORMPROOF, ACALA 5, ALLEN 33, CD3HCABCUH-1-89, DELTAPINE 14, DES 24, DES 56, DIXIE KING, FJA, M.U.8B UA 7-44, NC 88-95, PAYMASTER HS200, Pima S-7, Acala MAXXA, Coasland 320, or a progeny of any thereof. In certain embodiments, the cotton cells used to inoculate a bioreactor in accordance with the methods disclosed herein are derived and/or obtained, in whole or in part, from at least one cotton plant of a variety selected from PD 2164, Acala MAXXA, FJA, Pima S-7, or a progeny of any thereof.

As shown in Table B, surprisingly, cells from cotton variety Pima S-7 provided good growth when inoculating a bioreactor. This included when using milligram quantities of cotton cells to form an inoculum and across a range of growth mediums. Unexpectedly, Pima S-7 provided superior growth/inoculation compared to Acala MAXXA and FJA. Moreover, this superior growth occurred even when using the same growth medium. For example, as shown in Table B, Pima S-7 provided good growth, while Acala MAXXA and FJA showed poor growth when all were cultured using a growth medium with the same concentrations MS, B5, glucose, Kinetin, and 2,4-D. Accordingly, the present disclosure provides inoculating a bioreactor with cells derived and/or obtained, in whole or in part, from a cotton plant of the Pima S-7 variety, or a progeny thereof.

### Example 5: Elongation of Cells

For elongation, plant cells are separated from the medium using a decanter vessel (S-101) (stream 6) and the medium can be relocated for water treatment (stream 45), as illustrated in the flowchart in FIG. 2. The elongation growth medium is added to the reactors to sterilization by autoclaving at same conditions used in multiplication step and cooling at a temperature from about 30° C. to about 35° C. for cell differentiation.

Thus, the cells from the multiplication (stream 6) feed three elongation reactors (R-105, R-106, and R-107) are represented by the reactor block (R-105) in the flowchart in FIG. 2. Each reactor receives a third of the cells and the reaction volume comprises the cells (stream 6), medium (stream 38), and air (stream 32) flows.

### Example 6: Separation and Isolation of Elongated Cells

After elongation according to Example 5, 3 tanks (Tq-102, Tq-103, and Tq-104) are fed, which in the flowchart in FIG. 2 are represented only by block Tq-102. Each tank, with volume slightly larger than those of the reactors, receives the substantially same volume of the three reactors. The output of the elongation tanks (stream 7) is routed to the second decanter (S-102). The bottom product (stream 8), comprising elongated and unelongated cells, is routed to the sieve (S-103), while the medium (stream 46) is removed to the effluent treatment. The function of the sieve is to remove unelongated and smaller cells that are not pre-fibers. The sieve (S-103) retains the elongated cells (pre-fibers) and releases all nonelongated cells (which will not become cotton fibers).

### Example 7: Maturation and Drying of Cells

In the maturation stage, as well as in the multiplication and elongation stages, a sterilized medium is used. Maturation is recognized by secondary cell wall deposition. Sugars are combined to produce cellulose, which is the main component of cotton fiber (natural glucose polymerization) that occurs inside the cell forming the secondary wall. In this process, the density of pre-fiber increases from 1.05 to 1.55 g/ml, which is the density of cotton fiber.

After maturation time, the R-108 output is directed to the buffer tank Tq-105 (FIG. 2) to enable a continuous downstream process. In the sequence, the mid-fiber mixture (stream 10) is routed to the third decanter (S-104), where the cotton fibers (stream 11) are separated from the medium (stream 48). At this stage, the fibers produced have moisture content above acceptable level (10 to 20% in water mass). To reduce the moisture content, a drying process working with air is implemented. This air passes through the cotton fibers and part of the water is removed until a moisture content of at most 5% is reached.

### Example 8: Recycling

In some embodiments, a composition created via a method described herein can be recycled. For example, in such a case, after completion of a method or step of a method, an aliquot of a composition is reserved and re-introduced into an earlier step in a method. In some cases, an aliquot of cells unsuccessful in induction, growth, elongation, or maturation is reserved and re-introduced into an earlier step in a method.

### Example 9: Production of Cotton Fiber from Cotton Ovule Cells

Tables C, D, and E, below, show the results of growth and elongation of cotton cell cultures in accordance with the methods disclosed herein. For each growth result, cotton ovule cells, which may include ovule epidermal cells, were mechanically extracted from a cotton boll. The extracted cotton ovule cells were cultured, multiplied, and in some cases, elongated. Each table provides the genotype/cultivar and variety name from which the cotton cell cultures were originally obtained. The tables also provide the ovule location from which the cells were taken from a parental cotton plant for the cell cultures.

TABLE C
Cotton cell culture results from cells
cultured from an upper ovule location.

| | Ovule | |
|---|---|---|
| Variety Name | location | Growth Results |
| PAYMASTER HS26 | Upper | some |
| PD 2164 | Upper | excellent |
| SA 2413 | Upper | poor |
| SEALAND #1 (G.B. X G.H.) | Upper | some/fiber |
| SOUTHLAND M1 | Upper | good |
| STATION MILLER | Upper | some |
| TASHKENT 1 | Upper | some |
| TIDEWATER 29 | Upper | some |
| (G.B. X G.H.) | | |
| TOOLE | Upper | poor |
| WESTERN STORMPROOF | Upper | some |
| ACALA 5 | Upper | excellent/fiber |
| ALLEN 33 | Upper | some |
| CD3HCABCUH-1-89 | Upper | good |
| DELTAPINE 14 | Upper | some |
| DES 24 | Upper | some |
| DES 56 | Upper | poor |
| DIXIE KING | Upper | some |
| FJA | Upper | some |
| M.U.8B UA 7-44 | Upper | some |
| NC 88-95 | Upper | some |
| PAYMASTER HS200 | Upper | some |
| Pima S-7 | Upper | some |
| Acala MAXXA | Upper | some |
| Coasland 320 | Upper | poor |

TABLE D
Cotton cell culture results from cells
cultured from a middle ovule location.

| | Ovule | |
|---|---|---|
| Variety Name | location | Growth |
| PAYMASTER HS26 | Middle | some |
| PD 2164 | Middle | excellent |
| SA 2413 | Middle | some/fiber |
| SEALAND #1 (G.B. X G.H.) | Middle | some/fiber |
| SOUTHLAND M1 | Middle | good |
| STATION MILLER | Middle | some |
| TASHKENT 1 | Middle | some |
| TIDEWATER 29 | Middle | some |
| (G.B. X G.H.) | | |
| TOOLE | Middle | some/fiber |
| WESTERN STORMPROOF | Middle | some |
| ACALA 5 | Middle | some |
| ALLEN 33 | Middle | some |
| CD3HCABCUH-1-89 | Middle | good |
| DELTAPINE 14 | Middle | some |
| DES 24 | Middle | some |
| DES 56 | Middle | poor |
| DIXIE KING | Middle | some |
| FJA | Middle | excellent |
| M.U.8B UA 7-44 | Middle | some/fiber |
| NC 88-95 | Middle | some |
| PAYMASTER HS200 | Middle | some |
| Pima S-7 | Middle | some |
| Acala MAXXA | Middle | some |
| Coasland 320 | Middle | poor |

TABLE E
Cotton cell culture results from cells
cultured from a bottom ovule location.

| | Ovule | |
|---|---|---|
| Variety Name | location | Growth |
| PAYMASTER HS26 | Bottom | some |
| PD 2164 | Bottom | excellent |
| SA 2413 | Bottom | some |
| SEALAND #1 (G.B. X G.H.) | Bottom | poor |
| SOUTHLAND M1 | Bottom | excellent |
| STATION MILLER | Bottom | some |
| TASHKENT 1 | Bottom | good |
| TIDEWATER 29 | Bottom | some |
| (G.B. X G.H.) | | |
| TOOLE | Bottom | some/fiber |
| WESTERN STORMPROOF | Bottom | good |
| ACALA 5 | Bottom | excellent/fiber |
| ALLEN 33 | Bottom | some |
| CD3HCABCUH-1-89 | Bottom | excellent |
| DELTAPINE 14 | Bottom | some |
| DES 24 | Bottom | some |
| DES 56 | Bottom | poor |
| DIXIE KING | Bottom | some/fiber |
| FJA | Bottom | excellent |
| M.U.8B UA 7-44 | Bottom | some/fiber |
| NC 88-95 | Bottom | some |
| PAYMASTER HS200 | Bottom | good |
| Pima S-7 | Bottom | excellent |
| Acala MAXXA | Bottom | excellent |
| Coasland 320 | Bottom | poor |

As shown in Tables C, D, E, all varieties were successfully grown in accordance with the methods of the present disclosure. Thus, the present disclosure provides cotton, methods of growing cotton in accordance with any of the method/protocols provided herein, and persistent cell lines, wherein the cotton cells are derived and/or obtained, in whole or in part, which can be used across a range of cotton species and varietals. Accordingly, the in vitro methods of cotton production can use cotton cells derived from a cotton plant of any varietal, including one selected from PAYMASTER HS26, PD 2164, SA 2413, SEALAND #1 (G.B.×G.H.), SOUTHLAND M1, STATION MILLER, TASHKENT 1, TIDEWATER 29 (G.B.×G.H.), TOOLE, WESTERN STORMPROOF, ACALA 5, ALLEN 33, CD3HCABCUH-1-89, DELTAPINE 14, DES 24, DES 56, DIXIE KING, FJA, M.U.8B UA 7-44, NC 88-95, PAYMASTER HS200, Pima S-7, Acala MAXXA, Coasland 320, and or a progeny of any thereof.

As shown in Tables C, D, and E, certain varieties produced good or excellent growth. Thus, in certain embodiments, the cotton cells are derived and/or obtained, in whole or in part, from at least one cotton plant of a variety selected from PD 2164, SOUTHLAND M1, ACALA 5, CD3HCABCUH-1-89, FJA, TASHKENT 1, WESTERN STORMPROOF, PAYMASTER HS200, Pima S-7, and Acala MAXXA, or a progeny of any thereof. Certain varieties produced excellent growth. Thus, in certain embodiments, the cotton cells are derived and/or obtained, in whole or in part, from at least one cotton plant of a variety selected from PD 2164, ACALA 5, SOUTHLAND M1, CD3HCABCUH-1-89, FJA, Pima S-7, and Acala MAXXA, or a progeny of any thereof.

As shown in Table C, certain varieties produced good or excellent growth using cells obtained from an ovule, which may include ovule epidermal cells, located on the upper/top third of a boll, e.g., distal from the location on the boll to which it connects or connected to the stem of a cotton plant. Thus, in certain embodiments, the cotton cells are derived and/or obtained, in whole or in part, from ovule cells and/or ovule epidermal cells obtained from the top third of a cotton boll from at least one cotton plant of a variety selected from PD 2164, SOUTHLAND M1, ACALA 5, and CD3HCABCUH-1-89, or a progeny of any thereof. As shown in Table C, certain varieties produced excellent growth using ovule cells and/or ovule epidermal cells obtained from the top third of a cotton boll. Thus, in certain embodiments, the cotton cells are derived and/or obtained, in whole or in part, from ovule and/or ovule epidermal cells obtained from the top third of a cotton boll from at least on cotton plant of a variety selected from PD 2164 and ACALA 5, or a progeny of any thereof.

As shown in Table D, certain varieties produced good or excellent growth using ovule cells and/or ovule epidermal cells obtained from the middle third of the cotton boll. Thus, in certain embodiments, the cotton cells are derived and/or obtained, in whole or in part, from ovule cells and/or ovule epidermal cells obtained from the middle third of a cotton boll from a cotton plant of a variety selected from at least one cotton plant of a variety selected from PD 2164, SOUTHLAND M1, CD3HCABCUH-1-89, FJA, or a progeny of any thereof. As shown in Table D, certain varieties produced excellent growth using ovule cells and/or ovule epidermal cells obtained from the middle third of a cotton boll. Thus, in certain embodiments, the cotton cells are derived and/or obtained, in whole or in part, from ovule cells and/or ovule epidermal cells obtained from the middle third of a cotton boll from at least on cotton plant of a variety selected from PD 2164 and FJA, or a progeny of any thereof.

As shown in Table E, certain varieties produced good or excellent growth using ovule cells and/or ovule epidermal cells obtained from the bottom third of the boll. Thus, in certain embodiments, the cotton cells are derived and/or obtained, in whole or in part, from ovule cells and/or ovule epidermal cells obtained from the bottom third of a cotton boll from a cotton plant of a variety selected from at least one cotton plant of a variety selected from PD 2164, SOUTHLAND M1, TASHKENT 1, WESTERN STORMPROOF, ACALA 5, CD3HCABCUH-1-89, FJA, Pima S-7, Acala MAXXA, or a progeny of any thereof. As shown in Table E, certain varieties produced excellent growth using ovule cells and/or ovule epidermal cells obtained from the bottom third of a cotton boll. Thus, in certain embodiments, the cotton cells are derived and/or obtained, in whole or in part, from ovule cells and/or ovule epidermal cells obtained from the bottom third of a cotton boll from at least on cotton plant of a variety selected from PD 2164, SOUTHLAND M1, ACALA 5, CD3HCABCUH-1-89, FJA, Pima S-7, and Acala MAXXA, or a progeny of any thereof.

As shown in Tables C, D, and E, certain varieties produced good or excellent growth using cells from more than one ovule location. Thus, in certain embodiments, the cotton cells are derived and/or obtained, in whole or in part, from ovule cells and/or ovule epidermal cells obtained from at least one cotton plant of a variety selected from PD 2164, SOUTHLAND M1, ACALA 5, FJA, or a progeny of any thereof.

As shown in Tables C, D, and E, certain varieties quickly produced detectable levels of fiber in the grown cells. Accordingly, in certain embodiments, the cotton cells are derived and/or obtained, in whole or in part, from at least one cotton plant of a variety selected from SEALAND #1 (G.B.×G.H.), ACALA 5, SA 2413, TOOLE, M.U.8B UA 7-44, DIXIE KING, or a progeny of any thereof. As shown in Tables C, D, and E, certain varieties quickly produced detectable levels of fiber in the grown cells. As shown in Tables C and E, ACALA 5 showed both quickly detectable levels of fiber and excellent growth.

Thus, as the foregoing examples reveal, the presently disclosed in vitro methods of cotton production are amenable to using cotton cells with varying traits and genetic backgrounds. Accordingly, these methods can find clear use using cotton cells that express selected genes of interest.

### Example 10: Causing Differently Expressed Genes in Cotton Cells

The present invention includes the transformation and selection of transformed cells, and induction of those cells to elongate to form fiber.

In an exemplary assay cotton cells are obtained from the cotton varietals listed in Table F in accordance with the methods described herein. However, cotton cells obtained from any cotton varietal, including those specifically listed herein (such as those listed in Tables B-E), can be used in accordance with the methods of the invention.

TABLE F
Cotton varietals

TM-1
DP-50
DP-51
Coker 315
DP-90

The obtained cells are from cotton plants of the varietals in Table F and/or are the progeny of cells obtained from plants of these varietals. The cotton cells are obtained or are the progeny of cotton cells obtained from cotton plant or explant apical meristems, cotyledons, young leaves, hypocotyls, ovules, stems, mature leaves, flower, flower stalks, floral whorls, roots, bulbs, germinated seeds, somatic and zygotic embryo, and/or cambial meristematic cells (CMC). The obtained cells are isolated and proliferated with or without a callus phase.

The cells derived from suspension cultures or calli are then subject to a transformation using either an Agrobacterium or biolistic transformation. The transformation method used is either an Agrobacterium or biolistic transformation method based on prior published studies of cotton transformation to regenerate whole plants, however, in this case, cotton cells are transformed and elongated to form fiber rather than whole plants (Jin, 2005; Leelavathi, 2004; Finer, 1990).

In methods using Agrobacterium-mediated transformation, the cells are co-cultivated with an Agrobacterium carrying the genes of interest as well as the selection gene(s). The genes of interest include, for example, developmental regulatory genes and/or genes that modulate and/or improve cotton fiber development in vitro. In certain aspects, the gene(s) of interest or transformed with a promoter, under which expression of the gene(s) are controlled. The promoters include, for example, promoters that show preferential tissue and/or temporal expression in cotton plants or cotton cells. In methods using biolistic transformation, the cells are subject to bombardment with the genes of interest and the selection gene(s). After transformation, the cells are grown with media containing hormones to induce cell growth, a selection agent to inhibit the growth of untransformed cells due to the selection gene(s). For methods that employ Agrobacterium-mediated transformation, the media also includes antibiotic(s) to inhibit the growth of excess Agrobacterium. Cells are cultured and induced to elongate to fiber which is produced and harvested from the cultured cells.

Various publications are referenced throughout this application. Full citations for select references may be found listed at the end of the specification and preceding the claims. The disclosures of all referenced publication are hereby incorporated by reference in their entirety.

### REFERENCES

Bouchabké-Coussa, O., Obellianne, M., Linderme, D., Montes, E., Maia-Grondard, A., Vilaine, F., & Pannetier, C. (2013). Wuschel overexpression promotes somatic embryogenesis and induces organogenesis in cotton (Gossypium hirsutum L.) tissues cultured in vitro. Plant cell reports, 32 (5), 675-686.
Cai, C., Guo, W. & Zhang, B (2018). Genome-wide identification and characterization of SPL transcription factor family and their evolution and expression profiling analysis in cotton. Sci Rep 8, 762.
Deng F, Tu L, Tan J, Li Y, Nie Y, Zhang X (2012). GbPDF1 is involved in cotton fiber initiation via the core cis-element HDZIP2ATATHB2. Plant Physiology February 2012, 158 (2) 890-904.
Finer J J & McMullen M D (1990) Transformation of cotton (Gossypium hirsutum L.) via particle bombardment. Plant Cell Rep. 8:586-589.
Gordon-Kamm, B., Sardesai, N., Arling, M., Lowe, K., Hoerster, G., Betts, S., & Jones, A. T. (2019). Using Morphogenic Genes to Improve Recovery and Regeneration of Transgenic Plants. Plants (Basel, Switzerland), 8 (2), 38.
Hoerster, G., Wang, N., Ryan, L. et al. Use of non-integrating Zm-Wus2 vectors to enhance maize transformation. In Vitro Cell.Dev.Biol.-Plant 56, 265-279 (2020).
Jin et al., Molecular and Functional Dissection of the Maize B Chromosome Centromere, The Plant Cell May 2005, 17 (5) 1412-1423.
Khalilian, A., Marshall, M., Williams, P., Greene, J. and Porter, P. (2018) Cotton Production Systems for Soil and Energy Conservation in Coastal Plain Soils. American Journal of Plant Sciences, 9, 1500-1513.
Leelavathi et al., A simple and rapid Agrobacterium-mediated transformation protocol for cotton (Gossypium hirsutum L.): embryogenic calli as a source to generate large numbers of transgenic plants. Plant Cell Rep. 2004 February; 22(7):465-70.
Liu, Z.-H., Chen, Y., Wang, N.-N., Chen, Y.-H., Wei, N., Lu, R., Li, Y. and Li, X.-B. (2020). A basic helix-loop-helix protein (GhFP1) promotes fibre elongation of cotton (Gossypium hirsutum) by modulating brassinosteroid biosynthesis and signalling. New Phytol, 225:2439-2452.
Lowe, K., Wu, E., Wang, N., Hoerster, G., Hastings, C., Cho, M. J., Scelonge, C., Lenderts, B., Chamberlin, M., Cushatt, J., Wang, L., Ryan, L., Khan, T., Chow-Yiu, J., Hua, W., Yu, M., Banh, J., Bao, Z., Brink, K., Igo, E., . . . Gordon-Kamm, W. (2016). Morphogenic
Regulators Baby boom and Wuschel Improve Monocot Transformation. The Plant cell, 28 (9), 1998-2015.
Lowe, K., La Rota, M., Hoerster, G., Hastings, C., Wang, N., Chamberlin, M., Wu, E., Jones, T., & Gordon-Kamm, W. (2018). Rapid genotype “independent” Zea mays L. (maize) transformation via direct somatic embryogenesis. In vitro cellular & developmental biology. Plant: journal of the Tissue Culture Association, 54(3), 240-252.
Luo, K., Zhang, G., Deng, W. et al. (2008). Functional characterization of a cotton late embryogenesis-abundant D113 gene promoter in transgenic tobacco. Plant Cell Rep 27, 707-717.
Pu, L., Li, Q., Fan, X., Yang, W., Xue, Y. (2008). The R2R3 MYB transcription factor GhMYB109 is required for cotton fiber development. Genetics 180(2):811-820 (2008).
Shan, C., Shangguan, X., Zhao, B. et al. (2014). Control of cotton fibre elongation by a homeodomain transcription factor GhHOX3. Nat Commun 5, 5519.
Walford, S. A., Wu, Y., Llewellyn, D. J., & Dennis, E. S. (2012). Epidermal cell differentiation in cotton mediated by the homeodomain leucine zipper gene, GhHD-1. The Plant Journal: for cell and molecular biology, 71(3), 464-478.
Wang, S., Wang, J. W., Yu, N., Li, C. H., Luo, B., Gou, J. Y., Wang, L. J., & Chen, X. Y. (2004). Control of plant trichome development by a cotton fiber MYB gene. The Plant cell, 16(9), 2323-2334.
Zhang, M., Zheng, X., Song, S., Zeng, Q., Hou, L., Li, D., Zhao, J., Wei, Y., Li, X., Luo, M., Xiao, Y., Luo, X., Zhang, J., Xiang, C., & Pei, Y. (2011). Spatiotemporal manipulation of auxin biosynthesis in cotton ovule epidermal cells enhances fiber yield and quality. Nature biotechnology, 29(5), 453-458.
Zeng, YD., Sun, J L., Bu, SH. et al. EcoTILLING revealed SNPs in GhSus genes that are associated with fiber- and seed-related traits in upland cotton. Sci Rep 6, 29250 (2016).
Zheng, W., Zhang, X., Yang, Z., Wu, J., Li, F., Duan, L., Liu, C., Lu, L., Zhang, C., & Li, F. (2014). AtWuschel promotes formation of the embryogenic callus in Gossypium hirsutum. PloS one, 9(1), e87502.
Wan, P., Xu, D., Cong, S., Jiang, Y., Huang, Y., Wang, J., Wu, H., Wang, L., Wu, K., Carrière, Y., Mathias, A., Li, X., Bruce E. Tabashnik, B. (2017). Hybridizing transgenic Bt cotton with non-Bt cotton counters resistance in pink bollworm. PNAS, 114 (21) 5413-5418.

1. A method for producing cotton fiber, the method comprising:
modifying cotton cells such that they comprise at least one differently expressed gene, wherein said differently expressed gene affects growth of the modified cotton cells and/or improves and/or modulates cotton fiber development;
culturing the modified cotton cells in vitro; and
harvesting cotton fiber from the cultured cells.

2. The method of claim 1, wherein the cotton fiber is harvested directly from the cultured cells.

3. The method of claim 2, wherein the cotton fiber is harvested from the cells without growing a cotton plant.

4. The method of claim 3, wherein the differently expressed gene is a gene selected for improvement and/or modulation of cotton fiber development or a developmental regulatory gene.

5. The method of claim 4, wherein the differently expressed gene is a transgene.

6. The method of claim 5, wherein the transgene is operably connected to a promoter.

7. The method of claim 6, wherein the promotor is selected from a D113 promoter, a GbPDF1 promoter, a GhMYB109 promoter, a Stunt7 (S7) promoter, a floral binding protein 7 (FBP7) promoter, a TA29 promoter, and a CaMV 35S promoter, a cotton fiber-specific GhSCFP promoter, an actin promoter, such a GhACT1 promoter, a petunia MADS box gene floral binding protein 7 (FBP7) promoter.

8. The method of claim 6, wherein modifying comprises introducing the transgene and promoter via transformation.

9. The method of claim 6, wherein the differently expressed gene is expressed at a higher level compared with a cotton cell prior to the modifying.

10. The method of claim 9, wherein the differently expressed gene is a developmental regulatory gene.

11. The method of claim 9, wherein the differently expressed gene decreases doubling time of the cells during the culturing.

12. The method of claim 10, wherein the gene is selected from maize WUSCHEL gene Wuschel2 (Wus2); an Arabidopsis WUSCHEL gene (AtWUS); a cotton WUSCHEL gene (GhWus); a maize BABY BOOM gene (Bbm).

13. The method of claim 9, wherein the differently expressed gene is a gene selected for improvement and/or modulation of cotton fiber development.

14. The method of claim 13, wherein the differently expressed gene increases cotton fiber yield, increases cotton fiber length, and/or increases cotton fiber strength.

15. The method of claim 13, wherein the differently expressed gene is selected from a homeodomain-leucine zipper transcription factor GhHOX3 gene; a putative homeodomain leucine zipper transcription factor GhHD-1 gene; an SBP gene family of transcription factors gene; a SQUASOMA promoter-binding protein-like transcription factor 5 (SPL5) gene; a GL1-like MYB protein encoding gene GaMYB2; a GhACT gene, a GhACT1 gene, an IAA biosynthetic gene iaaM; an auxin efflux protein gene GhPIN3a; a cotton LIM protein gene GhWlim5; a basic helix-loop-helix protein encoding gene GhFP1; a cotton sucrose synthase gene (GhSus); a cotton PROTODERMAL FACTOR1 (GbPDF1) gene; a cotton fiber specific RDR3 MYB gene GhMYB109; and a sucrose-synthesis related gene.

16. The method of claim 1, wherein prior to modifying, the cotton cells are obtained or are the progeny of cotton cells obtained from at least one cotton plant.

17. The method of claim 16, wherein the cotton cells are obtained or are the progeny of cotton cells obtained from cotton plant or explant apical meristems, cotyledons, young leaves, hypocotyls, ovules, stems, mature leaves, flower, flower stalks, floral whorls, roots, bulbs, germinated seeds, somatic and zygotic embryo, and/or cambial meristematic cells (CMC).

18. The method of claim 1, wherein the modified cotton cells are not transgenic.

19. The method of claim 18, wherein the differently expressed gene is endogenous to the modified cotton cells.

20. The method of claim 19, wherein modifying comprises contacting the cotton cell with at least one mutagen, wherein the mutagen comprises an endonuclease, a chemical mutagen, and/or radiation.


SEQUENCE LISTING (69 sequences)

SEQ ID NO: 1 - DNA, 2136 bp, Gossypium hirsutum
atggattgcggaagcggcggcgtaggagctagtggttccggcggagaccatgattcctccgacctttcacgacggaaaaagccttaccatcgtcacactgctcaccagattcagaggcttgaatcgatgttcaaggaatgtccacacccagacgagaaacaaaggttacatctaagtagggaattgggattagcaccaagacagatcaagttttggtttcaaaataggaggacacaaatgaaggcacaacatgaaagagccgataactctgcacttcgtgctgagaacgataagatccgatgtgaaaacatagccataagagaagcacttaagaatgtgatttgtccttcttgtggtggtcctcctgttactgaagattcttattttgatgaccagaaaatgagaatggagaatgctcaattaaaggaagagcttgatagagtatccagcattgcagccaagtacatagggagaccaatatcccaactcccaccggtacaacctgttcatatctcttcattagatatcaggatggcgagttttgacggctatggtgtcggcgctggtccttcactcgatctcgatctcctccctggaagttcgtcatcaatgccgaatttaccgttccaaccggtcgttatttcagacatggacaagtccctcatgtctgatatagccgcaaatgcaatggaagaactgcttaggctattacaaaccaatgaaccattgtggattaaatccactaatgatggcaaagatgttcttaatctcgaaagttatgaaagaatcttccctaagcctaataatactcattttaaatctcctaatattagggtcgaagcttctcgagattccggtgttgttattatgaatggcttagctttggtcgacatgttcatggattccaacaaatgggtggagctatttcccaccattgtgtcaattgccaaaacaatcgaagtgatttcacctgggatgttgggtacacatagtggttctttgcaattgatgtatgaagaactacaggtcttgtctccattagtaccaacacgagaattttatacactccgatattgtcaacaaattgaacaagggttatgggctattgtcaatgtttcatatgatttgccacaatttgcttctcaatgtcgatctcatagactcccttctggttgtttgattcaagacatgcctaatggttactccaaggttacttggttagaacgtgtggagattgaagacaaaaccccgattcatcgattgtatcgagaccttgttcatagtggttcagcatttggagccgaacggtggctcacgacccttcaaaggatgtgcgaatggtttgcttgcttaagggtatcgagcacttcgactcgggatctcggtggagtgattccatctcccgagggcaggagaagcatgatgaagctagctcaaaggatggtaaacaatttctgcacgagcgtcggcacgtcgaatagtcatcggtcgacaacactttccggttcgaatgaagttggtgttcgggtcaccgttcataagagttctgatcccggacaacccaacggtatagttcttagtgcagctactacattttggctccctgttagtccacaaaatgtcttcaatttcttcaaagatgaaagaactagaccacagtgggatgttctatccagcggcaacgcagtccaagaagttgctcatatcgcaaacggatcacatcccggcaactgtatatccgttttacgagcctttaatacaagtcacaacaacatgttaatactacaagagagttgcatagacagttctggttcactggtggtatactgtccagttgatttaccagctattaatgtagcaatgagcggcgaagatccttcttacattccgttacttccgtcaggttttacgataacacccgacggtcatctcgagcaaggtgacggagcatcgacgagttctagtaccgggcacggaagatcgtccggtggttcattgattacggtagcgtttcaaatactagtgagcagcttgccatcggcaaaattaaacttggattcggtcacaactgttaataaccttattggtactactgtacaacagattaaggctgctttgaattgtcctagttcttga

SEQ ID NO: 2 - protein, 711 aa, Gossypium hirsutum
MDCGSGGVGASGSGGDHDSSDLSRRKKPYHRHTAHQIQRLESMFKECPHPDEKQRLHLSRELGLAPRQIKFWFQNRRTQMKAQHERADNSALRAENDKIRCENIAIREALKNVICPSCGGPPVTEDSYFDDQKMRMENAQLKEELDRVSSIAAKYIGRPISQLPPVQPVHISSLDIRMASFDGYGVGAGPSLDLDLLPGSSSSMPNLPFQPVVISDMDKSLMSDIAANAMEELLRLLQTNEPLWIKSTNDGKDVLNLESYERIFPKPNNTHFKSPNIRVEASRDSGVVIMNGLALVDMFMDSNKWVELFPTIVSIAKTIEVISPGMLGTHSGSLQLMYEELQVLSPLVPTREFYTLRYCQQIEQGLWAIVNVSYDLPQFASQCRSHRLPSGCLIQDMPNGYSKVTWLERVEIEDKTPIHRLYRDLVHSGSAFGAERWLTTLQRMCEWFACLRVSSTSTRDLGGVIPSPEGRRSMMKLAQRMVNNFCTSVGTSNSHRSTTLSGSNEVGVRVTVHKSSDPGQPNGIVLSAATTFWLPVSPQNVFNFFKDERTRPQWDVLSSGNAVQEVAHIANGSHPGNCISVLRAFNTSHNNMLILQESCIDSSGSLVVYCPVDLPAINVAMSGEDPSYIPLLPSGFTITPDGHLEQGDGASTSSSTGHGRSSGGSLITVAFQILVSSLPSAKLNLDSVTTVNNLIGTTVQQIKAALNCPSS

SEQ ID NO: 3 - DNA, 4631 bp, Gossypium hirsutum
ttccatcactcgcaattacacctctcacccacaataaatgctattgcaccctctcgatgggctctgctattaactccttagctgccacatcctctccacctgtttcatcaattctcttcattttcctcacccccccttggaacttcaaacccctttctctctctctagactttaaccctcttctctctcaaggtaggtctctgtcaaaccagtgaccccaaacttttaaaaaccaccctaaaacctcctttcattaatctttctcaacaatgttcaaaagggctttcttttgttttgcctatacggccctgtaaaaagtctctactcacgaaacctatttctttccctggaccagcttgtttatagcacccgatctgtgttcacaccatttgccattctctctttcatatattcgcttataaagctttgctgagaatcaagagagagtgcaaaaagtgtttgtaatctttggtttgtggggactttctatcaaaatcaacctaaataccaccaatataggaaagcggggccaagaatctaacaagcaagagactgatatgtgtagcatgtattgctgctggttagggttttaaaggtcaatggctatctatgtctcccaacatgaggtttctagtgaccaacttaccactgcatttaagacaagaaaatagttgccctaagctctaagtccccttctccaaataatccataaggtaattttctttcctcctttttttccccaacttttgctgcccattttatttctccgaggttctttcacttttctcatgggaatagattaggataagctttcgactaatccatcattttaatcttcgacgtatatgctacctttaaatatagtagcagttggttattaagatattcaaagaaaaatccttctggataagcgaactagaccttttttttctttgaaagagagatggtatatggtttacagctccattgtttatttgcctttcaaatgttttacgattccattgtcatgcactggttttgacttttttaactttgtacttacattttgtgtctttcgttactgttctacagtttatttaacccttttccccctttattttagtttgcttgacttgtgtatgttctgtcggcagaacccagaatatccataccgtcgacattcgagcactttgttaacatgtttagccccaacttatttgagagtccccatatgttcgacatgtctcataagacctcggaaagtgaactaatggggaagatcagggatgatgattatgagatcaaatcagtcaatgaaactatggatgctccctctggagatgatcaagatcctgaccaacgccctaaaaagaagcgttatcatcgtcatacccagcgtcaaatccaggagatggaagcgtaagtcttgtttttttttatcctcttacttaaagataagaaagattaacatgactagttcttctttggggaattgattctttggacactttgttgctcagattctttaaggaatgccctcatcctgatgataagcaaaggaaggagcttggccgtgagctagggttagaacctctacaagtcaagttttggttccaaaacaagcgcacccaaatgaaggtatatttccatgtataggtgaaaatcaggagctctaatatgcttatttaagtatttagactcgttttatgagtaagcttttctgcctatgtagtaagctttcctagctttgttgctaaagttcaccttcattaacaggcccaacatgaacgccatgaaaatgctatactgaaggctgagaatgaaaaactccgagctgagaataataggtacaaggaagctctcagcaatgctacatgccccagctgtggaggcccagctgcccttggagagatgtcatttgatgagcaacatttgagaatagaaaatgctcggttaagggaagaggtgattgaaaaatgcttcacacatcccatcttattatcaagttacctttttattatactttttatattttggttttcttgtattacagattgataggatatctggaatagctgctaaatatgttggcaagcctttatcttcattgcctcacctttcatctcatttacattcgcgctctgctgatcttggagctagcaatttcgggaatcaatcaggatttgtaggggaaatggatcgcagtggtgatcttctgaggtctgtctctggacctacagaagcggataagcccatgattgttgagcttgctgttgctgcaatggaggaactaatacgaatggcccaatctggggaacctttgtgggttcctggggacaattctacagatgtgttgaacgaagatgaatacttaagaactttccctaggggaattggaccaaagcctttggggttgaggtctgaagcttcaagagaatctgcagttgtcatcatgaatcatgtcaacttagttgagattctcatggatgtggtaggattttcttttccccaagtttgggtccattatataaatttttagatccacctccacttatttatgacaaaaacgattgatttccagaatcaatggtcaagtgtgttttgcggtattgtttcaagggctatgactttagaagtcctatcaactggagttgcaggaaaatgcaatggggccttgcaagtggtactaagtacatctatatgcttttcttttcaatagcaatctaataaaattttctgccatgcatgctctttccctaagctctgaaagcatgcctaacatcttgcattttcagatgacggctgagttccaagtcccttcaccacttgtaccaactcgggaaaattatttcgcgaggtactgtaagcagcatattgatggaacttgggcagtggttgatgtttccttggataatttacgccctaacccaatgtccaagtgtagaagaaggccctcaggttgcttgatccaagaattgccaaatggatactctaaggtaacttactctttatttcaacgtgtatatatttgttgggctgcattttatgttgccgcttaacgtgtacacaatgtcatgttttcattatactttcaggttatatgggtcgagcatgtagaagtggatgatagagctgtccacaacatatacagaccagtagttaattccggtctagcttttggagcaaaacgttgggtggctacgttggatcgacagtgtgagcgtctagcaagttcaatggccagtaacattccagcaggggatctatgcggtaacaccatgtttagcagcttataattctcttagtcctagggataccttgttgaatgcagcgcattccatgtttctctgatgttaccatatcggttccttttgatctctgcagttataacaagcccagaagggaggaaaagtatgttgaagttggcagagaggatggtgactagcttttgtacaggtgttggtgcttctacggcccatgcttggacaactttatcggcaacaggctccgatgatgtgcgggttatgacccgaaagagcatggatgatccaggaaggcctcctggtattgtacttagtgctgcaacttccttctggatccaagttccaccaaagagggtatttgatttcctaagggatgagaactctagaagtgaggtacattaattaagcttccaattatgccaaaacaatggtacactttttatgttagaatattatactaatttgcagtttttaaattgaatttatagtgggatatcctatcaaatggtggcctagttcaagaaatggctcacatagctaatggtcgtgatccaggcaattgtgtctctttactccgcgtaaatgtaagttggcagttaacacatgctttagatctttaattgacgacaatcatccatcaagctaacattctaactggaaaccgatctgcagagtgcaaactctagccaaagcaacatgttgatacttcaagagagctgcactgatgctaaagggtcctacgtgatatatgccccggtcaatattgttgcaatgaacatcgtcttaagtggcggggacccggattatgtcgcactattgccatccggtttcgcaattcttcccgatggtccaggagttaatggaggagggattctcgaaatcggctcgggtggctctctccttaccgttgctttccagattttggttgattcagttcccacagcaaagctttctcttggatcagtggcgactgtcaacagtctaattaaatgcacggttgaaaggatcaaggctgccgtaaagtgcaataatgcttgaccaaacatgatataaaaaaaggtgagcaatcaacttaattaatccttgactatatttggagtttgcaatctaagcttatcccatttcacaatgtgctgcaaggaaacgagaagaaaaggtgtttgtccgaaaacaaatttaacgattgaagaagtcaagagcgcacctttcaattcatcctttgcggtcatggtgttctgtaagaaggcaaaatcatcaagcctgcaaggatagtaggttcgggaattgactttgccaacgagattctaatattagatatgttgggagaactccccattttgtgtaggctaagagttcaatgtaggagtggactttatactagtctaattt

SEQ ID NO: 4 - protein, 725 aa, Gossypium hirsutum
MFSPNLFESPHMFDMSHKTSESELMGKIRDDDYEIKSVNETMDAPSGDDQDPDQRPKKKRYHRHTQRQIQEMEAFFKECPHPDDKQRKELGRELGLEPLQVKFWFQNKRTQMKAQHERHENAILKAENEKLRAENNRYKEALSNATCPSCGGPAALGEMSFDEQHLRIENARLREEIDRISGIAAKYVGKPLSSLPHLSSHLHSRSADLGASNFGNQSGFVGEMDRSGDLLRSVSGPTEADKPMIVELAVAAMEELIRMAQSGEPLWVPGDNSTDVLNEDEYLRTFPRGIGPKPLGLRSEASRESAVVIMNHVNLVEILMDVNQWSSVFCGIVSRAMTLEVLSTGVAGKCNGALQVMTAEFQVPSPLVPTRENYFARYCKQHIDGTWAVVDVSLDNLRPNPMSKCRRRPSGCLIQELPNGYSKVIWVEHVEVDDRAVHNIYRPVVNSGLAFGAKRWVATLDRQCERLASSMASNIPAGDLCVITSPEGRKSMLKLAERMVTSFCTGVGASTAHAWTTLSATGSDDVRVMTRKSMDDPGRPPGIVLSAATSFWIQVPPKRVFDFLRDENSRSEWDILSNGGLVQEMAHIANGRDPGNCVSLLRVNSANSSQSNMLILQESCTDAKGSYVIYAPVNIVAMNIVLSGGDPDYVALLPSGFAILPDGPGVNGGGILEIGSGGSLLTVAFQILVDSVPTAKLSLGSVATVNSLIKCTVERIKAAVKCNNA

SEQ ID NO: 5 - DNA, 2178 bp, Gossypium hirsutum
atgtttagccccaacttatttgagagtccccatatgttcgacatgtctcataagacctcggaaagtgaactaatggggaagatcagggatgatgattatgagatcaaatcagtcaatgaaactatggatgctccctctggagatgatcaagatcctgaccaacgccctaaaaagaagcgttatcatcgtcatacccagcgtcaaatccaggagatggaagcattctttaaggaatgccctcatcctgatgataagcaaaggaaggagcttggccgtgagctagggttagaacctctacaagtcaagttttggttccaaaacaagcgcacccaaatgaaggcccaacatgaacgccatgaaaatgctatactgaaggctgagaatgaaaaactccgagctgagaataataggtacaaggaagctctcagcaatgctacatgccccagctgtggaggcccagctgcccttggagagatgtcatttgatgagcaacatttgagaatagaaaatgctcggttaagggaagagattgataggatatctggaatagctgctaaatatgttggcaagcctttatcttcattgcctcacctttcatctcatttacattcgcgctctgctgatcttggagctagcaatttcgggaatcaatcaggatttgtaggggaaatggatcgcagtggtgatcttctgaggtctgtctctggacctacagaagcggataagcccatgattgttgagcttgctgttgctgcaatggaggaactaatacgaatggcccaatctggggaacctttgtgggttcctggggacaattctacagatgtgttgaacgaagatgaatacttaagaactttccctaggggaattggaccaaagcctttggggttgaggtctgaagcttcaagagaatctgcagttgtcatcatgaatcatgtcaacttagttgagattctcatggatgtgaatcaatggtcaagtgtgttttgcggtattgtttcaagggctatgactttagaagtcctatcaactggagttgcaggaaaatgcaatggggccttgcaagtgatgacggctgagttccaagtcccttcaccacttgtaccaactcgggaaaattatttcgcgaggtactgtaagcagcatattgatggaacttgggcagtggttgatgtttccttggataatttacgccctaacccaatgtccaagtgtagaagaaggccctcaggttgcttgatccaagaattgccaaatggatactctaaggttatatgggtcgagcatgtagaagtggatgatagagctgtccacaacatatacagaccagtagttaattccggtctagcttttggagcaaaacgttgggtggctacgttggatcgacagtgtgagcgtctagcaagttcaatggccagtaacattccagcaggggatctatgcgttataacaagcccagaagggaggaaaagtatgttgaagttggcagagaggatggtgactagcttttgtacaggtgttggtgcttctacggcccatgcttggacaactttatcggcaacaggctccgatgatgtgcgggttatgacccgaaagagcatggatgatccaggaaggcctcctggtattgtacttagtgctgcaacttccttctggatccaagttccaccaaagagggtatttgatttcctaagggatgagaactctagaagtgagtgggatatcctatcaaatggtggcctagttcaagaaatggctcacatagctaatggtcgtgatccaggcaattgtgtctctttactccgcgtaaatagtgcaaactctagccaaagcaacatgttgatacttcaagagagctgcactgatgctaaagggtcctacgtgatatatgccccggtcaatattgttgcaatgaacatcgtcttaagtggcggggacccggattatgtcgcactattgccatccggtttcgcaattcttcccgatggtccaggagttaatggaggagggattctcgaaatcggctcgggtggctctctccttaccgttgctttccagattttggttgattcagttcccacagcaaagctttctcttggatcagtggcgactgtcaacagtctaattaaatgcacggttgaaaggatcaaggctgccgtaaagtgcaataatgcttga

SEQ ID NO: 6 - DNA, 6003 bp, Gossypium hirsutum
tgcaataaaagtaaataaatacataaacagatcacagatgagttgaattaatcatataccgaccggatatgattatatgtatgcatatatgtcaaaaggaagagacgagattaaatttggttagcaattatgagaattgcaccacatagattttggtaggcatgcatgcatggacatgatatataatgcgtctgaaatctgcaaatgcagatcatagcctgtatccttcagactcaataatttcttggaagatttgagagcaagccgaaagattaaatatatttcagttattctagcagcagctctaatttgcttttcatgtaatttgatggggaaattaattagtataatattcaaccatatataaagcaaatatttaattccaaaacaaaacaataaatatttgaaagtagtcgttatgatttaaacttgtgagatatcaattatatcttttatcttatgaaaaatttaaactttttatactatatgtgtacacagtgaatattgttgcagatttcatgacaatattaggaagaaatatttctcttgatttgagaatttatgatgttgctccaagttgattgagatgaaaattttacaagacagtattgatattagatataagtcctgttattagtaaatatatttaacttcttttatctacaaatatatatatatcctatacctgcatgcttattagtaaaacctatcaatattggagctgagacactgaaaactagttgaaagcttacaaagttaatttaaagaaagggtagggaaggggagatgtttttctctttctataatttttatttctatcactcgcaattacacctctcacccaccataaatgctattgcaccctctcgatgggctctgctattaactccttagctgccacatcctctccacctgtttcttcaattctcttcattttcctcaaccccccctaggaacttcaaacccctttctctctctagactttaaccctattctctctcaaggtaggtctgtcaaaccagtgaccccaaactcttaaaaaccaccctaaagcctcctttcattaatctttctcaacaatgttcaaaagggttttcttttgttttgcctatacggccctttaaaaagtctctactcacaaaacctatttctttccctggaccagcttgtttatagcacccgatctgtgttcacaacatttgccattctctctttcataaattcgcttataaagctttgctgagaatcaagagagagtgcaaaaagtgtttgtaatctttggtttgtggggactttctatcaaaatcaacctaaataccaccaatataggaaaccggggccaagaatctaacaagcaagagactgatatgtgtagcatgtattgctgctggttagggttttaaaggtcatcggctatctatgtctcccaacatgagatttctagtgaccaacttaccactgcatttaagacaagaaaatagttgccctaagctcttagtctcctcctccaaataatccatatataaggtaatttctttcctcctttttttccccaacttttgctgctctttttatttctccgaggttctttcacttttcccatgggaatagattacgataagctttcgactaatccatcattttaatcttcgaagtatatgctacctttaaatatagtagcagttggttattaagatattcaaagaaaaatccttctcgataagcgaactaaaactttttttctttgaaagagagatggtatatggtttacagctccactgtttatttgccttccaaatgtttcgacgattcgattgtcatgcactggttttgacttttttaaccttctttatgacttctttgtatttacattttgtgtctttcgttactgttctacagtttatttaacccttttccccctttattttagtttgcttgatttgtgtatgttctctcggcagaacccagaatatccataccgtcgacattcgagcactttgttaacatgtttagccccaacttatttgagagtccccatatgttcgacatgtctcataagacctcggaaagtgaactaatggggaaggtcagggatgatgattatgagatcaaatcagtcactgaaactatggatgctccctctggagatgatcaagatcctgaccaacgccctaaaatgaagtgttatcatcgtcatacccagcgtcaaatccaggagatggaagcgtaagttttttttttcctcttacttaaagataagaaatattaacatgactagttcttctttggggaattgattgttttgacactttgttgctcagattctttaaggagtgccctcaccctgatgataagcaaaggaaggagcttggccgtgagctagggttagaacctcttcaagtcaagttttggttccaaaacaagcgcacccaaatgaaggtatatttccatatataggtgaaaatcaggagctctaatctgcttatttaagtatttagacttgttttatgattaagcttttctgtctatgtagtaagctttcctagcttagttgctaaagttcaccttcattaacaggcccaacatgaacgccatgaaaatgctatactgaaggctgagaatgaaaaactccgagccgagaataataggtacaaggaagctctcagcaatgctacatgccccagctgtggaggcccagctgcccttggagagatgtcatttgatgagcaacttttgagaatagaaaatgctcggttaagggaagaggtgattgaaaaatgcctcacacatcccatcttattatcaagttacctttttattgtactttttatattttggttttcttgtattacagattgataggatatctggaatagctgctaaatatgttggcaagcctttatcttctttgcctcacctttcatctcatttacattcgcgctctgttgatcttggagctagcaatttcgggacacaatcaggatttgtaggggaaatggatcgcagtggtgatcttctgaggtctgtctctggacctacagaagcggataagcccatgattgttgagcttgctgttgctgcaatggaggaactaatacgaatggcccaatctggggaacctttgtgggttcctggggacaattctatagatgtgttgagcgaagatgaatacttaagaactttccctaggggaattggaccaaagcctttggggttgaggtctgaagcttcaagagaatctgcagttgtcatcatgaatcatgtcaacttagttgagattctcatggatgtggtaggattttttttttccccaagtttgggtccattatataaatttttagatccacccccatttatttatgacaaaaacgattgatttccagaatcaatggtcaagtgtgttttgcggtattgtttcaagggctatgactttagaagtcctatcaactggagttgcaggaaactacaatggagccttgcaagtggtactaagtacatctatatgcttttcttttcaataccaatctaataaaactttctggcatgcatgctctttccctaagctctaaaagcatgcctaacatcttgcattttcagatgacggctgagttccaagtcccttcaccacttgtaccaactcgggaaaattatttcgtgaggtactgtaagcagcatattgatggaacttgggcagtggttgatgtttccttggataatttacgccctaacccaatgtccaagtgtagaagaaggccctcaggttgcttgatccaagaattgccaaatggatactctaaggtaacttactctttatttcaatgagtatatatttgttgggctgcattttatgttgccgcttaacgtgtatacaatgtcatgttttcattatactttcaggttatatgggtcgagcatgtagaagtggatgatagagctatccacaacatatacagaccagtagttaattccggtctagcttttggagcaaaacgttgggtggctacgttagatcgacagtgtgagcgtctagcaagttcaatggccagtaacattccggcaggggatctatgcggtaacaccatgtttagcagcttataattctcttagtcctagggataccttgttgaatgcagtgcattccatgtttctctgatgttaccatatcgcttccttttgatctctgcagttataacaagcctagaagggaggaaaagtatgttgaagttggcagagaggatggtgactagcttttgtacaggtgttggtgcttctacggcccatgcttggacaagtttatcggcaacaggctccgatgatgtgcgggttatgacccgaaagagcatggatgatccaggaaggcctcctggtattgtacttagtgctgcaacttccttctggatcccagttccaccaaagagggtatttgatttcctaagggatgagaactctagaagtgaggtacattaactaagcttccaataaagccaaaacaatggtacactttttatgttagaatattatactaatttgcagtttttaaattgaatttatagtgggatatcctatcaaatggtggcctagttcaagaaatggctcacatagctaatggtcgtgatccaggcaattgtgtctctttactgcgcgtaaatgtaagttggcaagttaaacacatgctttagatctttaattgacgacaatcatccattaagttaacatctctaactggaaaccgatctgcagagtgcaaactctagccaaagcaacatgttgatacttcaagagagctgcactgatgctacagggtcctatgtgatatatgccccggtcgatattgttgcaatgaacgtcgtcttaagtgggggggacccggattatctcgcactattgccatccggtttcgcaattctacccgatggtccaggagttaatggaggagggattctcgaaataggctcgggtggctctctccttaccgttgctttccagattttggttgattcagttccaacagcaaagctttcccttggatcagtgacgactgtcaatagtctaattaaatgcacagttgaaaggatcaaggctgccgtaatgtgcaataatgcttgaccaaagatgatataaaaaaaggtgagcaatcaacttaattaatccttcactatatttggagtttgcaatctaagcttatcccatttcacaatgtgctgcagggaaaagagaagaaaaggtgtttgtccgaaaacaaatttaacgattaaagaagtcaagagcgcacctttcaattcatcctttgcggtcatggtgttttgtaagaaggcaaaatcaccaagcctgcaaggatagtaggttcgggaattgactttgccaaagagattttaatattagatatgttgggagaactccccattttgtgtaggctaagagttcaatgtaggagtggactttatactagtctaatttcttttcggtttcatgtgttaatgttgaagcattagttattttggacttattcctccattaacaaacatttgttaatttctgctttattttttgttttggttgtttcttaaattaatgaatagcttagctgctgcctttctcctacttttcttatgcagaatcgcattttatttttagttggacatcactactgtagatatagcttattagggacactcccaacaacatcagcttatttccctctaagaccgatagatgtttaaggtacagtggagtgaacgtatcgctccataattaatatattttgtttcattaattgttgtatgaatgtgtaattttgaaaaccgatgttagatctagtctcaacttttcttttaatttttttttatcaaggtatccgttacgaatgtgcaatatttttattttaaagaagtatataatactcaattttatttataactatatatacaacacaaggtgcatacaccacgttagctcttttttttttattctttatcatctattagagttttttttttataaaaa

SEQ ID NO: 7 - DNA, 2178 bp, Gossypium hirsutum
atgtttagccccaacttatttgagagtccccatatgttcgacatgtctcataagacctcggaaagtgaactaatggggaaggtcagggatgatgattatgagatcaaatcagtcactgaaactatggatgctccctctggagatgatcaagatcctgaccaacgccctaaaatgaagtgttatcatcgtcatacccagcgtcaaatccaggagatggaagcattctttaaggagtgccctcaccctgatgataagcaaaggaaggagcttggccgtgagctagggttagaacctcttcaagtcaagttttggttccaaaacaagcgcacccaaatgaaggcccaacatgaacgccatgaaaatgctatactgaaggctgagaatgaaaaactccgagccgagaataataggtacaaggaagctctcagcaatgctacatgccccagctgtggaggcccagctgcccttggagagatgtcatttgatgagcaacttttgagaatagaaaatgctcggttaagggaagagattgataggatatctggaatagctgctaaatatgttggcaagcctttatcttctttgcctcacctttcatctcatttacattcgcgctctgttgatcttggagctagcaatttcgggacacaatcaggatttgtaggggaaatggatcgcagtggtgatcttctgaggtctgtctctggacctacagaagcggataagcccatgattgttgagcttgctgttgctgcaatggaggaactaatacgaatggcccaatctggggaacctttgtgggttcctggggacaattctatagatgtgttgagcgaagatgaatacttaagaactttccctaggggaattggaccaaagcctttggggttgaggtctgaagcttcaagagaatctgcagttgtcatcatgaatcatgtcaacttagttgagattctcatggatgtgaatcaatggtcaagtgtgttttgcggtattgtttcaagggctatgactttagaagtcctatcaactggagttgcaggaaactacaatggagccttgcaagtgatgacggctgagttccaagtcccttcaccacttgtaccaactcgggaaaattatttcgtgaggtactgtaagcagcatattgatggaacttgggcagtggttgatgtttccttggataatttacgccctaacccaatgtccaagtgtagaagaaggccctcaggttgcttgatccaagaattgccaaatggatactctaaggttatatgggtcgagcatgtagaagtggatgatagagctatccacaacatatacagaccagtagttaattccggtctagcttttggagcaaaacgttgggtggctacgttagatcgacagtgtgagcgtctagcaagttcaatggccagtaacattccggcaggggatctatgcgttataacaagcctagaagggaggaaaagtatgttgaagttggcagagaggatggtgactagcttttgtacaggtgttggtgcttctacggcccatgcttggacaagtttatcggcaacaggctccgatgatgtgcgggttatgacccgaaagagcatggatgatccaggaaggcctcctggtattgtacttagtgctgcaacttccttctggatcccagttccaccaaagagggtatttgatttcctaagggatgagaactctagaagtgagtgggatatcctatcaaatggtggcctagttcaagaaatggctcacatagctaatggtcgtgatccaggcaattgtgtctctttactgcgcgtaaatagtgcaaactctagccaaagcaacatgttgatacttcaagagagctgcactgatgctacagggtcctatgtgatatatgccccggtcgatattgttgcaatgaacgtcgtcttaagtgggggggacccggattatctcgcactattgccatccggtttcgcaattctacccgatggtccaggagttaatggaggagggattctcgaaataggctcgggtggctctctccttaccgttgctttccagattttggttgattcagttccaacagcaaagctttcccttggatcagtgacgactgtcaatagtctaattaaatgcacagttgaaaggatcaaggctgccgtaatgtgcaataatgcttga

SEQ ID NO: 8 - protein, 725 aa, Gossypium hirsutum
MFSPNLFESPHMFDMSHKTSESELMGKVRDDDYEIKSVTETMDAPSGDDQDPDQRPKMKCYHRHTQRQIQEMEAFFKECPHPDDKQRKELGRELGLEPLQVKFWFQNKRTQMKAQHERHENAILKAENEKLRAENNRYKEALSNATCPSCGGPAALGEMSFDEQLLRIENARLREEIDRISGIAAKYVGKPLSSLPHLSSHLHSRSVDLGASNFGTQSGFVGEMDRSGDLLRSVSGPTEADKPMIVELAVAAMEELIRMAQSGEPLWVPGDNSIDVLSEDEYLRTFPRGIGPKPLGLRSEASRESAVVIMNHVNLVEILMDVNQWSSVFCGIVSRAMTLEVLSTGVAGNYNGALQVMTAEFQVPSPLVPTRENYFVRYCKQHIDGTWAVVDVSLDNLRPNPMSKCRRRPSGCLIQELPNGYSKVIWVEHVEVDDRAIHNIYRPVVNSGLAFGAKRWVATLDRQCERLASSMASNIPAGDLCVITSLEGRKSMLKLAERMVTSFCTGVGASTAHAWTSLSATGSDDVRVMTRKSMDDPGRPPGIVLSAATSFWIPVPPKRVFDFLRDENSRSEWDILSNGGLVQEMAHIANGRDPGNCVSLLRVNSANSSQSNMLILQESCTDATGSYVIYAPVDIVAMNVVLSGGDPDYLALLPSGFAILPDGPGVNGGGILEIGSGGSLLTVAFQILVDSVPTAKLSLGSVTTVNSLIKCTVERIKAAVMCNNA

SEQ ID NO: 9 - DNA, 1637 bp, Gossypium hirsutum
cagtattgtaaaagatggccgacggtgaggctattcaacccctcgtctgtgataatggaactggaatggtgaaggccggttttgctggtgatgatgctccaagggcagtttttcccagtatcgttggtcgtccccggcacactggtgttatggttgggatgggtcagaaggatgcctatgtaggagatgaagcacaatctaaaggaggtatccttactttgaaatatcctattgagcatggtattgtgagcaattgggatgatatggaaaagatctggcatcatacattctacaacgaactccgtgttgttcctgaggagctccctgtgctactcacggaagcacctctcaaccccaaggccaatagagaaaagaagactcagatcatgtttgagaccttcaatgtacctgctatgtatgttgccatccaggccgttctctctctgtatgccagtggtcgtacaacaggtattgtgctggattccggtgatggtgtttctcacatcgtgccaatctatgaaggatatgcccttccacatgccatcctccgtcttgaccttgcaggtcgtgatctaaccgatgccttgatgaagattcttaccgagagaggttacatgttcaccaccactgctgaacgggaaattgtccgtcacatgaaagagaagcttgcttatgttgccctggactatgagcaggagctggagactgccaagagcagctcatctgttgagaagaactatgagttgcctgacggacaagttattactattgaagctgagagattccgttgcccggaagtcctcttccagccatctttcatcgggatggaagctgctggaatccatgaaactacctacaactctatcatgaagtgtgatgtggatatcaggaaggatctctacggtaacattgtgctcagtgggggttcaaccatgttccccggtattgcagaccgcatgagcaaggagatcactgcacttgctccaagcagcatgaagattaaggtcgttgcgccaccagagagaaagtacagtgtctggattggaggatctatcttggcatcactcagcaccttccagcagatgtggatttccaagggtgagtatgatgaatccggtccatccattgtccacaggaagtgcttctaagttttgtaattgcttttgatggtgatctacattttgcatttagttggctcttttttggcgtgccgtgtcaagtgaactcaaaagtctggtttatgtgcgggaagttagggatcattgtaggatggtgtacctgatattgacgtattattattttagcctttcaccgtatcaccaccattaagctgatgggccctaaggagatggcggtggacggacaattggtgcttaattccttccctacaatccatctttgaaccatgctgcttaaaaggatgtttggagcgggagactggattgtggtgcttttatttttttatttatttaatattcaagggttttgagaacattaatgttaatagctattattgtacgagatttttttaaaaaaaagttagtcccagtttgcgcggtcaaacttctgggaaaattatatttaataaaatcatattgtgaatcctttttgttaaaaaaaaaaaaaaaaaaaaaaaa

SEQ ID NO: 10 - DNA, 1134 bp, Gossypium hirsutum
atggccgacggtgaggctattcaacccctcgtctgtgataatggaactggaatggtgaaggccggttttgctggtgatgatgctccaagggcagtttttcccagtatcgttggtcgtccccggcacactggtgttatggttgggatgggtcagaaggatgcctatgtaggagatgaagcacaatctaaaggaggtatccttactttgaaatatcctattgagcatggtattgtgagcaattgggatgatatggaaaagatctggcatcatacattctacaacgaactccgtgttgttcctgaggagctccctgtgctactcacggaagcacctctcaaccccaaggccaatagagaaaagaagactcagatcatgtttgagaccttcaatgtacctgctatgtatgttgccatccaggccgttctctctctgtatgccagtggtcgtacaacaggtattgtgctggattccggtgatggtgtttctcacatcgtgccaatctatgaaggatatgcccttccacatgccatcctccgtcttgaccttgcaggtcgtgatctaaccgatgccttgatgaagattcttaccgagagaggttacatgttcaccaccactgctgaacgggaaattgtccgtcacatgaaagagaagcttgcttatgttgccctggactatgagcaggagctggagactgccaagagcagctcatctgttgagaagaactatgagttgcctgacggacaagttattactattgaagctgagagattccgttgcccggaagtcctcttccagccatctttcatcgggatggaagctgctggaatccatgaaactacctacaactctatcatgaagtgtgatgtggatatcaggaaggatctctacggtaacattgtgctcagtgggggttcaaccatgttccccggtattgcagaccgcatgagcaaggagatcactgcacttgctccaagcagcatgaagattaaggtcgttgcgccaccagagagaaagtacagtgtctggattggaggatctatcttggcatcactcagcaccttccagcagatgtggatttccaagggtgagtatgatgaatccggtccatccattgtccacaggaagtgcttctaa

SEQ ID NO: 11 - protein, 377 aa, Gossypium hirsutum
MADGEAIQPLVCDNGTGMVKAGFAGDDAPRAVFPSIVGRPRHTGVMVGMGQKDAYVGDEAQSKGGILTLKYPIEHGIVSNWDDMEKIWHHTFYNELRVVPEELPVLLTEAPLNPKANREKKTQIMFETFNVPAMYVAIQAVLSLYASGRTTGIVLDSGDGVSHIVPIYEGYALPHAILRLDLAGRDLTDALMKILTERGYMFTTTAEREIVRHMKEKLAYVALDYEQELETAKSSSSVEKNYELPDGQVITIEAERFRCPEVLFQPSFIGMEAAGIHETTYNSIMKCDVDIRKDLYGNIVLSGGSTMFPGIADRMSKEITALAPSSMKIKVVAPPERKYSVWIGGSILASLSTFQQMWISKGEYDESGPSIVHRKCF

SEQ ID NO: 12 - DNA, 609 bp, Gossypium hirsutum
actgcaggtttcagaagagggagtaaattgcaaagcaaatatgaataaggatttcatagctgaagaacttgacaatgacatgcaagaagaagaagaagaaggtgttgggggtgatcatggtttcccagatgatgaaaagaagaagaaaggttatggaaggagaggagccgctggtggcggtggtggggtgtcgccgcctgcttgtcaagtggaaaaatgtgggcttgatttatctgatgccaagcgataccacaggcgccataaggtgtgtgagattcatgccaaggcgccatttgtggttgttgctggtctcaggcaacgcttctgtcaacaatgcagcagattccatgagctaccggagttcgatgaagcaaaacggagctgtcgaaggcggttggccgggcataacgaaaggcgtcggaagagctcagctgaatcatcatcagcagcagaaagttcaaatcgcagagggatgatgatcagtgctcaactgaaagagtctcattacctagctgatgatcaaagagccagagtcaatccaatggcaatccatggcagttcatcttttaagcgttcccaaatcagataaggaagcttaagcgaccaaaa

SEQ ID NO: 13 - DNA, 549 bp, Gossypium hirsutum
atgaataaggatttcatggctgaagatgttgacaatgacatggaggaagaaggaggaggagaagaagaagaagagggtgctgtttatcatggtgttcaagatgatgagaagaagaagaaagcatctggaaggagagggtctagtggtggtgttggtggggtgttgccaccttcttgtcaagtggaaaaatgtggggtcgatttatcggtggccaagagataccaccggcgccataaggtgtgtgagattcatgctaaggcgccgattgtggttgttgctgggatcaggcaacgattctgtcagcaatgcagcaggttccatgagctatcagagtttgatgaagcaaaaaggagctgccgtagtcgtttggccggacacaacgaacggcgtaggaagagctcagccgaatcatccgcagaaggttcaagtcgcaagggaacaagtccacagttaaaagagggtcattgcaggcaggttgatgatcaaagagccagagttccaataacaatccctggaaattcctcttttaaatgttcccatatcagatag

SEQ ID NO: 14 - DNA, 549 bp, Gossypium hirsutum
atgaataaggatttcatggctgaagatgttgacaatgacatggaggaagaaggaggaggagaagaagaagaagagggtgctgtttatcatggtgttcaagatgatgaaaagaagaagaaagcatctggaaggagagggtctagtggcggtgttggtggggtgttgccaccttcttgtcaagtggaaaaatgtggggtcgatttatcggtggccaagagataccaccggcgccataaggtgtgtgagattcatgctaaggcgccggttgtggttgttgctgggatcaggcaacgattctgtcagcaatgcagcaggttccatgagctatcagagtttgatgaagcaaaaaggagctgccgtagtcgtttggccggacacaacgaacggcgtaggaagagctcagctgaatcatccgcagaaggatcaagtcgcaagggaacaagtccacagttaaaagagggttattgcaggcaggttgatgatcaaagagccagagttccaataacaatccctggaaattcctcttttaaacgttcccatatcagatag

SEQ ID NO: 15 - DNA, 525 bp, Gossypium hirsutum
atgcaaatggctaaaagggcatgtaaaacgacggtaaattgggttgatgatgaggaggaggaggagcagagagaagagaggatggtgaagataagggtagtgcctcgagagaggtatgagaggatgaatactttcatacactactctaactctgctgccatatccggcggcggtggtggttgtcaagctgatgattgtggtgccgatttgaaagatgcaaagcaatatcatcggcggcataaagtctgtgagccacatgccaaggatgctttcgttttggtcaaaggcattcgccaaaggttttgccagcagtgtagcagcagatttcatgaaatatcaaagtttgatggtaccaaaaggagttgccgggatcggttggccggacataatttgcggcggaggaaggtggtgcagtcagaccaagaagtggagaatgataataataataataagaataaagcatcctatggaaagggaactgacactccaatgttgcagcaatggtaccaagaatttaccaactag

SEQ ID NO: 16 - DNA, 534 bp, Gossypium hirsutum
atggaaatggctaaaagggcatgtaagacgacggtaaattgggttgatgatgatgatgatgaggaggaggaggaggagcagagagaagagaggatggtgaagataagggtagtgcctcgagagagatatgagaggatgaatactttcatacactactctaactctgctgccatctccggcggcggcggtggttgtcaagctgatgaatgtggtgccgatttgaaagatgcaaagcaatatcatcggcggcataaagtctgtgagccacatgccaaggatgctttcgttttggtcaaaggcattcgccaaaggttttgccagcagtgtagcagatttcatgaaatatcaaagtttgatggtaccaaaaggagttgccgggatcggttggccggacataatttgcggcggaggaaggtggtgcagtcagaccaagaagcggagaatgataatagtaataataagaataaagcatcctatggaaagggaactgccactccaatgttgcagcaatggtaccaagaatttaccaactag

SEQ ID NO: 17 - DNA, 426 bp, Gossypium hirsutum
atggcaacaagcaaagctgaagggaaaaggagactgaaggaaatgggggaggaggaagaagaagaagaagaggacgaggataatagtactactggtgacgatgacaagaagaaaaaaggcaaaagagggtccagtacggtggtcggaggctcctgtcttccagcctgtcaggtcgagaactgcactgccgacatgaccgatgccaaacggtaccatcggcgacataaggtgtgtgagttccatgccaaggctgctgtggttcgagttgctgggatccatcaacgcttttgtcaacaatgtagcaggttccatgagttatcagagtttgatgaaacaaaaaggagctgtcgacgacgtttggccggacacaacgagcgccgtcggaaaagttcatcggaatatcatggagaaggctcaaatttttaa

SEQ ID NO: 18 - DNA, 465 bp, Gossypium hirsutum
atggcaacaagcaaagctgaagggaaaaggagactgaaggaaatggcggaggaggaagaagaagaagaagaagaggacgaggataatagtactactggtgacgatgacaagaagaaaaaaggcaaaagagggtccagtacggtggtcggaggctcctgtcttcccgcctgtcaggtcgagaactgcactgccgacatgaccgatgccaaacggtaccatcggcgacataaggtgtgtgagttccatgccaaggctcctgtggttcgagttgctgggatccatcaacgcttttgtcaacaatgtagcagcattcaaactggtgatgctaatgcaggttccatgagttatcagagtttgatgaaacaaaaaggagctgtcgacggcgtttggccggacacaacgagcgccgtcggaaaagttcatcggaatatcatggagaaggctcaaattattaaaactagctaa

SEQ ID NO: 19 - DNA, 549 bp, Gossypium hirsutum
atgaataaggatttcatagctgaagaacttgacaatgacatgcaagaagaagaagaagaaggtgttgggggtgatcatggtttcccagatgatgaaaagaagaagaaaggttatggaaggagaggagccgctggtggcggtggtggggtgtcgccgcctgcttgtcaagtggaaaaatgtgggcttgatttatctgatgccaagcgataccacaggcgccataaggtgtgtgagattcatgccaaggcgccatttgtggttgttgctggtctcaggcaacgcttctgtcaacaatgcagcagattccatgagctaccggagttcgatgaagcaaaacggagctgtcgaaggcggttggccgggcataacgaaaggcgtcggaagagctcagctgaatcatcatcagcagcagaaagttcaaatcgcagagggatgatgatcagtgctcaactgaaagagtctcattacctagctgatgatcaaagagccagagtcaatccaatggcaatccatggcagttcatcttttaagcgttcccaaatcagataa

SEQ ID NO: 20 - DNA, 549 bp, Gossypium hirsutum
atgaataaggatttcatagctgaagaacttgacaatgacatgcaagaagaagaagaagaaggtgttgggggtgatcatggtttccctgatgatgaaaagaagaagaaaggttatggaaggagaggagccgctggtggcggtggtggggtgtcgccgcctgcttgtcaagtggaaaaatgtgggcttgatttatctgatgccaagcgataccacaggcgccataaggtgtgtgagattcatgccaaggcgccttttgtggttgttgctggtctgaggcaacgcttctgtcaacaatgcagcagattccatgagctaccggagttcgatgaagcaaaacggagctgtcgaaggcggttggccgggcataacgaaaggcgtcggaagagctcagctgaatcatcatcagcagcagaaagttcaaatcgcagagggatgatgatcagtgctcaactgaaagagtctcattacctagctgatgatcaaagagccagagtcaatccaatggcaatccatggcagttcatcttttaagcgttcccaaatcagataa

SEQ ID NO: 21 - DNA, 802 bp, Gossypium arboreum
ccttcccttgtttctcactaatcatctctgtcttcccttctcactctttgcctcttctcactgtcggctaataatggctccaaagaaggatggagtgagcaaaagggtttttaacaaaggttcttggacagctgaggaagatagaagattggctaaatatattgagattcatggcgcaaagagatggaaaacaatcgccattaaatcaggtttgaatcgatgcggcaagagttgcaggttgagatggttgaactacttgagacctaacattaagagaggcaacatatcagatgaagaagaggacttaattattaggcttcataaactgctggggaacaggtggtctttgattgctgggagacttccagggcgaacagacaatgaaattaagaactactggaattcccatttgagcaagaaaataataaaccatgatgtcagaacagaacaaacttcctcctcggaacaaattgtgcctcacaaagcatgggaaactgtccagatggaagaagaagaggtagtaaaaggaagtgatgaaattgaaaactctgaattcagcattgatgtggacgaattctttgacttcacaacggaaggttgctttagtttggattgggtgaataagttccttgaacttgatgatcaacaggatccattagcaatggtataataagtttgtaattaacatgtttccttggataaataaataaagagatgttctgagtctaaatttctaggctataaccaaatgtttcttggcctaacttttgaagcgaagaaaaaaacaacaattattggtgtg

SEQ ID NO: 22 - DNA, 597 bp, Gossypium arboreum
atggctccaaagaaggatggagtgagcaaaagggtttttaacaaaggttcttggacagctgaggaagatagaagattggctaaatatattgagattcatggcgcaaagagatggaaaacaatcgccattaaatcaggtttgaatcgatgcggcaagagttgcaggttgagatggttgaactacttgagacctaacattaagagaggcaacatatcagatgaagaagaggacttaattattaggcttcataaactgctggggaacaggtggtctttgattgctgggagacttccagggcgaacagacaatgaaattaagaactactggaattcccatttgagcaagaaaataataaaccatgatgtcagaacagaacaaacttcctcctcggaacaaattgtgcctcacaaagcatgggaaactgtccagatggaagaagaagaggtagtaaaaggaagtgatgaaattgaaaactctgaattcagcattgatgtggacgaattctttgacttcacaacggaaggttgctttagtttggattgggtgaataagttccttgaacttgatgatcaacaggatccattagcaatggtataa

SEQ ID NO: 23 - DNA, 810 bp, Gossypium hirsutum
cttcccttctcactctttgcctcttctcactgttggctaataatggctccaaagaaggatggagtgagcaaaagggtttttaacaaaggttcttggacagctgaggaagatagaagattggctaaatatattgagattcatggcgcaaagagatggaaaacaatcgccattaaatcaggtaatttactttctgttgaagagaaactccattttttggagcattggaattaacgggttattttgttttgatgtataggtttgaatcgatgcggcaagagttgcaggttgagatggttgaactacttgagacctaacattaagagaggcaacatatcagatgaagaagaggacttaattattaggcttcataaactgctggggaacaggtggtctttgattgctgggagacttccagggcgaacagacaatgaaattaagaactactggaattcccatttgagcaagaaaataataaaccatgatgtcagaacagaacaaacttcctcctcggaacaaattgtgcctcacaaagcatgggaaactgtccagatggaagaagaagaggtagtaaaaggaagtgatgaaattgaaaactctgaattcagcattgatgtggacgaattctttgacttcacaacggaaggttgctttagtttggattgggtgaataagttccttgaacttgatgatcaacaggatccattagcaatggtataataagtttgtaattaacatgtttccttggataaataaataaagagatgttctgagtctaaatttctaggctataaccaaatgtttcttggccta

SEQ ID NO: 24 - DNA, 597 bp, Gossypium hirsutum; identical to SEQ ID NO: 22

SEQ ID NO: 25 - DNA, 810 bp, Gossypium barbadense
cttcccttctcactctttgcctcttctcactgttggctaataatggctccaaagaaggatggagtgagcaaaagggtttttaacaaaggttcttggacagctgaggaagatagaagattggctaaatatattgagattcatggcgcaaagagatggaaaacaatcgccattaaatcaggtaatttaccttctgttgaagagaaactccattttttggagcattggaattaacgggttattttgttttgatgtataggtttgaatcgatgcggcaagagttgctggttgagatggttgaactacttgagacctaacattaagagaggcaacatatcagatgaagaagaggacttaattattaggcttcataaactgctggggaacaggtggtctttgattgctgggagacttctagggcgaacagacaatgaaattaagaactactggaattcccatttgagcaagaaaataataaaccatgatgtcagaacagaacaaacttcctcctcggaacaaattgtgcctcacaaagcatgggaaactgtccagatggaagaagaagaggtagtaaaaggaagtgatgaaattgaaaactctgaattcagcattgatgtggacgaattctttgacttcacaacggaaggttgctttagtttggattgggtgaataagttccttgaacttgatgatcaacaggatccattagcaatggtataataagtttgtaattaacatgtttccttggataaataaataaagagatgttctgagtctaaatttctaggctataaccaaatgtttcttggccta

SEQ ID NO: 26 - DNA, 597 bp, Gossypium barbadense; identical to SEQ ID NO: 22

SEQ ID NO: 27 - DNA, 797 bp, Gossypium hirsutum
atgtccatgaaaaaagaaggtgaaattctatacaaaaagggattatgggcaatggaggaagacaagttactcattgattatgtcaatgtccatggaaaaggacaatggaacaaaatagccaacagaacaggtttgaagagaagtgggaaaagttgtcggctaaggtggatgaattacctgagtcctaacgttaaaaagggtgatttttctgaagaagaagaagacctcgtcattagacttcataagcttcttggaaacaggtggtctttgattgcgaaacgagttccaggtcgaactgacaatcaagtcaagaattactggaatagtcatttgaggaagaaactagggatcattgatcaaaacaagacaaggatcgatttttgtcaaagttcaaagcaagtcaaagtgtgtcatgttgatgaggcagccacggatccaagtcctggacatggaacaaccactgaaaccacgggtataacagtggatcagagtaaccagcaggaagtcattgatcatcgggtcttaaacaatactactcaagaatcaatgaccagtgagagttatatcaacactttctggattcctgaccatgattatgagctaagtacacttgccatgattgaccacttccatgaatgttcttcttttcatcttagctagagactatgttattagattcgggttttatttttagatataagtatgcatctaacatggcaatgttaaatttttcaaaagatttttcatgtatttgagcagttcatgtgtttgaagattaagatatatctgaaacaaat

SEQ ID NO: 28 - DNA, 1227 bp, Arabidopsis thaliana
atctctttactaccagcaagttgttttcttgctaacttcaaacttctctttctcttgttcctctctaagtcttgatcttatttaccgttaactttgtgaacaaaagtcgaatcaaacacacatggagccgccacagcatcagcatcatcatcatcaagccgaccaagaaagcggcaacaacaacaacaagtccggctctggtggttacacgtgtcgccagaccagcacgaggtggacaccgacgacggagcaaatcaaaatcctcaaagaactttactacaacaatgcaatccggtcaccaacagccgatcagatccagaagatcactgcaaggctgagacagttcggaaagattgagggcaagaacgtcttttactggttccagaaccataaggctcgtgagcgtcagaagaagagattcaacggaacaaacatgaccacaccatcttcatcacccaactcggttatgatggcggctaacgatcattatcatcctctacttcaccatcatcacggtgttcccatgcagagacctgctaattccgtcaacgttaaacttaaccaagaccatcatctctatcatcataacaagccatatcccagcttcaataacgggaatttaaatcatgcaagctcaggtactgaatgtggtgttgttaatgcttctaatggctacatgagtagccatgtctatggatctatggaacaagactgttctatgaattacaacaacgtaggtggaggatgggcaaacatggatcatcattactcatctgcaccttacaacttcttcgatagagcaaagcctctgtttggtctagaaggtcatcaagacgaagaagaatgtggtggcgatgcttatctggaacatcgacgtacgcttcctctcttccctatgcacggtgaagatcacatcaacggtggtagtggtgccatctggaagtatggccaatcggaagttcgcccttgcgcttctcttgagctacgtctgaactagctcttacgccggtgtcgctcgggattaaagctctttcctctctctctctctttcgtactcgtatgttcacaactatgcttcgctagtgattaatgatgcagttgttatattagtagttaactagttatctctcgttatgtgtaatttgtaattactagctaagtatcgtctaggtttaattgtaattgacaaccgtttatctctatgatgaataagttaaatttatatat

SEQ ID NO: 29 - DNA, 876 bp, Arabidopsis thaliana
atggagccgccacagcatcagcatcatcatcatcaagccgaccaagaaagcggcaacaacaacaacaagtccggctctggtggttacacgtgtcgccagaccagcacgaggtggacaccgacgacggagcaaatcaaaatcctcaaagaactttactacaacaatgcaatccggtcaccaacagccgatcagatccagaagatcactgcaaggctgagacagttcggaaagattgagggcaagaacgtcttttactggttccagaaccataaggctcgtgagcgtcagaagaagagattcaacggaacaaacatgaccacaccatcttcatcacccaactcggttatgatggcggctaacgatcattatcatcctctacttcaccatcatcacggtgttcccatgcagagacctgctaattccgtcaacgttaaacttaaccaagaccatcatctctatcatcataacaagccatatcccagcttcaataacgggaatttaaatcatgcaagctcaggtactgaatgtggtgttgttaatgcttctaatggctacatgagtagccatgtctatggatctatggaacaagactgttctatgaattacaacaacgtaggtggaggatgggcaaacatggatcatcattactcatctgcaccttacaacttcttcgatagagcaaagcctctgtttggtctagaaggtcatcaagacgaagaagaatgtggtggcgatgcttatctggaacatcgacgtacgcttcctctcttccctatgcacggtgaagatcacatcaacggtggtagtggtgccatctggaagtatggccaatcggaagttcgcccttgcgcttctcttgagctacgtctgaactag

SEQ ID NO: 30 - DNA, 837 bp, Gossypium hirsutum
atggaaccccagcaacagcaaaaccaacaagctccaaatgaggattgtggtggtggcagcggcaaaggaagctttctttgcaggcaaagtagtgcacggtggactcctaccactgaccagattagaatattgaaggacctttacaacaataatggagttaggtccccgagtactgatcagattcaaaggatctctgctagacttagacagtacggaaaaatcgaaggcaagaatgtcttttattggtttcagaaccacaaggctcgtgagaggcagaagaaaaggttcaccaccacgactaatcatcaagtccccatgcaaagccctaccactgcagcaaataacaagtatcccaacataagtcctggttgttattctcctacatctctttcttcaactggtgctcttactgttgggcagataggaaactatggctatgggtctataaccatggaaaagagttttagggattgctcaatatcagcatgtggtggtagtacaagtgtaggcgaatccctgagtcacaactttggatgggttgggttcgatcctacttattcttcgtcctacacattctttgagcacaaaaaattcatggaagagagacaaaaagatgaagatgatgcagaagaagaagctgctgctccacaaattgagaccctccctctctttcccatgcacagtgaagacgataatggtttttgcagtaatattaagcccagactagacagctgctactctgggtggtacaagtctgaagatggctacactggttctcgtgcctcacttgagctcagcctcaacccatatgcaggcagatcacaagattctatctga

SEQ ID NO: 31 - DNA, 870 bp, Gossypium hirsutum
atggaaccccaacaccaacaccaacaaccacatccgaatgatgataatagtagcggtggcagtggcaaaggaagctttctttgcaggcaaagtagtacacggtggactcccaccactgaccagattagaatattgaaagacctttactacaacaatggagttaggtccccaagtgctgatcagattcagaggatctctgctaggcttagacagtacgggaaaatcgaaggcaagaatgtcttttattggtttcagaaccacaaggctcgtgagaggcagaagaaaaggttcacctccacacctgatattctccccatgcaaacacccattcttgcaaatgctaccaggaaacctgatgattctctccataatcataaatatcccaccataactcctggttatgctccttcatctcctccttcaactggtgtgcttactgttgggcagataggacactatggctatggatctatgaccatggagaagagttttagggattgctcgatatcagcgtgtggtagcagcagtggtggtgcaagccacaaccttgggtgggttggcatggatcctgcatatacaacatcatcttatgctttctttgacaagaaaaaattatttggagaagaacaagaaggtgatgaagaagaagaagaggaagaaggagcggctgttgcaccacatattgaaacccttcctctcttccccatgcacggcgaagacatcaatgctttttgcagcaatatgaagcctcaatctgcagatagctgctactctagttggtacaggtctgatgatggctacaccgcctctcgtgcttcacttgagctcagcctcaactcctacaatgccagatcacaggattctatctga

SEQ ID NO: 32 - protein, 278 aa, Gossypium hirsutum
MEPQQQQNQQAPNEDCGGGSGKGSFLCRQSSARWTPTTDQIRILKDLYNNNGVRSPSTDQIQRISARLRQYGKIEGKNVFYWFQNHKARERQKKRFTTTTNHQVPMQSPTTAANNKYPNISPGCYSPTSLSSTGALTVGQIGNYGYGSITMEKSFRDCSISACGGSTSVGESLSHNFGWVGFDPTYSSSYTFFEHKKFMEERQKDEDDAEEEAAAPQIETLPLFPMHSEDDNGFCSNIKPRLDSCYSGWYKSEDGYTGSRASLELSLNPYAGRSQDSI

SEQ ID NO: 33 - protein, 289 aa, Gossypium hirsutum
MEPQHQHQQPHPNDDNSSGGSGKGSFLCRQSSTRWTPTTDQIRILKDLYYNNGVRSPSADQIQRISARLRQYGKIEGKNVFYWFQNHKARERQKKRFTSTPDILPMQTPILANATRKPDDSLHNHKYPTITPGYAPSSPPSTGVLTVGQIGHYGYGSMTMEKSFRDCSISACGSSSGGASHNLGWVGMDPAYTTSSYAFFDKKKLFGEEQEGDEEEEEEEGAAVAPHIETLPLFPMHGEDINAFCSNMKPQSADSCYSSWYRSDDGYTASRASLELSLNSYNARSQDSI

SEQ ID NO: 34 - DNA, 1196 bp, Zea mays
gtccgagctaggtcacagaagcgctcaggaaggccgctgagatagaggcatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcacttatgtctctggaatggaaggaggaggaagtgagcatagcgttggtgcgttgctgtcattgtcctaggttagtagctagtgccagttactagtaagcatcaggcataggagtatgtagtagaagcatgcacgttgccggccagccaggctttagacgggaaaagaatttggtgcagccggctgcaaaacaggatgtttacagccccccc

SEQ ID NO: 35 - DNA, 885 bp, Gossypium hirsutum
atgtcatttattggtacccaacagaaatgcaaggcttgtgagaagactgtttatccagttgaacttttgtctgcagatggagttccttaccataaatcttgcttcaagtgcagtcattgcaaagggacactaaagttggctaattactcctcaatggaaggtgttctttactgcaagcctcatttcgagcaactcttcaaggagacgggtaacttcaacaagaatttccaatcgtctgcaaaggcagctgagaagttaactcccgagatgacgagatcaccaagcaaagctgccagcatgttttccgggacagtcgaaaaatgtgctacttgtggcaaaactgcatatccacttgagaaggtaactgtagaaggacagtcttaccacaaatcatgtttcaagtgctctcatggtggctgctctttgagtccatcaaattatgcagcacttgaaggcattttgtactgcaaacatcacttttcccagctcttcaaggagaaaggaagctacaatcatcttatcaaatccgcatcaatcaagcgtgccgctgcatccgttcctgaagcttgaatacctatttcatgttttttgtttattcccatgtttgccggtgtgtttttttattgattttttggatttatgctcgggaggggaactggaaagaaagaacttgggtttctttccgtttttttgaggccttggtatttgattgatttaagtagaagtgaatgttgtaacaggactctgtgcaagattttttatgcaatccaaatttggctcatttcgaatgaatatttttataaataataaaaatgttagatacactacttggcaataaaaaaaaaaaaaaaaaaaaaaaaaaaaaaaaaaaaaaaaaaaaaaaaa

SEQ ID NO: 36 - DNA, 570 bp, Gossypium hirsutum
atgtcatttattggtacccaacagaaatgcaaggcttgtgagaagactgtttatccagttgaacttttgtctgcagatggagttccttaccataaatcttgcttcaagtgcagtcattgcaaagggacactaaagttggctaattactcctcaatggaaggtgttctttactgcaagcctcatttcgagcaactcttcaaggagacgggtaacttcaacaagaatttccaatcgtctgcaaaggcagctgagaagttaactcccgagatgacgagatcaccaagcaaagctgccagcatgttttccgggacagtcgaaaaatgtgctacttgtggcaaaactgcatatccacttgagaaggtaactgtagaaggacagtcttaccacaaatcatgtttcaagtgctctcatggtggctgctctttgagtccatcaaattatgcagcacttgaaggcattttgtactgcaaacatcacttttcccagctcttcaaggagaaaggaagctacaatcatcttatcaaatccgcatcaatcaagcgtgccgctgcatccgttcctgaagcttga

SEQ ID NO: 37 - DNA, 2268 bp, Agrobacterium tumefaciens
atgtcagcttcacctctccttgataaccagtgcgatcatctcccaaccaaaatggtggatctgacaatggtcgataaggcggatgaattggaccgcagggtttccgatgccttcttagaacgagaagcttctaggggaaggaggattactcaaatctccaccgagtgcagcgctgggttagcttgcaaaaggctggccgatggtcgcttccccgagatctcagctggtggaaaggtagcagttctctccgcttatatctatattggcaaagaaattctggggcggatacttgaatcgaaaccttgggcgcgggcaacagtgagtggtctcgttgccatcgacttggcaccattttgcatggatttctccgaagcacaactaatccaagccctgtttttgctgagcggtaaaagatgtgcaccgattgatcttagtcatttcgtggccatttcaatctctaagactgccggctttcgaaccctgccaatgccgctgtacgagaatggcacgatgaaatgcgttaccgggtttaccataacccttgaaggggccgtgccatttgacatggtagcttatggtcgaaacctgatgctgaagggttcggcaggttcctttccaacaatcgacttgctctacgactacagaccgttttttgaccaatgttccgatagtggacggatcggcttctttccggaggatgttcctaagccgaaagtggcggtcattggcgctggcatttccggactcgtggtggcaaacgaactgcttcatgctggggtagacgatgttacaatatatgaagcaagtgatcgtgttggaggcaagctttggtcacatgctttcagggacgctcctagtgtcgtggccgaaatgggggcgatgcgatttcctcctgctgcattctgcttgtttttcttcctcgagcgttacggcctgtcttcgatgaggccgttcccaaatcccggcacagtcgacacttacttggtctaccaaggcgtccaatacatgtggaaagccgggcagctgccaccgaagctgttccatcgcgtttacaacggttggcgtgcgttcttgaaggacggttttcatgagcgagatattgtgttggcttcgcctgtcgctattactcaggccttgaaatcaggagacattaggtgggctcatgactcctggcaaatttggctgaaccgtttcgggagggagtccttctcttcagggatagagaggatctttctgggcacacatcctcctggtggtgaaacatggagttttcctcatgattgggacctattcaagctaatgggaataggatctggcgggtttggtccagtttttgaaagcgggtttattgagatcctccgcttggtcatcaacggatatgaagaaaatcagcggatgtgccctgaaggaatctcagaacttccacgtcggatcgcatctgaagtggttaacggtgtgtctgtgagccagcgcatatgccatgttcaagtcagggcgattcagaaggaaaagacaaaaataaagataaggcttaagagcgggatatctgaactttatgataaggtggtggtcacatctggactcgcaaatatccaactcaggcattgcctgacatgcgataccaatatttttcaggcaccagtgaaccaagcggttgataacagccatatgacaggatcgtcaaaactcttcctgatgactgaacgaaaattctggttagaccatatcctcccgtcttgtgtcctcatggacgggatcgcaaaagcagtgtattgcctggactatgagtcgcaggatccgaatggtaaaggtctagtgctcatcagttatacatgggaggacgactcccacaagctgttggcggtccccgacaaaaaagagcgattatgtctgctgcgggacgcaatttcgagatctttcccggcgtttgcccagcacctatttcctgcctgcgctgattacgaccaaaatgttattcaacatgattggcttacagacgagaatgccgggggagctttcaaactcaaccggcgtggtgaggatttttattctgaagaacttttctttcaagcactggacacggctaatgataccggagtttacttggcgggttgcagttgttccttcacaggtggatgggtggagggtgctattcagaccgcgtgtaacgccgtctgtgcaattatccacaattgtggaggcattttggcaaagggcaatcctctcgaacactcttggaagagatataactaccgcactagaaattag

SEQ ID NO: 38 - protein, 755 aa, Agrobacterium tumefaciens
MSASPLLDNQCDHLPTKMVDLTMVDKADELDRRVSDAFLEREASRGRRITQISTECSAGLACKRLADGRFPEISAGGKVAVLSAYIYIGKEILGRILESKPWARATVSGLVAIDLAPFCMDFSEAQLIQALFLLSGKRCAPIDLSHFVAISISKTAGFRTLPMPLYENGTMKCVTGFTITLEGAVPFDMVAYGRNLMLKGSAGSFPTIDLLYDYRPFFDQCSDSGRIGFFPEDVPKPKVAVIGAGISGLVVANELLHAGVDDVTIYEASDRVGGKLWSHAFRDAPSVVAEMGAMRFPPAAFCLFFFLERYGLSSMRPFPNPGTVDTYLVYQGVQYMWKAGQLPPKLFHRVYNGWRAFLKDGFHERDIVLASPVAITQALKSGDIRWAHDSWQIWLNRFGRESFSSGIERIFLGTHPPGGETWSFPHDWDLFKLMGIGSGGFGPVFESGFIEILRLVINGYEENQRMCPEGISELPRRIASEVVNGVSVSQRICHVQVRAIQKEKTKIKIRLKSGISELYDKVVVTSGLANIQLRHCLTCDTNIFQAPVNQAVDNSHMTGSSKLFLMTERKFWLDHILPSCVLMDGIAKAVYCLDYESQDPNGKGLVLISYTWEDDSHKLLAVPDKKERLCLLRDAISRSFPAFAQHLFPACADYDQNVIQHDWLTDENAGGAFKLNRRGEDFYSEELFFQALDTANDTGVYLAGCSCSFTGGWVEGAIQTACNAVCAIIHNCGGILAKGNPLEHSWKRYNYRTRN

SEQ ID NO: 39 - DNA, 1938 bp, Gossypium hirsutum
atgattagttggaacgatctttacaccgttttaacggcggtgatcccactgtacgttgctatgatcttggcttacggctccgtccgttggtggaaaatattcacccccgaccagtgctcgggtatcaaccgctttgtcgccatatttgccgttcctctcttgtctttccacttcatttccaccaatgacccttatgccatgaacttcaggttcgtagcagccgacacccttcaaaagctcatcatgctcttcgttcttggattgtggactaatttaacaaggaacggaagcctagaatggatgatcaccattttctctttatctactctccccgacactctagtcatgggcattcctcttttaatcgctatgtacggtccctactccgggatgctcttggtccaagtcgtggttctccagtgtatcatttggtacacccttcttcttttcctttttgagtaccgcggtgccaaaatcctcatcatggagcagtttcccgaaactgcagcttccattgtttctttcaaagttgattccgatgtggtttcacttgacggccgcgacttcctcgaaaccgacgctgaaatcggggaagacggcaagctacacgttaaggtcaggaaatctaatgcttccaggaggtctttggggccttgttcccttcctgcattgactcccaggccttccaacctcaccggagctgaaatctacagtttgagctcttcaaggaaccccacgccaagaggttccaatttcaacaactccgacttttactccatgatgggtgttcaaggatttccggcaagacactccaatttcggtccagctgatttatactctgttcaatcctctagaggacctactccaaggccatccaattttgaagaaaacaatacagtaatgtctccacgatttggattttatccagcacagactgttccttcatcctaccctgctccaaaccctgaattctcatccgtcacgaagaacgctaaagctacccaacaacaacagcagcagccagttcaaccacagcagcagcctaaggagaaggagaataataaagagaatcatgatgccaaggaattgcacatgtttgtgtggagttcaagtgcctcgccggtttcagaaggcggaggtctccatgtctttggtggtacagatttcggagcgtccgagcaatctggacggtctgagcagggtgctaaagagataaggatgttggtcgcagatcaccctcaaaacggggaaaacaaaggcatgacaggcagtggtgacgttaatggagaggactttagctttgctggaagagatggggaagaggagagagaaaaggaaggacccaatggtctcaataagttggggtctagttcaacggccgagttgcaccctaaagccgccggagggcctgagtctggcgtaggcaaacaaatgccgccggcgagtgtaatgacccgcttaatcttgatcatggtctggcgcaagcttatacgaaaccccaacacatattccagtctcatcgggctggtttggtccctaattgctttcaggtggcatgtgagtatgccgaaaataatagagaagtctatctccatcctgtcagatgctggactaggaatggccatgtttagcttaggtctgttcatggcactgcaacccaagatcatcgcttgtgggaactctgtagctacatttgccatggccgttaggttcttaaccggtccggctgtcatggctgccgcttcgattgcagtagggttgcgcggcacactcctccgtgtcgccattgttcaggcggctctgccacaaggaattgtaccatttgtgttcgccaaggaatacaatgtccaccctgcaattcttagcactgcggttatctttgggatgttgatagcattaccaataacgctggtgtactacattcttctaggattgtga

SEQ ID NO: 40 - DNA, 1086 bp, Gossypium hirsutum
atggagtcaaacgcggggacattggagaccagatctaatagaacggcgttggaaagggaggaagtagcggcacttgaatcgctacaatttaccgacgaaatccaccgtttgatgtcggctccggcgaccgacaatgccagttccttcacggctttgcttgaactccctgctccccaagctgttcagcttattcactctccggattcggctaagctcattgccactccagctccgaacgttgaagatttcaaaggcgggtttcatttcccttctaatggtgggctaattgaacgagcagccaggttctccgtgttcgccggcgaaggtaaaaacaagagcgactcgcctgaagcgacgtcgaacaattcaagtgcaaatctccagaaggcagtgaagagtgaaccggctgagaccgagtcgtctcagccgttaatttccgatccaacggtggagaaccggggtatcaagagaaaagaccgagaaaaggtgaaagggcagacgaagaaaagcaaaacaacggcgaaagagagctccgacgacgccgagaagctgccttacgttcacgttagagctcgccggggacaagcaacggatagccatagcttagctgagagagcaaggagagagaagattaatgcacgtatgaagctcctacaggagttagtaccaggatgcaataagatttcgggcacagcgttagttttggatgaaatcatcaaccatgtgcagtccctacaacgtcaagtggagttcttatcaatgacactcgctgcagtgaacccaagaattgatttcaactttgatagtgtattcacggcagaaagtggatctttaatggacagtaacttttctggcatggttatgcctgtgatgtggcccgaggttcaactgaacagcaacagacaacagtatccgcagcaattgcattttgatgcatttcagcaacctgtttgggggagagaagaagtctgcaataattacttaactccggagaactcgcttttaagctatgattcctctgcaaactcaggacatccttttgggaacactcctcgacttaacacttgtgtgaactccactcactaa

SEQ ID NO: 41 - DNA, 1742 bp, Gossypium hirsutum
aaaaaaaattccctgtttattattaaaccagacaaacccagctacgttgctttctttctacttcaaatggctgtgaaattaatatctctcaataacaattaacaagttttctgttagcttgagctgaccgtcattgaagaactaaatcaaagcttgtaaaaaaatatttgaagttgttaagtgttgaaattggggtgaattaaaggtgtgggtgtttcttattttcaaagaattgaattggtttttctttcagaatttggattttaggtttgttcttcgtttttctgctatggagtcaaacgcggggacattggagaccagatctaatagaacggcgttggaaagggaggaagtagcggcacttgaatcgctacaatttaccgacgaaatccaccgtttgatgtcggctccggcgaccgacaatgccagttccttcacggctttgcttgaactccctgctccccaagctgttcagcttattcactctccggattcggctaagctcattgccactccagctccgaacgttgaagatttcaaaggcgggtttcatttcccttctaatggtgggctaattgaacgagcagccaggttctccgtgttcgccggcgaaggtaaaaacaagagcgactcgcctgaagcgacgtcgaacaattcaagtgcaaatctccagaaggcagtgaagagtgaaccggctgagaccgagtcgtctcagccgttaatttccgatccaacggtggagaaccggggtatcaagagaaaagaccgagaaaaggtgaaagggcagacgaagaaaagcaaaacaacggcgaaagagagctccgacgacgccgagaagctgccttacgttcacgttagagctcgccggggacaagcaacggatagccatagcttagctgagagagcaaggagagagaagattaatgcacgtatgaagctcctacaggagttagtaccaggatgcaataagatttcgggcacagcgttagttttggatgaaatcatcaaccatgtgcagtccctacaacgtcaagtggagttcttatcaatgacactcgctgcagtgaacccaagaattgatttcaactttgatagtgtattcacggcagaaagtggatctttaatggacagtaacttttctggcatggttatgcctgtgatgtggcccgaggttcaactgaacagcaacagacaacagtatccgcagcaattgcattttgatgcatttcagcaacctgtttgggggagagaagaagtctgcaataattacttaactccggagaactcgcttttaagctatgattcctctgcaaactcagtgtcgttgcactcgaatcaattgaaaatggagctatgaagtccgaaagaagctcgaaatggttagatttatcaaaaaaaaaaaaaaagttctaatgatagctagtattgtatatatattatatatatcagaaattagcagggaagtagaagagaggcctcaccatatatatatatcatttggtttccatgtgttctatttagccgacttcagatggaaaccttcgatttgtaaggactggagcatgaaatctcagtatgttgttattatatatatatgttatatactaactcggagtcattcaatcaaaatactcggtttgaggttgattgattcatattgtaaaatcatagaacaaactatgatgattcaaactattatatgaagtcttggcttcttttgtttctattatttgttgtcatct

SEQ ID NO: 42 - DNA, 1068 bp, Gossypium hirsutum
atggagtcaaacgcggggacattggagaccagatctaatagaacggcgttggaaagggaggaagtagcggcacttgaatcgctacaatttaccgacgaaatccaccgtttgatgtcggctccggcgaccgacaatgccagttccttcacggctttgcttgaactccctgctccccaagctgttcagcttattcactctccggattcggctaagctcattgccactccagctccgaacgttgaagatttcaaaggcgggtttcatttcccttctaatggtgggctaattgaacgagcagccaggttctccgtgttcgccggcgaaggtaaaaacaagagcgactcgcctgaagcgacgtcgaacaattcaagtgcaaatctccagaaggcagtgaagagtgaaccggctgagaccgagtcgtctcagccgttaatttccgatccaacggtggagaaccggggtatcaagagaaaagaccgagaaaaggtgaaagggcagacgaagaaaagcaaaacaacggcgaaagagagctccgacgacgccgagaagctgccttacgttcacgttagagctcgccggggacaagcaacggatagccatagcttagctgagagagcaaggagagagaagattaatgcacgtatgaagctcctacaggagttagtaccaggatgcaataagatttcgggcacagcgttagttttggatgaaatcatcaaccatgtgcagtccctacaacgtcaagtggagttcttatcaatgacactcgctgcagtgaacccaagaattgatttcaactttgatagtgtattcacggcagaaagtggatctttaatggacagtaacttttctggcatggttatgcctgtgatgtggcccgaggttcaactgaacagcaacagacaacagtatccgcagcaattgcattttgatgcatttcagcaacctgtttgggggagagaagaagtctgcaataattacttaactccggagaactcgcttttaagctatgattcctctgcaaactcagtgtcgttgcactcgaatcaattgaaaatggagctatga

SEQ ID NO: 43 - DNA, 1262 bp, Gossypium hirsutum
caaagcttgtaaaaaaatatttgaagttgttaagtgttgaaattggggtgaattaaaggtgtgggtgtttcttattttcaaagaattgaattggtttttctttcagaatttggattttaggtttgttcttcgtttttctgctatggagtcaaacgcggggacattggagaccagatctaatagaacggcgttggaaagggaggaagtagcggcacttgaatcgctacaatttaccgacgaaatccaccgtttgatgtcggctccggcgaccgacaatgccagttccttcacggctttgcttgaactccctgctccccaagctgttcagcttattcactctccggattcggctaagctcattgccactccagctccgaacgttgaagatttcaaaggcgggtttcatttcccttctaatggtgggctaattgaacgagcagccaggttctccgtgttcgccggcgaaggtaaaaacaagagcgactcgcctgaagcgacgtcgaacaattcaagtgcaaatctccagaaggcagtgaagagtgaaccggctgagaccgagtcgtctcagccgttaatttccgatccaacggtggagaaccggggtatcaagagaaaagaccgagaaaaggtgaaagggcagacgaagaaaagcaaaacaacggcgaaagagagctccgacgacgccgagaagctgccttacgttcacgttagagctcgccggggacaagcaacggatagccatagcttagctgagagagcaaggagagagaagattaatgcacgtatgaagctcctacaggagttagtaccaggatgcaataagatttcgggcacagcgttagttttggatgaaatcatcaaccatgtgcagtccctacaacgtcaagtggagttcttatcaatgacactcgctgcagtgaacccaagaattgatttcaactttgatagtgtattcacggcagaaagtggatctttaatggacagtaacttttctggcatggttatgcctgtgatgtggcccgaggttcaactgaacagcaacagacaacagtatccgcagcaattgcattttgatgcatttcagcaacctgtttgggggagagaagaagtctgcaataattacttaactccggagaactcgcttttaagctatgattcctctgcaaactcagtgtcgttgcactcgaatcaattgaaaatggagctatgaagtccgaaagaagctcgaaatggttagatttatcaaaaaaaaaaaaaaaaaa

SEQ ID NO: 44 - DNA, 1068 bp, Gossypium hirsutum; identical to SEQ ID NO: 42

SEQ ID NO: 45 - DNA, 2260 bp, Zea mays
cttccctaacctttgcactgtccaaaatggcttcctgatcccctcacttcctcgaatcaatctaagaagaaactcaagccgcaaccattaggggcagattaattgctgcactttcagataatcaaccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaa

SEQ ID NO: 46 - DNA, 2133 bp, Zea Mays
atggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaa

SEQ ID NO: 47 - DNA, 3665 bp, Gossypium hirsutum
gttaagttcaaagaaatggctgatcgtgtgatcactcgcgtccacagtctccgtgagcgtttggatgagacccttcttgctcacaggaacgagattttggccttgctctcaaggtaattcgaatctgatccatcaatgactgttgccctgcatggtgtttttcttttgagatgtttacattttgtttataaaattgttggtttttttttgtaggatcgagggcaaaggaaaaggaattctgcaacaccatcaaattattctagagtttgaagctatccctgaagagaacagaaagaagctcgctaatggtgcattttttgaagtattgaaggctagtcaggtataaggaaaacccattttatccatttcttttttcattattttgggggattacaaggtgttgattttagggtcatttttgtggttataggaagcgatcgtgttgcctccatgggttgcacttgctgttcgtccaaggcctggtgtttgggagtacattagagtgaatgttcacgcccttgttgttgaggaactcactgttgctgagtatctccacttcaaggaagagcttgttgatggaaggtaactatatgatctgtttgtatatgtatatttgagttcatctgatctgttggttggtttttaacaatgtttctttgcattatttgtagttcaaatggaaactttgttttggaattggattttgagcccttcaactcatcattcccccgcccaactctttcaaaatccattggtaatggtgtggagttcctaaatcgtcacctttcggcaaaattgttccatgacaaggagagcatgcaccctttgctcgaattcctcagagtccattgtcacaagggcaaggtatgcctcatttgcttttattgcaaaaaaaatctgtcagaatttgtcagtgaatagatcagacagatttcttgcatgctgatactaagatttactagattcctagtactgtctgtttttttttctggatttaaaatgctgagttgttctttttgcagaacatgatgttgaatgacagaattcagaacttgaatgctcttcaacatgttttgaggaaagcagaggagtatcttggtaccctacctcctgagacaccatgtgccgaattcgaacaccggttccaggaaatcggtttggaaagaggttggggtgacaccgcagaacgcgtgctcgagatgatccaactccttttggatcttcttgaggcacctgatccttgcacccttgagaagttccttgggagaatccctatggtgttcaatgttgtgattctcactccccacggatacttcgctcaagacaatgttttggggtatcccgacaccggtggccaggttgtttacatcttggatcaagtccgagctttggagaatgagacgctcctccgtataaagcaacaaggactcaacatcacccctcgaatcctcattgtgagactgctagtagttttaatccttcctttcggctttttactattttgtatgctcatatctctgtttcctctttcagattactagacttcttcctgatgctgtcggaacaacatgcggtcaacgacttgagaaagtatacggaacagagcactcggatattcttcgagtacccttcagaacagaaaagggaattgttcgtaaatggatctcaagatttgaagtctggccatacttggaaacctacacagaggtgaaactcatgagccattgattcattttgtatgatggtagatcacattagttttatttacgagtaaatatgtttgtttcataggatgttgctcatgaaatctccaaagagttgcaaggcaagccagatctgatcatcggaaactacagcgacggcaatatcgtcgcctccttgctcgcacataaattaggtgtcacacaggtttgttagaaactaatttgccggatcttgtatcttctcgtatattcggtcgggtcggattaccttacaatgacttggtttttcacccttgtgtagtgcaccatcgcccatgctttggagaagacaaaatatccagattcagatatctattggaagaagcttgaagacaaataccatttctcttgccaatttacagctgatctttttgcaatgaaccatacagatttcatcatcaccagtactttccaggaaattgcaggaaggtaataaatagatgatttaattctcagtacttgagatattttattaatgctcggcggataatgaagatcattaacattaataaaattctcaaatggttttcagcaaggacactgttggtcaatacgagagccacactgctttcactcttcctggtctctaccgtgttgtacatggtatcgatgtgtttgatcccaaattcaacattgtttcccctggtgctgatatggagatatacttcccttacaccgaagagaagcggaggttgaagcatttccatcctgagatcgaagaccttctttacagcaaagttgagaatgaagaacacttgtaagtgcaagcttttttatatgttttagcagaatccgattgtgggaacatatcccgtctgaattaatgatttgtccctgattttcctacagatgtgtgctcaatgaccgcaacaagccaattctgttcacaatggcaaggcttgatcgtgtcaagaacttaaccggactcgtcgagtggtacggcaagaacgcaaagttgcgtgagttggctaacctcgtagttgtaggtggtgataggcgaaaggaatctaaagatttggaagagaaggctgaaatgaagaaaatgtttgagctgatcgacaagtacaacttgaacggccaattcagatggatatcatctcaaatgaacagaatccgaaatggtgaactttaccgatacatttgcgacacgaaaggtgcctttgtacagcctgcattgtatgaagcctttggattgacagttgtggaggcaatgacttgcggtttgccaacattcgcaacctgtaacggtggaccagccgagattattgtccatgggaaatctggtttcaacattgatccttaccatggtgatcaagctgctgacatactcgtcgatttctttgaaaagtgtaagaaagatccatctcactgggataagatctcccaaggaggcttgaaacgaatagaggagaagtaagctctcacacacacacaataaagaaaaaaccattttccacttgggtttatattgtttagtatttgttgtaacgattataaattgtctgaatcaggtatacatggaagatttactcggagagactattgaccctgacaggagtgtatggattctggaagcatgtttccaaccttgaacgccgtgagagtcgtcgttaccttgagatgttttatgctcttaagtaccgtaagctggtaattcacatggtcacatccccattctgtataaatgtcctttatttggctttaattacattgttggtgatttgtaaaatgcaggctgaatcagttccattggcagaggagtaaattgaagctgttaaataacattgggccggtttttcttggagaataatattctgttttgtaatttcaattggagaagctcctttgtatttcatcttgtcttttccttttccttttttcgccggcattgtttgaacatggggttgtgcgcccgtcaattcc

SEQ ID NO: 48 - DNA, 1450 bp, Gossypium hirsutum
tgtcgtgtgggtatagaaccactaccttagaagatgttgcattgcaactcaggctctcaattgacggtggttatgcaatcacagattcaagtataactcgggctccagttgacaatggttatgtagtcacgggttcaagtttcatgccatcggaagagaatgttttataaacctcatacataagtttgagatcataatcatcggggaaaaatagggctttccagtataatcaatttattatttttctccatctccattaaagtagacatcaccgtttgaaacaggagtcgaggcggcagtgattgctgtgattaaggaactcgattaagaattacaatcgttgctgctccaactcgagtttcagctttttctccaccgtcacaatcattcctcccccctgaaagtcttcttgcacccaacactgtcacaggttatggacaatgaggttaaagatactacctttggtggagatgaagaaaaataataagttgattatactgaaaaacccttttggttttccctatgatggtctcaaacttatgtatgagatttataaagtatcctcttttgatatcatgcaatttgaacccatcaccacataactaccgccaacttaggcctaagttatacatcaacttgtgatcgtttaaccaacgtcaactgagagctctagctatacttcaacttgtgactgtgtagacaccatgaactaggagcctgagttgaaatgtgatgagcttctaaggtgatggtttcatctgcacacggtagcatgaaatgtgtgagtctttgagcgtcattgcatcaccgtgaactcaaacataccttaaaattatcataatcggtatctctttcacaaacacaaataaatttttctcctcaaatttccaacactcaacacaaaattttttttttttagaaacaaaaaacgtttcctacacgtattttctgtttctttctcccaaatgcacctattttcttaaatgacaaaaaaaaatctaaattcataattaacttttaaattaatctttttttatctaattaacccgaaagttaagtgatatttattctaaaaatagtaaaacgtatttcttaatgtcaaggctctgcgtggatgcggtgaacaatccatagccgacagcagctaccggcttcgtgtcgccaccttatcttatcttgtcttggatttacctgtctaattgattaaaatacttaaacaaacatcctaagccttacatgtttgccatatggctgaaacgtgtaagcaattcaatgatccaaggagcatacgtgtcagatttacttgtatacgtggcagctgagcaactatcatttgggtcctattaatggcaagaattttggagttcgagcatcaacttcatcagcaagcaaatataccctgaaaaccctttcactaaatattgaagatgcagtcaatgaaggatgctgc

SEQ ID NO: 49 - DNA, 1679 bp, Gossypium barbadense
catataaaagtttaggggattggagaggttgccacccaatgtcacggcattcccatcagtctagctgaaagtgtgatatttatagatcatttatcaaatgaagttatcagtgaaatgccaactttagtgcttatcaaagtgataataagcactgagccaatgcatgttgacattagtctttgaaatctaaaccagaaattatctcacataatgtttacattgtaaaatcacaggctaactatcactcttaagaatccaacaaggttttcattagggaaatgatagccaagatgaaccacaccatattaagagcaggttccatattgggaagttttaggactaatgtatcatctatgaggttccaaatgagttttaggattagtgtatgaaacgaaattttatggctacaatacatttccaagtactgtttccttttaaacatcccaataaattggaaggacatatatctttgtctacggttcagaccacagagctatgggttattcttgtattaaaattgttcgaattcatcttataattatattcaatacatataattagtgagtcattttagatcatttctatattcatatttgttgagggagattgatttctttggaagaaattttcaaagtgtcatccttaaggttgccgatttggtccctcaacaatggaaggtattatcctcttagggagggttttgtcgcccctccccgtcctcaaggatgccgacccgatccctcaacaatggaaggtattgtccctcaacagatgacctatccgtaggctaggggttcgaatcctaccaagggtgggatgctcacgtcacctcttggtcagcagtcgtaaatccacgtacggttaaggtgtccccttccgtgaattggacacggctctttgtggaggagggacaaaatcctcccctgaggactataccttccgttgttgagagactgaatcggcatccccaagaatgacacttcaaaaatttctcctaaaagaactgatctccctcaataatattgtgtttgtattttgttttcttgtgttgtgttatatttttaatctttttttcaaatcatatttgtgcgttttatccaaaattaataaattatcaaatcttgataagagggatgtcttgggagacagatactaatgtttaagcaaacattaatctcatgcctataccaacctattgattttatatttatatacgtctcctgtggtaagtgaagatggtcccaattcatttatctctcttcattggattacatctccacttggacttacccattaatcagtaatttcttttctccctaaaagttgataattatcgtccaatatcctagtcttgtgaacaaaattatctgatgatgataaccaaatataatatgcctgattgtaatccaaagtgacccgcccatgttccatgagttgaagtcctcgttagaaagaccatttcaagttcagcattgtttcagcaattcaaaagttaataattaatacaaacatataaatgcacctgctgtcatcaactaacctcatgcaggttcacctgtttcatttacttcacctagttagtgctttatattgatcatccccctcattcttctcaactcacacatcttccaatctttcattctttgctgttgaagtagtaatatctatagcatacagag

SEQ ID NO: 50 - DNA, 1351 bp, Gossypium barbadense
atctacctcttattgaatttgcatacaataatagctttcaattgagtatcaaaatggcaccttacgaagctttgtacagtcgtaaatgtcatacacctttgtactagactgagctcagtgaaaataagatacacgtggtcgatttgattagagaaactgaacagaaagtgaaagtgatccatgaaagtttgaaaacaacattagatcgtcagaaatcgtatgcagatttgaaatgtaaagacatagagtttcagatcggggatagagtgtttttgaaagtctcaccgtggaggaaaataatcagattcggtcgtaaaggcaaattgagtccgagatttgttggaccatacaagattatagagaggattggaccggttgcgtatagactgctattaccacctgagctggaaaagatccataatgtacttcatgtttcgatgctttgacgatatagatccgaccccttgcatgtcattagtccatcagagattgagattcattcaaatttgacttacgaggaagaactgattcatattttggctcatgcaattaaagagttacaaaataataaaattttgttggttaaagtactgtgggataaacatggtgttgaggaagcaacgtgcgagccagaggatacaatgaaagagtgttatccgaacctattcaccggtaagatttttggggacgaaaatccctaaggggggagagttgtagcagaccgattagaccctaatcggaacggtgattttgggaccacaaatccgagtcagaaaaatatttttccgtgtttattatatgtgaatttgcatgtgtgaacgtttcctacgaaaatttaattgtttgtgtgcgtaatttgataaaaaggacttaatcgcgtaaattgaaaatttgataatttaaatcataaaggctaaatttttagtggctttttaatatgaggtgtttatgttgcaaattgcccattaatcagtaatttcttttctccctaaaagttgataattatcgtccaatatcctagtcttgtgaacaaaattatctgatgatgataatcaaatataatatgcctgattgtaatccaaagtgacccgcccatgttccatgagtcgaagtcctcgttagaaagaccatttcaagttcagcattgtttcagcaattcaaaagttaataattaatacaaacatataaatgcacctgctgtcatcaactaacctcatgcaggttcacctgtttcatttacttcacctagttagtgctttatattgatcatccccctcattcttctcaactcacacatcttccaatctttcattctttgctgttgaagtagtaatatctatagcatacagag

SEQ ID NO: 51 - DNA, 2080 bp, Gossypium hirsutum
acgtcgcatgctcccggccgccatggcggccgcgggaattcgatttgtgtcaaagacgactacttgagactgtcaaaacctatacaacaaaaaaaaattaaaatcattctcttaatttacccatttgaaatggatgccacacataccatttgaaatatcttaaagagaactttcatttgacaccaccaatgttgtcggaacctcaccagaatttagtaactatactaacccaaatgaaatacatgactttcacgaaaaatagattaaaacacaagaaatctaaaccaaagttatccttaatgtctaaaatccactagttatttgcccttcctttttctttcttcctcttctctatggttagcttctaccaaattgtcaagatcatcatctaacataaaaataccgtaaatgattcttttctctttttttctagtctattctttttattagaaaaaactataacctacataataaagttgagcttgaaaagaaaattggctaatggaccacatatttaaataatacccatttaaaaatcatgaatattacaaatgtaataacacctgttaaaggaaaggaggtcacaaacaaaagtgatttgtacagagttttttatcaatgcaaagttggcgatatgatatagcattgaataaaaccaataaccatttctttctctctctatatatatacacgtctttaggatcttttatgatgtacaaatttttcataattttaatttttaaattatcgtggaggtgttacatgaggagaagatccctataacactcaagccaaagccagatgagtcatgaaatgatgtttagtatgttgcataatcctaaatacctagtaaatatacaaggaaatgtcattatttgtttactattttactggaaaaatattcctgttgtatctatgtggctatgccatcaacttgttatcatctttcatacaatgttttcacaatgtgaagcacaaggctatcaacctttgtaaaaaattgtatctatcttgccttttcttcttaactgattggggtataagtgcaaattctatgttacatggactcgtctatgagtattgaatatgggtaacacgaaataagcaaaccctagtaaataagggacctcctaatggtgtgttgtaagcttaggtggaagtgtagaaaagaggaaagggaaattgattgtcccaaaaaagtaagtgatggatttccgaatttagataaatgtgcatttgctaaggcattacggcccctacgtgcacggatgtgatatggcagttagttgttggtcataaatatctttgcattttctagtttgagttcaagtagtggggtttggatgagtgattgggtgcggtgcagtgtagtgcaatgcgtttagtttactttttatctcacactacagtatcattacaatatataatctcaccgtcactgctatttttacactaaccgcaagaaaatgtaccatctcatacaaactaagccttagacttataaacttttaaaactcgcatctaattcatttaattttaaaaaaattaatagattatatttgaattacaacatttgtaatgtattctcttaatccggaagcgatgaactcttttgtttatatatagaaaaaagaacacaataatttgggttatggattttttttaactttaacatttaccattcattttaatcctggaattaatttagcatgaatctaattaagagagctcacaaaatatgtgaagatgtgtgcaaaagctattaagagtaatttatccaaatctgccatccatcatcatctcatcattacctccatatatacaaccagaatcctacacttacagctaagaaaggaatatatatatatttaaaacagcatttgtgtttcaaaaaaatctaatccatatactgcaaaatccttgtgcatcttcttcagagcaaacggcaacaactccaacaaatgtccatgaaaaaagaaggtgaaattctatacaaaaagggattatgggcaatggaggaagacaagttactcaatcactagtgaattcgcggccgcctgcaggtcgaccatatgggagg

SEQ ID NO: 52 - DNA, 12578 bp, artificial
  Synthetic construct stunt 7 promoter
gcggccgcttgcatgcctgcagtaattaatagtaattatgattaattatgagataagagttgttattaatgcttatgaggaataaagaatgattaatattgtttaattttattccgcgaagcggtgtgttatgtttttgttggagacatcacgtgactctcacgtgatgtctccgcgacaggctggcacggggcttagtattacccccgtgccgggatcagagacatttgactaaatattgacttggaataatagcccttggattagatgacacgtggacgctcaggatctgtgatgctagtgaagcgcttaagctgaacgaatctgacggaagagcggacatacgcacatggattatggcccacatgtctaaagtgtatctctttacagctatatcgatgtgacgtaagatgctttacttcgcttcgaagtaaagtaggaaattgctcgctaagttattcttttctgaaagaaattaatttaattctaaattaaattaaatgagtggctataaatagtgtcgatgctacctcacatcgtattcttcttcgcatcgtctgttctggttttaagcgatacgtagaattcctcgagggtaccatcgatcctagggagctcgttaacgtttagcgggggaaaaaggacagttgatctgttgctgtttgcaattttttaaagggtatgttgtcagatgcatgttgtaatgcttgttcatcaacacattatatgacttgcagttgctgatgatggaaacttaaagcttaatactacttttgtttattcacttacaaataccggttgggttctttgtttatcaggaatgctcattgtatgtagctaaaagctggccgtttatagttttattgccctaaatctggtactttatccaaaaactaaatttggaaacatcaaatacttttttcaagaatgataaactcgtacactctctagggtactcctgaaatttaaatcaaaatccaaaaccgcttaggaaggaacatatgtgataagaactgaaatttcgattaactattacaagatagtcggcccaattcgagaggactagtctccgattacaaggagtaaatatcttaatcttgataaacaaaacacatataaaaaacctaaaaatataggaacataatacataaactaaaagttgtgggaacagttacaaatctgcagtctcactccctaaatttgtgagtcacctttcacctccaagttttcgaatgttctcccaccattcactttccctccacccggattccctccaattaatagctgacacaacccgttttgacccaacattgggttcgtatcaatacatccggcccggaaaatcgacttgtcctcaagtcgaaaggaggggaattattgtgccaagcaaaaagccattcgattggaggttgatggatgatttccttgtgtttgaaagcttcaaaagatccggccaaatcagcttttaatgcctcttgaactgtagccacaacaccactttgaaacctcaaatctgttttaattaagcggccgccaccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatcgtcgaccctttccgacgctcaccgggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgtttaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgatatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatgaagatcggcggcaatagcttcttagcgccatcccggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtcgacgatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaattctagggccgctctagaggatcctatgttgtaattttatatggattaatgagaattattattattctgttcttcgtctgtgttttttaagctagcttcctcgaagcagcgtataactttaatttgaatttggttttggcgcgttagtgaaattgcggctgtaaacgtgtcaagttgtgagtggctgaaataagataatagatatattattattgttttaatttaattccgcgaagcgatatgttaagtgataaatgaaacgaagcgttttgatgacgtcatatgtctccgtgcctacgtcagcacggggcttagtattacccccgtgccgggatcagagacatttgaccaatagttgactagtataatagcccttggattaaatgacacgtggacgctcaggatctgtgatgctagtgaagcgcttaagctgaacgaatctgacggaagagcgttcacacttagatctagttagcgtacttagtacgcgttgtcttgggtctataaatagagtgcttctgaacagattgttcagaatttcatagcgccgataacaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggtaaatttctagtttttctccttcattttcttggttaggacccttttctctttttatttttttgagctttgatctttctttaaactgatctattttttaattgattggttatggtgtaaatattacatagctttaactgataatctgattactttatttcgtgtgtctatgatgatgatgataactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggtcgacggtattgagtttatgtctattgtaattgatagaggttctattaagatagaattatgagatgtaattgtgattaatgaataaagagttgttattattctttgaattactccgcgaagcggtgtgttatgtttttgttggaagctttctagactcgactaga

SEQ ID NO: 53 - DNA, 495 bp, Petunia x hybrida
catcaaactaaattaatactctggaatattagcatgcgtgaatgtcgtcgtttgtcaattgaacttcactaatagtgttaaacaatagatacagtgataaagaatagatacagtgatacaaaatcaggtcatcctgctaatcacactacactaatagtgtaaataattaaactaaagatttaaatagaagagggggcatattagtcaagttgaaagcaagtaacacgctttgcaatttttggtgaaattctggtctgcagtaaagcagcgtacaaggccacgtctggtttatatctggaaaaaggccatttcaatgtcattttcttggccagctctttctttcctttaattttaagtgatagttaaagctagggtaggtatttataaggaaagaaagacattttaagtattgagtttttcctcattcattagtgttgtaattccaagactacaaagtctttggaagtacgtgtgaggtttctgaaaggctttgaa

SEQ ID NO: 54 - DNA, 6254 bp, Nicotiana tabacum
gtttgacagcttatcatcgattatattagggatttttacacaaatagccggctatattaattgtttactttttctaaccatatacatagattatacattgattatacatgattatacacatttaatatataaattatgcatataatatacattcgctggttatttttagtttaagttatagggtgggaggctatttggattaattctttattatattaaatatttacgaatttcgtgtttgacggtgtaaatcatatgtattgatacttgttgcttcttttattaattttagtgatttagattccctagaaactaccatatgctgtttttaggttcgtataatatgagaaaagtttatttttagtcgcttccaaatatatatttatatactttctccggtccacaataagtgattttttggttgttttcacacagattaagaaattcacattttaacattaaatagcaatgaaattgatcatattaacctttactatttcttcacataaacatttctaacacatactccaacaccatttactccaagggcactgtagtaaaaaaataattaaatcatttttgaaatctaaaaaactcacttattttggaccataaaaaaagggccaaaaaataacttattgtggaccggagagagtaatacactttttggttagcgaatgcaattaatttagacattgtgttatgttccagttaaccgcttccctgcacttctttcaatctatctctcgatagaaaattgtgatactttgcgacttctatcagaggactttttgttttccatgtaacaatctgtcattttcgatggggagatttgcacaaataggctatttatgtgtcccaatttaaattttaaccccatgtcgatcagaacttagccacgagcaccagaagtttgatggatatgtgactttgtcactatccggtttactaatcaagagctatttttattcaaaattggatatctagctaagtataactggataatttgcattaacagattgaatatagtgccaaacaagaagggacaattgacttgtcactttatgaaagatgattcaaacatgattttttatgtactaatatatacatcctactcgaattaaagcgacataggctcgaagtatgcacatttagcaatgtaaattaaatcagtttttgaatcaagctaaaagcagacttgcataaggtgggtggctggactagaataaacatcttctctagcacagcttcataatgtaatttccataactgaaatcagggtgagacaaaattttggtactttttcctcacactaagtccatgtttgcaacaaattaatacatgaaaccttaatgttaccctcagattagcctgctactccccattttcctcgaaatgctccaacaaaagttagttttgcaagttgttgtgtatgtcttgtgctctatatatgcccttgtggtgcaagtgtaacagtacaacatcatcactcaaatcaaagtttttacttaaagaaattagctaaaatggtagctccaaaatgggttttcatttcttttatgattttgctaagcttagcaatatgctctggccagcctgttacctctgatgcaattaaggctaaggaagctgatcatgacaacctcaaagctcacactctgagtaatatcgacgccaaaggctttggaggaggcggtggatttggcattggtggtggttgggccggtggtggtggaggtggtggagatggtggtggttctgacacccctaactacggttataaccctggctgcagtatccatggttgcactgtccctggctttggtttcctacctaaacctgtctttggtgtcccagtctattcccctggttgtggctatgtgtgtccggccgatattcctactggaggaatgactgaatccaaaatcacaggaatatcacaatccgctagactataccgttgcaagccagggccaaatatgtgtgacagtaaagactgtaatgagcttctcctacactttgttttcccaatgcaagacaaacatgacaataaacaagaacatctaagatatggaggacgccgaggtataggtctcactgtgggaggagttggcggttttggaattggttttggtgcttggggtggtggtggtggcggaggaggtggtggttctgatgcccctggttgtagtaacgatggctgtgaccctggttttggctgtcccccgggctgtggttatgcatgtcctgccaacaatcctagtggaggaataactgaattccatatctcaggattatcacgattcgatggaccttacagatgtaggccagatatgtgtgaaagtgaagattgtaatgaacttcttctacactttgtttctccaatgcaacacaaacatgagaaccgacatgatcatatagtagaaaggagtgatgaggaggaagcgcatcatcagtcaaagcagcataaagacgaagacatcataaactaggctctcccacaaaccaaaaaaaaaggaactatatatgtagcttcagccaaaaaactgtatacactgtctaagaatactcacttccaacgaacttaaataaaactagtttacagtggattgggatataatcagttggacaatttgctaaacctcctcatgcactgtaaaaatagacttgctactagtatttggaatataatgctgaatatatttgttgttactttgcctaatgtcaatcagcattcagcaatttctctgtagttagaaaatgaaaggaagaatcaggaaactcatatttaaaggatgaaataatttaaagatcgcgaagcagtcacaatttaatagtaccaggaaaataatctataggaatcacagaactttttgatttatcaaattaaggaagcaaactgggaaaatgtgaaatgaatgaacataatgctgaaagctattgatcagatgattggattgatttcgtaggagcaacatatgatttaagattatttcaacaagatggccataaagtagcatatcatttgtaatttaacattattacactcaaactcaggaagattgtcaatttaccctcaaaacaaagttttaagccttcagtctccttcaaccacagtggcacctgcccaattggcagcacttccccggcgtggaatgctgtggagtttggtgtacaaatccacctggaaaatcacagcattgatgtttccttcatcatcttcgcgtgcaattgcttttactttgtgcagtggatgatcaaagcttattagtccagtgtcacttatgaagatacaccctgttcaatcagcttttatacattaaattttgcttgatatccaaaaaagattgaatcaaacttgaggatttgattaagaaacctggaggaaatggggtatagaattttccacaagaagcttgaatgaactctgaatcatcggaacatactaaagacccatctgcagcagttccccagtgtaacggcacacatccatcgcgatcctacattgcttagagcaacattaagtttatcctaaaaaggatttaagttatatacattgacaatataaagatttttttagtgtagtttaacctagtttaacatgtgatagaaggttagttaccacttttacccggtttccaactaatgtttatcatgtaattaccttataagtgacataactggtaaagttgctaccatgtgatcaggacatcacgggttcgaaccatgaaaacaacctcttacagaaatgcatggtaagactgcgtataatagactcttgatgtccagctcttcccggaccccgcgcatagcgggagcttagtgcaccgggctgcccttaatccttataaatgacatgattgtataaacattttttatgtcgtcagggcaccaaacttaaactcttctaaaaatactatgttgatcatggtgtatagttaagtttaaagaaacagaccaagttatggatcatggaaatggttaatatgaatgttcaataagctaagaaagagatcaattatgtgagaaaaagcattgtgactcaccctggcaagaaaaagagtagaagcttttgagtcaaacagaatgaaagcaaatttgccttcaagttctttgatgacttgatcaggagggtatggcgcccgatcccttaaaactttgtaagcttcaaccataatcatagcctctgtggcttgtcttgaaagaccataatgttttctcaaatcaaaagtgttatctaaagccccagagaatatgcagaagacatcatccatcacaacaatagacctaatgtaaacaaaacaaaacaaaacaagctcagacaacttcatttatagtcaccttgctattgcttcaatcttgaaatactacactagtttttaagaaaagttcacccttttagtatactaatcaatgtgcccaaaaagaaaaaaacaaaacatgttactattactagtactaaacatcaataccttggttgtaaagggttttcatttccatgagaaaaagccatgaaattcccattgaaaaggtggtaaaaagttgagtcttgcttccatgttttgaaactttctgcaatctcttcccttgttttagcctcctttttctgtatacctgcttgaggaaggctcaactctggaggtggcctaccaatggattgctcaaacacagccaacatgtttggacaaagtaaaagctagaaaaatttcaagaatcccaagctaatttcttttttcaagaaccataaaaatcccaggaaagcacattctatgtacttataatatagaagacttgcagctgacatgcttatccaaaagactcttgtaactgaaaacaaagtactgacgtggcatcaacaaagtgatagtgcatctgtcacactctcctttgttcaagatggtactactttaatattttatgggcgaataattactttgggaatttgtaaacaaaatttctttatttctttcattgatgggatagatgtcttggaacatggttttgaagtaccagaaaatatgatatactagtagttttagaaaggtgcatacatgcagtgggttaacaattgaatgtataacttttaatgcggagtaaaagtttattaaaattataaaaataatgaatataaactcataactttaggttcaacgctaaaaaaatttaaaagttgaacccataaaatttaaatcttgaatttgtgtctggttttaaattatgaataggacagtagaaacaagccaagaaagttttgtaaggctgaagctagcagtatatgactttacgcacttcgagaagcaggcaatactggactcttctaaataattgatccacttaaacccaaaattatttgcctatgcaacgaagtctcagaaagcacgtaataatgctatttcaagctttctgttctttcaatgatgaaactaattaagctttttttcagtggaggtcgacgtcaaggtatctttcatgtatgcattattttgatgtaatttgactaataggaagagataataagataatgtaattcccgattgagacattatttgaaaacagcaaaatctgtgggacataaccattagcggaaatgagaaaagaatggttttttgtttgaaagttacccccgcaaacgagattagcttgtgacccgtttggccatagattttgtcaaaataaatttggattttatttggcaaacatatgtttggccatagattttacctacatttcggtaaaatcaagagctggttttgagccaaaatatcactattacattttttaaaaattgcccaaacttttgtattttataaaagagcccactatttattattttgtaacaatgttacttcgtcttctcgatcacgtgatagtgtatcatgcagttcattataaaaatgataattttgtatcaaatttattcatgttcaatactatggtttgcgataatataatgaatgttattgataatggtaatgttgggtatttgtaatagtttttaaaacttgtgggtataaatcatatttcatattttttcaaaataaatttgaaaaatatgttttgaaaactgatgtccaaacacattttcatcttcaaaccaaacttcgcccaaatcagatttttcagaacaaatttgagaatctatggccaaacgctagctcagtcttctaatgcaatttgtatcaaagattccttcttcgttatcaatggaaaagcatttgtttttcttatttatacattttaattttcacagtgaa

SEQ ID NO: 55 - DNA, 346 bp, Artificial
  Synthetic construct Ca MV 35s promoter
tgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaaca

SEQ ID NO: 56 - DNA, 633 bp, Gossypium hirsutum
acaacttttctctaccaatcaaatttaaaaaatagaaaaatgaaaatcgatgaattggatcaccacaatttagcccaaagaaaaacacagtcaacccctctcacagggtaggaatgatttcgaggtatagatagacatagtaacgggcaactttaactattgctgcctcgatttgaggaaaatatcaaatccaagacaaaaatttcaattatacactatgccataccattataaatatccccgttcgcaatatcatcaccattatttgaatttgcattgcaacattcgtcaccgttagttataccatcaccatcacttaattactaaaataattattggtttctcaatatgaaaaagctcgagtgcattttcttttgaatatcaaccgaaaagaaaggaaaaactaaagattttggaaggtgacggggaaaccaaaaaggaaattttgggcatttttaaaatgagaaagacgaatgtaataacccatttttctttcttactctgacaacgccacagatgctttacatgcatcatgtgatcgtgggggacccgaaacttggcatacggaaagcaccaacggcacagcattaaaagaaattgtgtataatgttaaaagaccattaattcagtctc

SEQ ID NO: 57 - DNA, 1005 bp, Gossypium hirsutum
acaacttttctctaccaatcaaatttaaaaaatagaaaaatgaaaatcgatgaattggatcaccacaatttagcccaaagaaaaacacagtcaacccctctcacagggtaggaatgatttcgaggtatagatagacatagtaacgggcaactttaactattgctgcctcgatttgaggaaaatatcaaatccaagacaaaaatttcaattatacactatgccataccattataaatatccccgttcgcaatatcatcaccattatttgaatttgcattgcaacattcgtcaccgttagttataccatcaccatcacttaattactaaaataattattggtttctcaatatgaaaaagctcgagtgcattttcttttgaatatcaaccgaaaagaaaggaaaaactaaagattttggaaggtgacggggaaaccaaaaaggaaattttgggcatttttaaaatgagaaagacgaatgtaataacccatttttctttcttactctgacaacgccacagatgctttacatgcatcatgtgatcgtgggggacccgaaacttggcatacggaaagcaccaacggcacagcattaaaagaaattgtgtataatgttaaaagaccattaattcagtctcatccaaccacgcttaaaagtcttcatgccttttctcacctctgatttcatctaatgaaaagcggacaagttgaaggatcactcgttgcttgtgtgagctttcattatttattattatgttttaggtaaccataggaagaagccattaacaacagcatgaaaaacagctagtttctccgcaaacaagataaacttttatactttttaccactgcacccccccccaaagaccagtttttaactccacctaccaagcattcaagaagcaccaaccaacttaattaccagcttaacaagacagtacaggtttctgggatatttgtagtctctcaaggacatcaccacctccactcaccttcccatttttctctagc

SEQ ID NO: 58 - DNA, 793 bp, Arabidopsis thaliana
gaagtgccattccgcctgacctctgcacctgcgcaaggagatggtcatttcttctggttcaagcatgacatgaacaggcaataaataagttgagattttgatcacagtaactgatacttgaatcgaatcatttagatttttttttagtttacttgtttagtaaatatgttgtctatgtttgtcacaaaaacgtggctcagttcttgtatatatggagacaaaaaaatccattaaaagattgttgacattctcggaaatttagtgccaactgttattgcgagaacttactatagttttcctttggcgaaaagctaataatcttaatcttgattttgtcctcttttctctgagttagattttcttaaattccacttccgacctattaagaaatgggcttttgcaaagaagatccgcttcactgagcccgtatctcgaagaggataatacaacaacaaagcaaaacggcacgtagttttaattgtaaccaaggattgcatttcggtcttgtttcaacaaacgaaacttcctgaaatgccaagaaaaatctggtcatttcaccacagtgatcattgtgtatgtgttctaaagactccaagcgaaggttttagaaaaaggagcattttctattctattcaagaaactcgaagaacattctctcttcatcctctaacttccctataaatatgtcctttgctaatcagatcaaatcagcaggaaaatcaagaaccaaaagtctcccgaaaagcaacgaacaaatgttgcgcaggtgcagaggctaggtggaggctcagtg

SEQ ID NO: 59 - DNA, 584 bp, Glycine max
gaagtgccattccgcctgacctctgcacctgcgcaaggagctagatagtcagccttttaagagatagaatttaaaatataatttgcgtaaaacattattaaaaatacaaatttataaattaagttcaactcatcctatctcactctttaaatacgatgtttacttattagactcattaataaaaaaaaatcatttgtacaaagcccaccataaaggcaatttgggcctggtagaccaatcctaaccaatgtctggttaagatggtccaatcccgaaacttctagttgcggttcgaagaagtccagaatgtttctgaaagtttcagaaaattctagttttgagattttcagaagtacggcatgatgatgcataacaaggactttctcgaaagtactatattgctcctctacatcattttaaataccccatgtgtcctttgaagacacatcacagaaagaagtgaaggcatcgttagcagttttgtagattcaacctcaatttgcagagttacgttctaatatatttacacaagactgataagagaaaatgttgcgcaggtgcagaggctaggtggaggctcagtg

SEQ ID NO: 60 - DNA, 1746 bp, Arabidopsis thaliana
ctgcacctgcgcaaggagatcattgtgcacaatttaacagaatgtaatttcttgtaaaagaagaggaaacaattcgctgttagtaaatgtgaagtatagaagtctaaaatgagatacctcaactagcttgaattaagaaaaaaaacaaaaactctatcgacatgaaaaaggtcgcaaatatttatcatttatcaatgccaaaggagtatttggttcacaaaatactgaatcatttatatagatatataattagctctaaattctactataacttgcaaaataagtatactgactcaattatatagcgtttaaaaatagacgatttgtatgatgaggtccatatatatggagatgtgcatgcaactatcgacattttcacacgttgatatcgtctttctccaatggagacttgaatttgtgtaaactatgaatactcgtctctctaagaccttttttcttcaaccatgccaactatttaggtaagattttactgtctttgattgatattaaatacttagccgtggcgttatcaatgaatgataataaaaatgcggataaaagccaaaggtgttggaaataaatccaagaatgaagacgtagatgtcgatgggtattttaagaacttgaatttgtcacgactcacacgttaaaatatattatccgaattgtttagtctaaagacacacatatattgaaaaagaaaaggtaaatgaagctcattggtgcctaaatgtgaaatgaagccgaaatgtgttaggtgaacacatttaaatatacaaaaagaaatataatagaaacaaaactaattaacaaagtcgcaatttgtattgtataaaatatctttccgtctcccgtcatatttgaaaaaaaaaaaattacaaatctgttaattttaaaactttctagaaaaacacaagtatataattttctcttttcgtgcgtgtttgttttaaaataacattgttttgattggcgactcaacatattttagcatttacatatttctgcatatattaaatgatttataaactcaactatagattaaaatataatttgacatctaataattttaacaataatataaaatatgagatttataaattacgaatataaatattcaagggagagaaaaagtagaacataattcaaaagataagactttttagacttttttaacaatatttttgatggataaaaattattcaaaagagaagaaagtaagaagaaaagatgtttctgagaatttctagaaacagcatccgtttttataatttaattttcttacaaaggtaggaccaacatttgtgatctataaatcttcctactacgttatatagagacccttcgacataacacttaactcgtttatatatttgttttacttgttttgcacatacacacaaaaataaaaaagactttatatttatttactttttaatcacacggattagctccggcgaagtatggtcgtcgtcttcatcttcttcctccatcatcagatttttccttaaatggaagaaaccaaacgaaactccgatcttctccgttctcgtgttttcctctctggcttttattgctgggattgggaatttctcaccgctctcttgctttttagttgctgattctttttccttcgactttctatttccaatctttcttcttctctttgtgtattagattatttttagttttatttttctgtggtaaaataaaaaaagttcgccggagaatgttgcgcaggtgcag

SEQ ID NO: 61 - DNA, 682 bp, Gossypium hirsutum
gaagtgccattccgcctgacctctgcacctgcgcaaggaggcaacaaaaaatgggcatcgtctccctcagaatcaccattcgcccattcaagaaaaagccccaaagttaccatttattattttaaatattaaaaatttatttattatttccataaaaaatatttaaaaaacaatttgattctttaatttaaacttcttggaagcatggtaaaagtcagtccaccaagcaccaaccccgcaacttgcagagaaaattaaatccatgtgggccgaatgacaaagacaagtgggcccactttactttaacgagtaactttccgtcaatgacgacgtctccttcccttttctttctttcactttaaaccaaacacctttaaacggaataactaacggcagtgagacccaccacctaaccttatattattagattcctattccctatgcccaattgattaagtaaccgttacgtgtccaataacatttaaacgtcacgtcttcttacgtgtcgaatttttattcgtttgtcattttcgaagttttcctataaaggaagcccaccgcttgccttattttctatctgaagcaatttgtttgctctgtatattgaaattttctttctcgattttctctttagccaaaaaagaatgttgcgcaggtgcagaggctaggtggaggctcagtg

SEQ ID NO: 62 - DNA, 2250 bp, Artificial
  Synthetic construct dexmethazone inducible promoter and synthetic transcription factor fusion
ctgcacctgcgcaaggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgatggcagatccaatgaagctactatcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtccccgctgactagggcacatctgacagaagtggaatcaaggctagaaagactggaacagctatttctactgatttttcctaggtcgagcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggatccaattcagcaagccactgcaggagtctcacaagacacttcggaaaatcctaacaaaacaatagttcctgctgcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgataccaggcttcagaaacttacacctggatgaccaaatgaccctgctacagtactcatggatgtttctcatggcatttgccctgggttggagatcatacagacaatcaagtggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaatgaggactcccatgttggcaaaggcaaccaaacaaacaatgaatgatccgctcctgcatatggggcggtttgagtatttcaactgccatttgggctgaattgtagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatccttgcatgccggtcgactctagaggatccgggtgacagccctccgacgggtgacagccctccgacgggtgacagccctccgaattctagaggatccgggtgacagccctccgacgggtgacagccctccgacgggtgacagccctccgaattcgagctcggtacccggggatctgtcgacctcgatcgagatcttcgcaagacccttcctctatataaggaagttcatttcatttggagaggacacgctgaagctagtcgactctagaatgttgcgcaggtgcag

SEQ ID NO: 63 - DNA, 3280 bp, Artificial
  Synthetic construct dexamethazone inducible promoter and sythetic transcription fusion
ctgcacctgcgcaaggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgatgatttctggaattcggtctctaatggctagtgaagctcgaaaaacaaagaaaaaaatcaaagggattcagcaagccactgcaggagtctcacaagacacttcggaaaatcctaacaaaacaatagttcctgcagcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgataccaggcttcagaaacttacacctggatgaccaaatgaccctgctacagtactcatggatgtttctcatggcatttgccctgggttggagatcatacagacaatcaagtggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaatctactagcaaaccggtaacgttatacgacgtcgctgaatacgccggcgtttctcatcaaaccgtttctagagtggttaaccaggcttcacatgttagcgctaaaacccgggaaaaagttgaagctgccatggctgagctcaactacatcccgaaccgtgttgcgcagcagctggctggtaaacaaagcttgctgatcggtgtcgcgacctcgagcttggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaggacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaggacagctcatgttatatcccgccgttaaccaccatcaaacaggattttcgcctgctggggcaaaccagcgtggaccgcttgctgcaactctctcagggccaggcggtgaagggcaatcagctgttgcccgtgtcactggtgaaaagaaaaaccactagtggatcggaattcgccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagtaaggactcccatgttggcaaaggcaaccaaacaaacaatgaatgatccgctcctgcatatggggcggtttgagtatttcaactgccatttgggctgaattgtagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatccttgcataattgtgagcgctcacaattgaaagactagaaagaagaaagggaagagaaagaattgtgagcgctcacaattgaaagactagaaagaagaaagggaagagaaagaattgtgagcgctcacaattgaaagactagaaagaagaaagggaagagaaagaattgtgagcgctcacaattgaaagactagaaagaagaaagggaagagaaagaattgtgagcgctcacaattgaaagactagaaagaagaaagggaagagaaagaattgtgagcgctcacaattgaaagactagtggatcgatcttcgcaagacccttcctctatataaggaagttcatttcatttggagaggacacgaatgttgcgcaggtgcag

SEQ ID NO: 64 - DNA, 3993 bp, Artificial
  Synthetic construct genes sufficient to enact betailin synthesis, fused with cleavable peptide linker P2a
ctgcacctgcgcaaaatgatggatcatgcgaccctcgccatgatcctcgcgatctggttcatcagcttccacttcatcaagctgctgttctcccagcagaccaccaagctgcttccgccaggaccaaagccgcttccgatcatcggcaacatccttgaggtgggcaagaagccgcatcggtccttcgccaacctcgccaagattcacggcccactcatttccctcagactcggctctgtgaccaccatcgttgtgtcctctgccgacgtggccaaagagatgttcctcaagaaggatcacccgctctccaaccgcacgatcccgaatagtgttacagccggcgaccaccacaagctcaccatgtcttggctcccggtgtctccgaagtggcgcaacttccgcaagattaccgccgtgcatctgctctccccacagagactcgatgcctgccagacattcaggcacgccaaggtgcagcagctctacgagtacgttcaagagtgcgcccagaaaggccaggccgtggatattggcaaggccgcctttacgaccagcctcaacctcctcagcaagctgttcttcagcgtcgagctggcgcaccacaagtcccataccagccaagagttcaaagagctgatctggaacatcatggaagatataggcaagccgaactacgccgactacttcccgattctcggctgcgttgacccatctggcattagaagaaggctcgcctgctccttcgacaagctgatcgccgtgttccagggcatcatctgcgagagactcgccccagattcctccaccacaactaccaccaccaccgacgacgtgctcgatgtgctcctccagctgttcaagcagaacgagctgacgatgggcgagatcaaccacctcctcgtggacatcttcgacgccggcaccgataccacatcctccacattcgagtgggtgatgaccgagctgatccgcaatccagagatgatggaaaaggcccaagaggaaatcaagcaggtcctcggcaaggacaagcagatccaagagtccgacatcatcaacctgccgtacctccaggcgatcatcaaagagacactccgcctccatccgccgaccgtgttcttgctcccaagaaaggccgacaccgatgtcgagctgtacggctacatcgtgccgaaggatgcccagatcctcgtgaacctctgggccattggcagggacccaaacgcctggcagaacgccgatattttcagcccagagcgcttcatcggctgcgagatcgatgttaagggccgcgatttcggcctccttccatttggcgctggccgcagaatttgcccaggcatgaatctcgccatcaggatgctcaccctcatgctcgccacactcctccagttcttcaactggaagctcgaaggcgacatctccccgaaggacctcgacatggacgagaagttcggcattgcgctccaaaagaccaagccgctcaagctcatcccgattccgcgctacggaagcggagctactaacttcagcctgctgaagcaggctggagacgtggaggagaaccctggacctatgaagatgatgaacggcgaggacgccaacgaccagatgatcaaagagtccttcttcatcacccacggcaacccgatcctcaccgtcgaggatacacatccgctcaggccgttcttcgagacatggcgcgagaagattttctccaagaagccgaaggccatcctcatcatctccggccactgggagacagtgaagccaaccgtgaacgccgtgcacatcaacgacaccatccacgacttcgacgactacccagccgccatgtaccagttcaagtacccagctccaggcgagccagagcttgcgagaaaggtggaagagatcctcaagaagtccgggttcgagacagccgagacagaccaaaagaggggccttgatcacggcgcctgggttccactcatgctcatgtatccagaggcggacatcccggtgtgccagctctcagttcagccacatctcgacggcacctaccactacaatctcggcagagccctcgcgccgctcaagaatgatggcgtgctcattattggctccggcagcgccacacatccactcgatgagacaccgcactacttcgatggtgttgccccttgggccgctgccttcgattcttggcttaggaaggccctcatcaacggccgcttcgaggaagtgaacatctacgagagcaaggccccgaactggaagctcgcccatccatttccagagcacttctacccgctccacgttgtgctcggcgctgctggtgaaaagtggaaggccgagctgatccactcctcctgggatcatggcacactttgccacggctcctacaagttcacctccgccggttcaggtgccactaatttttctctcttgaaacaggccggtgacgttgaagagaacccaggtccaatgaccgccatcaagatgaacaccaacggcgagggcgagacacagcacatcctcatgatcccgttcatggcgcagggccacctcaggccatttctcgaactcgccatgttcctctacaagcgctcccacgtgatcatcaccctgctcacaactccgctcaacgccggcttcctcaggcacctccttcaccaccattcctactcctccagcggcatcaggatcgtcgagctgccattcaactccaccaaccacggactcccaccgggcatcgagaacaccgataagctcacactcccgctcgtggtgtccctcttccattccaccatcagcctcgatccgcacctccgcgattacatctccaggcatttcagcccagccaggccaccactctgcgtgatccatgatgtgttcctcggctgggttgaccaggtggccaaggatgtgggctctacaggcgtggtgttcacaacaggcggcgcttatggcacatccgcctacgtgtccatctggaacgatctcccgcaccagaactactccgacgaccaagagttcccgctgccaggcttcccagagaaccataagttccgcaggtcccagctccatcggttcctcagatatgccgacggctccgacgattggtccaagtatttccagccgcagctccgccagtccatgaagtcttttggctggctctgcaactccgtggaagagatcgagacactcggcttctccatcctccgcaactacaccaagctgccgatctggggcatcggcccacttattgcttccccagtgcagcactcctcctccgacaacaattcaacaggcgccgagttcgtgcagtggctcagcctcaaagagccggactccgtcctctacatctccttcggctcccagaacacgatcagcccgacgcagatgatggaactcgctgctggccttgagtcctccgagaagccattcctctgggtgatcagagccccgttcggcttcgacatcaacgaagagatgcgcccagagtggctgccagagggctttgaggaacgcatgaaggtgaagaaacagggcaagctcgtgtacaagctcggcccgcagcttgagatcctcaaccatgaatccatcggcggctttctcacccactgcggatggaacagcatccttgagtctcttcgcgagggcgttccgatgcttggatggccacttgctgccgagcaggcctacaacctcaagtacctcgaagatgagatgggcgtcgcggttgagcttgctagaggcctcgaaggcgagatctccaaagagaaggtcaagcgcatcgtcgagatgatccttgagcgcaacgagggctccaaaggctgggagatgaagaatcgcgccgtggaaatgggcaaaaagctcaaggacgccgtgaacgaggaaaaagagctgaagggctcctccgtgaaggcgatcgacgatttcctcgacgccgtcatgcaggccaaacttgagccaagcctccagtgatagtgagcttttgcgcaggtgcag

SEQ ID NO: 65 - DNA, 1543 bp, Artificial
  Synthetic construct WUS from Arabidopsis under the constituive mas promoter/terminator from agrobacterium, to enforce stem cell identity and promote sensitiviity to other treatments
cacctgcgcaaaatgatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgatggagccgccacagcatcagcatcatcatcatcaagccgaccaagaaagcggcaacaacaacaacaacaagtccggctctggtggttacacgtgtcgccagaccagcacgaggtggacaccgacgacggagcaaatcaaaatcctcaaagaactttactacaacaatgcaatccggtcaccaacagccgatcagatccagaagatcactgcaaggctgagacagttcggaaagattgagggcaagaacgtcttttactggttccagaaccataaggctcgtgagcgtcagaagaagagattcaacggaacaaacatgaccacaccatcttcatcacccaactcggttatgatggcggctaacgatcattatcatcctctacttcaccatcatcacggtgttcccatgcagagacctgctaattccgtcaacgttaaacttaaccaagaccatcatctctatcatcataacaagccatatcccagcttcaataacgggaatttaaatcatgcaagctcaggtactgaatgtggtgttgttaatgcttctaatggctacatgagtagccatgtctatggatctatggaacaagactgttctatgaattacaacaacgtaggtggaggatgggcaaacatggatcatcattactcatctgcaccttacaacttcttcgatagagcaaagcctctgtttggtctagaaggtcatcaagaagaagaagaatgtggtggcgatgcttatctggaacatcgacgtacgcttcctctcttccctatgcacggtgaagatcacatcaacggtggtagtggtgccatctggaagtatggccaatcggaagttcgcccttgcgcttctcttgagctacgtctgaactagtggactcccatgttggcaaaggcaaccaaacaaacaatgaatgatccgctcctgcatatggggcggtttgagtatttcaactgccatttgggctgaattgtagacatgctcctgtcagaaattccgtgatcttactcaatattcagtaatctcggccaatatcctaaatgtgcgtggctttatctgtctttgtattgtttcatcaattcatgtaacgtttgcttttcttatgaattttcaaataaattatcttcgttgcgcaggtg

SEQ ID NO: 66 - DNA, 1562 bp, Artificial
  Synthetic construct trichome initiation gene MYB2 from gossypium arborium fused to the heat inducible promoter from Arabidopsis
cacctgcgcaaggagatggtcatttcttctggttcaagcatgacatgaacaggcaataaataagttgagattttgatcacagtaactgatacttgaatcgaatcatttagatttttttatttttttagtttacttgtttagtaaatatgttgtctatgtttgtcacaaaaacgtggctcagttcttgtatatatggagacaaaaaaatccattaaaagattgttgacattctcggaaatttagtgccaactgttattgcgagaacttactatagttttcctttggcgaaaagctaataatcttaaatcttgattttgtcctcttttctctgagttagattttcttaaattccacttccgacctattaagaaatgggcttttgcaaagaagatccgcttcactgagcccgtatctcgaagaggataatacaacaacaaagcaaaacggcacgtagttttaattgtaaccaaggattgcatttcggtcttgtttcaacaaacgaaacttcctgaaatgccaagaaaaatctggtcatttcaccacagtgatcattgtgtatgtgttctaaagactccaagcgaaggttttagaaaaaggagcattttctattctattcaagaaactcgaagaacattctctcttcatcctctaacttccctataaatatgtcctttgctaatcagatcaaatcagcaggaaaatcaagaaccaaaagtctcccgaaaagcaacgaacaatggctccaaagaaggatggagtgagcaaaagggtttttaacaaaggttcttggacagctgaggaagatagaagattggctaaatatattgagattcatggcgcaaagagatggaaaacaatcgccattaaatcaggtttgaatcgatgcggcaagagttgcaggttgagatggttgaactacttgagacctaacattaagagaggcaacatatcagatgaagaagaggacttaattattaggcttcataaactgctggggaacaggtggtctttgattgctgggagacttccagggcgaacagacaatgaaattaagaactactggaattcccatttgagcaagaaaataataaaccatgatgtcagaacagaacaaacttcctcctcggaacaaattgtgcctcacaaagcatgggaaactgtccagatggaagaagaagaggtagtaaaaggaagtgatgaaattgaaaactctgaattcagcattgatgtggacgaattctttgacttcacaacggaaggttgctttagtttggattgggtgaataagttccttgaacttgatgatcaacaggatccattagcaatggtataaagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcaatgttgcgcaggtg

SEQ ID NO: 67 - DNA, 1855 bp, Artificial Sequence
  Synthetic construct fiber initiation gene MYB25-like from gossypium hirsutum fused to the heat inducible promoter from Arabidopsis
cacctgcgcaattcgatggtcatttcttctggttcaagcatgacatgaacaggcaataaataagttgagattttgatcacagtaactgatacttgaatcgaatcatttagatttttttatttttttagtttacttgtttagtaaatatgttgtctatgtttgtcacaaaaacgtggctcagttcttgtatatatggagacaaaaaaatccattaaaagattgttgacattctcggaaatttagtgccaactgttattgcgagaacttactatagttttcctttggcgaaaagctaataatcttaaatcttgattttgtcctcttttctctgagttagattttcttaaattccacttccgacctattaagaaatgggcttttgcaaagaagatccgcttcactgagcccgtatctcgaagaggataatacaacaacaaagcaaaacggcacgtagttttaattgtaaccaaggattgcatttcggtcttgtttcaacaaacgaaacttcctgaaatgccaagaaaaatctggtcatttcaccacagtgatcattgtgtatgtgttctaaagactccaagcgaaggttttagaaaaaggagcattttctattctattcaagaaactcgaagaacattctctcttcatcctctaacttccctataaatatgtcctttgctaatcagatcaaatcagcaggaaaatcaagaaccaaaagtctcccgaaaagcaacgaacaatgcaacagtcaccatgctctgacaaggtcgggctaaagaaaggaccgtggacacccgaagaagaccagaaattgctgtcgtatatacaggagcacggtggtggttcgtggagaggtcttccggcaaaagccggtctgcaacgttgtggaaagtcctgccgacttagatggatcaactacttacgtcctgacataaagagaggaaaatttagttcccaggaagaacgtactataatccagctgcatgccttgttgggtaacaggtggagcgcgatcgctgcccaccttccgaagagaaccgacaatgaaataaagaactattggaacacccaattaaagaagcgtttaactaccattgggatcgatccggccacacaccggcctaagactgatacccttggatctacacccaaggacgcggcgaacctaagtcacatggcgcagtgggaatctgcacggttggaagcggaagcaaggctggttagagagtctaagagggttagcaatccacctcagaatcaatttcgtttcacatctagttctgccccacctcttgtttcaaagatcgatgtggggctcgcgcacgctaccaagccccaatgcttggacgttttgaaagcctggcagcgagtggtaacagggttatttactttcaatactgacaatctgcagtcacccactagtactagcagtttcacggagaatactttgccaatttccagtgtgggtttcatcgacagcttcgtcgggaattctaataatagttgttgtggcaacaattgggagtgcgtcgaaaagtcatcccaggtcgctgagcttcaagagatcttagacaattctatgggccttcatgacattctcgacctttcctcagaggacgtttggttccagggaagctacagagcagagaacatgatggaaggatattcggacacacttatggtctgtgactctggtgatcatccaaagtcattgtctatggagccgaggcaaaactttaatgtggggacgtcaaacgcgagctcattcgaagaaaataagaactactggaacaatatcctcaactttgcgaatgctagtccgagtggatcttctgtattttaggcttttgcgcaggtg

SEQ ID NO: 68 - DNA, 2134 bp, Artificial
  Synthetic construct fiber specific expression from SCFP gene driving the fiber promoter gene FP1 from gossypium hirsutum
cacctgcgcaattcgtttattacaagttttctctaccaatcaaatttaaaaaatagaaaaatgaaaatcgatgaattggatcaccacaatttagcccaaagaaaaacacagtcaacccctctcacagggtaggaatgatttcgaggtatagatagacatagtaacgggcaactttaactatagctgcctcgatttgaggaaaatatcaaatccaagacaaaaatttcaattatacactatgccataccattataaatatccccgttcgcaatatcatcaccattatttgaatttgcattgcaacattcgtcaccgttagttataccatcaccatcacttaattactaaaataattattggtttctcaatatgaaaaagctcgagtgcattttcttttgaatatcaaccaaaaagaaagaaaaaactaaagattttggaagatgacggggaaaccaaaaaggaaattttgggcatttttaaaatgagaaagacgaatgtaataacccatttttctttcttactctgacaacgccacagatgctttacatgcatcatgtgatcgtgggggacccgaaacttggcatacggaaagcaccaacggcacagcattaaaagaaattgtgtataatgttaaaagaccattaattcagtctcatccaaccacgcttaaaagtcttcatgccttttctcacctctgatttcatctaatgaaaagcggacaagttgaaggatcactcgttgcttgtgtgagctttcattatttattattatgttttaggtaaccataggaagaagccattaacaacagcatgaaaaacagctagtttctccgcaaacaagataaacttttaaactttttaccactgcaccccccccaaagaccagtttttaactccacctaccaagcattcaagaagcaccaaccaacttaattaccagcttaacaagacagtacaggtttctgggatatttgtagtctctcaaggacatcaccacctccactcaccttcccatttttctgtagcaccccccaaaaagcttgtgcttggatatggagtcaaacgcggggacattggagaccagatctaatagaacggcgttggaaagggaggaagtagcggcacttgaatcgctacaatttaccgacgaaatccaccgtttgatgtcggctccggcgaccgacaatgccagttccttcacggctttgcttgaactccctgctccccaagctgttcagcttattcactctccggattcggctaagctcattgccactccagctccgaacgttgaagatttcaaaggcgggtttcatttcccttctaatggtgggctaattgaacgagcagccaggttctccgtgttcgccggcgaaggtaaaaacaagagcgactcgcctgaagcgacgtcgaacaattcaagtgcaaatctccagaaggcagtgaagagtgaaccggctgagaccgagtcgtctcagccgttaatttccgatccaacggtggagaaccggggtatcaagagaaaagaccgagaaaaggtgaaagggcagacgaagaaaagcaaaacaacggcgaaagagagctccgacgacgccgagaagctgccttacgttcacgttagagctcgccggggacaagcaacggatagccatagcttagctgagagagcaaggagagagaagattaatgcacgtatgaagctcctacaggagttagtaccaggatgcaataagatttcgggcacagcgttagttttggatgaaatcatcaaccatgtgcagtccctacaacgtcaagtggagttcttatcaatgacactcgctgcagtgaacccaagaattgatttcaactttgatagtgtattcacggcagaaagtggatctttaatggacagtaacttttctggcatggttatgcctgtgatgtggcccgaggttcaactgaacagcaacagacaacagtatccgcagcaattgcattttgatgcatttcagcaacctgtttgggggagagaagaagtctgcaataattacttaactccggagaactcgcttttaagctatgattcctctgcaaactcagtgtcgttgcactcgaatcaattgaaaatggagctatgagcttttgcgcaggtg

SEQ ID NO: 69 - DNA, 2225 bp, Artificial
  Synthetic construct nptII gene translationaly fused to DSRED2 reporter with P2A self cleaving linker
ctgcacctgcgcaatgccgggctattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacaaatcaccagtctctctctacaaatctatctctatggcgtctagcgaaaacgttataaccgagttcatgagatttaaagtgaggatggaaggaactgtgaatggtcatgagtttgaaatcgaaggagaaggagagggtcgtccgtatgaaggtcataataccgttaaattaaaagttacaaaaggcggtcctcttccatttgcttgggacatcctctctccacagttccagtatggatcgaaagtatatgtcaaacaccccgccgacatccctgattacaaaaagttgtcttttcctgagggttttaaatgggaacgtgttatgaattttgaggatggaggtgtcgccacggttactcaggatagttcacttcaggatggttgtttcatatataaagtcaaatttatcggagtgaattttccatcagatggtccagtaatgcaaaagaaaactatgggatgggaagcttccacagagcgactttaccccagagatggtgttctaaagggggaaactcataaagctctcaagctcaaggatggcggacattacttggtggagttcaagagcatatacatggcgaaaaaacctgtccaactaccgggctattactacgtggacgcaaagcttgacataacgtctcataatgaggattataccattgttgaacaatacgaaagaactgagggtagacatcacctttttctaggaagcggagctactaacttcagcctgctgaagcaggctggagacgtggaggagaaccctggacctatggtggagcaggacgggctacatgctggaagtcctgctgcgtgggtcgaaagattgtttggatacgattgggctcagcaaactatagggtgtagcgacgctgcagtctttagactttctgctcaaggtcgtcctgtgctgttcgtgaaaacagatttgtctggagctttgaacgagctccaagatgaagctgctcgactctcatggctcgctaccactggtgtgccatgtgccgccgttcttgatgtggtgacagaggctggccgtgattggcttctgttaggagaggttccaggtcaagaccttctttctagccatttagccccagcagaaaaggtttcaataatggccgacgccatgagaagactccacacactagaccccgcaacatgtccttttgatcatcaggctaagcaccgtattgaaagggctagaaccaggatggaagctggattggtggaccaagacgacctcgatgaggaacaccaaggacttgctccggcagagctctttgctcgtcttaaagcccgtatgcctgatggagaagacctagttgttacacacggagacgcttgtctccctaacattatggtcgagaacggtaggttctccggttttattgattgcggacgactaggagtcgctgataggtaccaagatattgctcttgcgacgagagacatcgcagaggaactaggtggtgagtgggcagatagatttctagttctatatggaattgcggcacctgactctcaaaggattgcattttaccgattgttggacgaattcttctgagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaagggcggagttgcgcaggtgcag